(19) United States
(12) Patent Application Publication
(10) Pub. No.: US 2014/0107151 A1
(43) Pub. Date: Apr. 17, 2014

(54) TYROSINE KINASE INHIBITORS

(71) Applicant: Principal Biopharma Inc., South San Francisco, CA (US)

(72) Inventors: David Michael Goldstein, Redwood City, CA (US); Kenneth Albert Brameld, Menlo Park, CA (US)

(73) Assignee: Principia Biophama Inc., South San Francisco, CA (US)

(21) Appl. No.: 14/117,870

(22) PCT Filed: May 16, 2012
(86) PCT No.: PCT/US2012/038163

§ 371 Date: Nov. 15, 2013

Related U.S. Application Data
(60) Provisional application No. 61/486,950, filed on May 17, 2011.
(60) Provisional application No. 61/600,311, filed on Feb. 17, 2012.

(51) Int. Cl. C07D 471/04; A61K 45/06; A61K 31/4545; C07D 401/12; A61K 31/454; A61K 31/4439; A61K 31/437; A61K 31/444

(52) U.S. Cl. CPC C07D 471/04; A61K 31/437; A61K 45/06; A61K 31/444; C07D 401/12; A61K 31/454; A61K 31/4439; A61K 31/4545
USPC 514/303; 546/119; 546/199; 514/322; 546/275.7; 514/338

(57) ABSTRACT

The present disclosure provides compounds and pharmaceutically acceptable salts that are tyrosine kinase inhibitors, in particular BLK, BMX, EGFR, HER2, HER4, ITK, JAK3, TEC, BTK, and TXK and are therefore useful for the treatment of diseases treatable by inhibition of tyrosine kinases such as cancer and inflammatory diseases such as arthritis, and the like. Also provided are pharmaceutical compositions containing such compounds and pharmaceutically acceptable salts and processes for preparing such compounds and pharmaceutically acceptable salts.

The present disclosure provides compounds and pharmaceutically acceptable salts that are tyrosine kinase inhibitors, in particular BLK, BMX, EGFR, HER2, HER4, ITK, JAK3, TEC, BTK, and TXK and are therefore useful for the treatment of diseases treatable by inhibition of tyrosine kinases such as cancer and inflammatory diseases such as arthritis, and the like. Also provided are pharmaceutical compositions containing such compounds and pharmaceutically acceptable salts and processes for preparing such compounds and pharmaceutically acceptable salts.

The human genome contains at least 500 genes encoding protein kinases. Many of these kinases have been implicated in human disease and as such represent potentially attractive therapeutic targets. For example EGFR is overexpressed in breast, head and neck cancers and the overexpression is correlated with poor survival (see Do N. Y., et al., Expression of c-erbB receptors, MMPs and VEGF in squamous cell carcinoma of the head and neck. Oncol Rep. August 12:229-37. 2004 and Foley J, et al. EGFR signaling in breast cancer: bad to the bone. Semin Cell Dev Biol. 21:951-60. 2010). Her2, another EGFR family member, also is amplified or overexpressed in up to 30% of breast cancers, also correlating with poor survival (see Murphy C. G, Modi S. HER2 breast cancer therapies: a review. Biologics 3:289-301. 2009). HER4, also in the EGFR family, is overexpressed in head and neck squamous cell carcinomas (see Rosen F. S., et al. The primary immunodeficiencies. New Engl. J. Med. 333:431-40. 1995). Other studies show decreased expression of HER4 in certain cancers and suggest tumor suppressor activity (see Thomasson M, et al., ErbB4 is downregulated in renal cell carcinoma—a quantitative RT-PCR and immunohistochemical analysis of the epidermal growth factor receptor family. Acta Oncol. 43:453-9. 2004). Overall the data support a role for members of the EGFR family in cancer. ITK, a member of the TEC kinase family, is involved in activation of T cells and mast cells (see Iyer A. S. et al. Absence of Tec Family Kinases Interleukin-2 Inducible T cell Kinase (Itk) and Bruton's Tyrosine Kinase (BTK) Severely Impairs Fc{epsilon}RI-dependent Mast Cell Responses. J. Biol. Chem. 286:9503-13. 2011) and is a potential target in inflammatory immune diseases such as asthma. Mice deficient in ITK are resistant to development of allergic asthma (see Sahu N, et al., Differential sensitivity to Itk kinase signals for T helper 2 cytokine production and chemokine-mediated migration. J. Immunol. 180:3833-8. 2008). Another family member, BMX, is involved in supporting tumor angiogenesis through it's role in the tumor vascular endothelium (see Tu T, et al., Bone marrow X kinase-mediated signal transduction in irradiated vascular endothelium. Cancer Res. 68:2861-9. 2008) and is also progressively up-regulated during bladder cancer progression (see Guo S, et al., Tyrosine Kinase ETK/BMX Is Up-Regulated in Bladder Cancer and Predicts Poor Prognosis in Patients with Cystectomy. PLoS One. 6:e17778. 2011) suggesting a potential therapeutic target in this type cancer. JAK3, which is critical for signaling downstream of IL-2 as well as other cytokines that utilize the common gamma chain of the IL-2 receptor, has clinical utility for a number of indications including rheumatoid arthritis, kidney transplantation, Crohn's disease, psoriasis, and JAK3-dependent hematopoietic malignancies (see Ghoreschi K, et al., Janus kinases in immune cell signaling. Immunol Rev. 228:273-87. 2009). The B lymphoid kinase (BLK) is linked through genetic association with a variety of rheumatic diseases including systemic lupus erythematosus and systemic sclerosis (see Ito I, et al., Association of the FAM167A-BLK region with systemic sclerosis. Arthritis Rheum. 62:890-5. 2010).

Bruton's tyrosine kinase (abbreviated as BTK), a member of the Tec family non-receptor tyrosine kinases that is essential for B cell signaling downstream from the B-cell receptor. It is expressed in B cells and other hematopoietic cells such as monocytes, macrophages and mast cells. It functions in various aspects of B cell function that maintain the B cell repertoire (see Gauld S. B. et al., B cell antigen receptor signaling: roles in cell development and disease. Science 296:1641-2. 2002). Clinical validation of the role of B cells in RA has been provided by the efficacy of Rituxan (an anti-CD20 antibody), which depletes B cells as a mechanism of action (see Perosa F., et al., CD20-depleting therapy in autoimmune diseases: from basic research to the clinic. J Intern Med. 267:260-77. 2010 and Dorner T, et al. Targeting B cells in immune-mediated inflammatory disease: a comprehensive review of mechanisms of action and identification of biomarkers. Pharmacol Ther. 125:464-75. 2010). BTK is known to be required for B cell development because patients with the disease X-linked agammaglobulinemia (see Rosen F. S., et al., The primary immunodeficiencies. N Engl J. Med. 333:431-40. 1995). Notably, small-molecule BTK inhibitors in pre-clinical development have been shown to be efficacious in collagen-induced arthritis (see Pan Z., et al., Discovery of selective irreversible inhibitors for Bruton's tyrosine kinase. J. Med. Chem. 2:58-61. 2007). However, the potential advantage of a BTK inhibitor (beyond the inherent advantage of a small-molecule over a biologic) is that modulation of BTK can inhibit B cell function without permanent removal of the B cell itself. Therefore, the long periods of low B cell levels experienced with Rituxan should be avoidable by targeting BTK.

In addition, the disease modifying activities of BTK are expected to extend beyond those of Rituxan because of effects on addition cellular targets that are involved in propagation of disease. For instance, antigen induced mast cell degranulation is impaired in mast cells derived from the bone marrow of BTK deficient mice, demonstrating that BTK is downstream of the FcεR1 receptor (see Setoguchi R., et al., Defective degranulation and calcium mobilization of bone-marrow derived mast cells from Xid and BTK-deficient mice. Immunol Lett. 64:109-18. 1998). A similar signaling module exists in monocytes and macrophages for the FcγR1 receptor indicating BTK inhibition is highly likely to modulate TNF production in response to IgG. Both mast cells and macrophages are thought to contribute to propagation of the inflammatory cytokine environment of the diseased synovium.

In addition to the peripheral and synovial effects of BTK inhibition described above, there is evidence that BTK inhibition will have bone protective effects in the inflamed joint (see Gravallese E. M., et al., Synovial tissue in rheumatoid arthritis is a source of osteoclast differentiation factor. Arthritis Rheum. 43:250-8. 2000). Studies with mice that are either deficient in BTK or have impaired BTK function have demonstrated that Rank ligand-induced osteoclast differentiation is impaired in the absence of BTK function (see Lee S. H., et. al., The tec family tyrosine kinase BTK Regulates RANKL-induced osteoclast maturation. J. Biol. Chem. 283:11526-34. 2008). Taken together these studies suggest a BTK inhibitor could inhibit or reverse the bone destruction that occurs in RA patients. Given the importance of B cells in autoimmune disease, BTK inhibitors could also have utility in other autoimmune diseases such as systemic lupus erythematosus (see Shlomchik M. J., et. al., The role of B cells in lpr/lpr-induced autoimmunity. J. Exp Med. 180:1295-1306. 1994). Notably, an irreversible BTK inhibitor has been shown to display efficacy in the mouse MRL/lpr lupus model, reducing autoantibody production and renal damage (see Honigberg L. A., The Bruton tyrosine kinase inhibitor PCI-32765 blocks B-cell activation and is efficacious in models of autoimmune disease and B-cell malignancy. Proc. Natl. Acad. Sci. 107:13075-80. 2010).

There is also potential for BTK inhibitors for treating allergic diseases (see Honigberg, L., et. al., The selective BTK inhibitor PCI-32765 blocks B cell and mast cell activation and prevents mouse collagen induced arthritis. Clin. Immunol. 127 S1:S111. 2008). In addition, the irreversible inhibitor suppresses passive cutaneous anaphylaxis (PCA) induced by IgE antigen complex in mice (see Honigberg, L., et. al., The selective BTK inhibitor PCI-32765 blocks B cell and mast cell activation and prevents mouse collagen induced arthritis. Clin. Immunol. 127 S1:S111. 2008). These findings are in agreement with those noted with BTK-mutant mast cells and knockout mice and suggest that BTK inhibitors may be useful for the treatment of asthma, an IgE-dependent allergic disease of the airway.

In addition, platelet aggregation in response to collagen or collagen-related peptide is impaired in XLA patients who lack BTK function (see Quek L. S, et al., A role for Bruton's tyrosine kinase (BTK) in platelet activation by collagen. Curr. Biol. 8:1137-40. 1998). This is manifested by changes downstream from GPIV, such as phosphorylation of PLCgamma2 and calcium flux, which suggests potential utility in treating thromboembolic diseases.

Preclinical studies with a selective inhibitor of BTK have shown effects on spontaneous canine B cell lymphomas suggesting a potential utility in human lymphomas or other hematologic malignancies including chronic lymphocytic leukemia.

Accordingly, there is a need for compounds that inhibit tyrosine kinases thereby providing treatment for diseases such as autoimmune diseases, thromboembolic diseases and cancer. The present disclosure can fulfill this need and related needs.

In one aspect, this disclosure is directed to a compound of Formula (I′) or a pharmaceutically acceptable salt thereof:

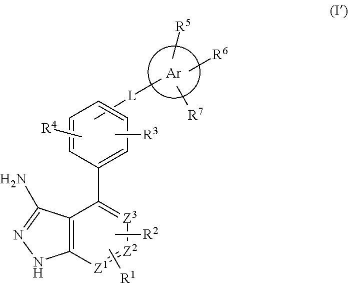

wherein:

Z¹, Z², and Z³ are —N— or CH, provided that not more than two of Z¹, Z², and Z³ are simultaneously N;

L is —O—, —C(O)—, —CH₂—, —S—, —S(O)—, —S(O₂)—, —N(R)—, —N(R)C(O)—, —C(O)N(R)—, —N(R′)S(O₂)—, —S(O₂)N(R′)—, or —N(R)C(O)N(R′)—, where each R and R′ is independently hydrogen, alkyl or cycloalkyl;

Ar is aryl, heteroaryl, cycloalkyl or heterocyclyl;

one of R¹ and R⁵ is hydrogen, alkyl, hydroxy, alkoxy, halo, haloalkyl, or haloalkoxy and the other of R¹ and R⁵ is:

(i) —P-Q-CH═C(R^{b})(EWG) where P is a bond, NR^{a} (where R^{a} is hydrogen or alkyl), —O—, —S—, —S(O)—, —S(O₂)—, alkylene or heteroalkylene, Q is a bond, aryl or heteroaryl wherein aryl or heteroaryl is optionally substituted with one or two substituents independently selected from hydrogen, halo, alkyl, alkoxy, alkylthio, haloalkyl, or haloalkoxy, R^{b} is cyano, nitro, halo, haloalkyl, haloalkoxy, alkylthio, or alkylsulfonyl and EWG is an electron withdrawing group; or

(ii) —Z-(EWG′)-C(R^{b})═CHR^{c} where Z is bond, NR^{a} (where R^{a} is hydrogen or alkyl), —O—, —S—, —S(O)—, —S(O₂)— alkylene, cycloalkylene, heteroalkylene, -(Z^{a})ₙ₁-aryl, or —(Z^{a})ₙ₁-heteroaryl (wherein n1 is 0 or 1, Z^{a} is NR^{a} (where R^{a} is hydrogen or alkyl), —O—, S, SO, SO₂, alkylene, or heteroalkylene and aryl or heteroaryl is optionally substituted with one or two substituents independently selected from hydrogen, halo, alkyl, alkoxy, alkylthio, haloalkyl, or haloalkoxy), EWG′ is a bond or an electron withdrawing group, R^{b} is cyano, nitro, halo, haloalkyl, haloalkoxy, alkylthio, or alkylsulfonyl and R^{c} is alkyl, substituted alkyl, haloalkoxy, cycloalkyl, cycloalkyleneNR^{d}R^{e} or cycloalkylene(alkylene)NR^{d}R^{e} (where R^{d} and R^{e} are independently hydrogen, alkyl, or cycloalkyl) or 3 to 6 membered saturated monocyclic heterocyclyl containing one or two heteroatoms selected from N, O, or S and optionally substituted with one or two substituents selected from hydroxy, alkyl or fluoro; or

(iii) a group of formula (a) or (b);

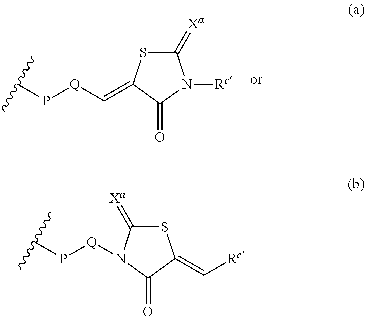

where P and Q are as defined above, X^{a} is O, S, or N(H or alkyl) and R^{c′} is hydrogen, alkyl, substituted alkyl, haloalkoxy, cycloalkyl, or cycloalkyleneNR^{d}R^{e} where R^{d} and R^{e} are independently hydrogen, alkyl, or cycloalkyl;

R² is hydrogen, alkyl, hydroxy, alkoxy, cyano, halo or haloalkyl;

R³ is hydrogen, alkyl, cycloalkyl, hydroxy, alkoxy, cyano, halo, haloalkyl or haloalkoxy;

R⁴ is hydrogen, alkyl, alkynyl, cycloalkyl, alkylamino, dialkylamino, alkylthio, alkylsulfonyl, carboxy, alkoxycarbonyl, alkylaminosulfonyl, dialkylaminosulfonyl, —CONH₂, alkylaminocarbonyl, dialkylaminocarbonyl, 3, 4, or 5 membered monocyclic heterocyclyl, hydroxy, alkoxy, cyano, halo, haloalkyl or haloalkoxy; and

R⁶ and R⁷ are independently hydrogen, alkyl, hydroxy, alkoxy, halo, haloalkyl, haloalkoxy, carboxy, alkoxycarbonyl, cyano, —CONH₂, amino, or monosubstituted and disubstituted amino.

In another aspect, the compound of Formula (I′) or a pharmaceutically acceptable salt thereof is a compound of Formula (I):

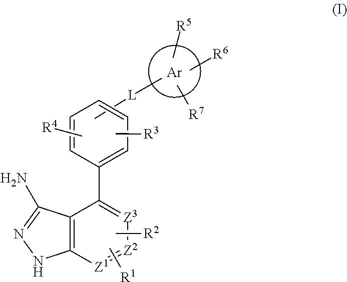

wherein:

Z¹, Z², and Z³ are —N— or CH, provided that not more than two of Z¹, Z², and Z³ are simultaneously N;

L is —O—, —C(O)—, —CH₂—, —S—, —S(O)—, —S(O₂)—, —N(R)—, —N(R)C(O)—, —C(O)N(R)—, —N(R′)S(O₂)—, —S(O₂)N(R′)—, or —N(R)C(O)N(R′)—, where each R and R′ is independently hydrogen, alkyl or cycloalkyl;

Ar is aryl, heteroaryl, cycloalkyl or heterocyclyl;

one of R¹ and R⁵ is hydrogen, alkyl, hydroxy, alkoxy, halo, haloalkyl, or haloalkoxy and the other of R¹ and R⁵ is:

(i) —P-Q-CH═C(R^{b})(EWG) where P is a bond, NR^{a} (where R^{a} is hydrogen or alkyl), —O—, —S—, —S(O)—, —S(O₂)—, alkylene or heteroalkylene, Q is a bond, aryl or heteroaryl wherein aryl or heteroaryl is optionally substituted with one or two substituents independently selected from hydrogen, halo, alkyl, alkoxy, alkylthio, haloalkyl, or haloalkoxy, R^{b} is cyano, nitro, halo, haloalkyl, haloalkoxy, alkylthio, or alkylsulfonyl and EWG is an electron withdrawing group; or

(ii) —Z-(EWG′)-C(R^{b})═CHR^{C} where Z is bond, NR^{a} (where R^{a} is hydrogen or alkyl), —O—, —S—, —S(O)—, —S(O₂)— alkylene, cycloalkylene, heteroalkylene, -(Z^{a})ₙ₁-aryl, or (Z^{a})ₙ₁-heteroaryl (wherein n1 is 0 or 1, Z^{a} is NR^{a} (where R^{a} is hydrogen or alkyl), —O—, S, SO, SO₂, alkylene, or heteroalkylene and aryl or heteroaryl is optionally substituted with one or two substituents independently selected from hydrogen, halo, alkyl, alkoxy, alkylthio, haloalkyl, or haloalkoxy), EWG′ is a bond or an electron withdrawing group, R^{b} is cyano, nitro, halo, haloalkyl, haloalkoxy, alkylthio, or alkylsulfonyl and R^{c} is alkyl, substituted alkyl, haloalkoxy, cycloalkyl, cycloalkyleneNR^{d}R^{e} where R^{d} and R^{e} are independently hydrogen, alkyl, or cycloalkyl; or

(iii) a group of formula (a) or (b);

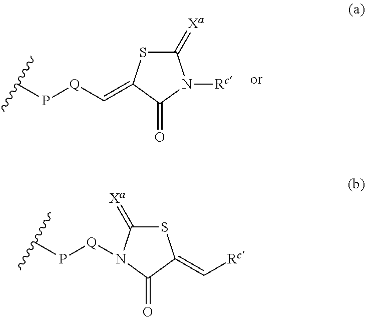

where P and Q are as defined above, X^{a} is O, S, or N(H or alkyl) and R^{c′} is hydrogen, alkyl, substituted alkyl, haloalkoxy, cycloalkyl, or cycloalkyleneNR^{d}R^{e} where R^{d} and R^{e} are independently hydrogen, alkyl, or cycloalkyl;

R² is hydrogen, alkyl, hydroxy, alkoxy, cyano, halo or haloalkyl;

R³ and R⁴ are independently hydrogen, alkyl, cycloalkyl, hydroxy, alkoxy, cyano, halo, haloalkyl or haloalkoxy; and

R⁶ and R⁷ are independently hydrogen, alkyl, hydroxy, alkoxy, halo, haloalkyl, haloalkoxy, carboxy, alkoxycarbonyl, cyano, —CONH₂, amino, monosubstituted and disubstituted amino.

In a second aspect, this disclosure is directed to a pharmaceutical composition comprising a compound of Formula (I′) or (I) (or any of the embodiments thereof described herein), or a pharmaceutically acceptable salt thereof; and a pharmaceutically acceptable excipient.

In a third aspect, this disclosure is directed to a method of treating a disease treatable by inhibition of a tyrosine kinase such as BLK, BMX, EGFR, HER2, HER4, ITK, JAK3, TEC, BTK, or TXK, preferably, BTK in a patient which method comprises administering to the patient in need thereof, a pharmaceutical composition comprising a compound of Formula (I′) or (I) or (or any of the embodiments thereof described herein) or a pharmaceutically acceptable salt thereof, and a pharmaceutically acceptable salt thereof and a pharmaceutically acceptable excipient. In one embodiment the disease is inflammatory disease such as arthritis, kidney disease, or cancer such as B-cell non-Hodgkin lymphoma.

In one embodiment of this aspect, the subject in need is suffering from an autoimmune disease, e.g., inflammatory bowel disease, arthritis, lupus, rheumatoid arthritis, psoriatic arthritis, osteoarthritis, Still's disease, juvenile arthritis, diabetes, myasthenia gravis, Hashimoto's thyroiditis, Ord's thyroiditis, Graves' disease, Sjogren's syndrome, multiple sclerosis, Guillain-Barre syndrome, acute disseminated encephalomyelitis, Addison's disease, opsoclonus-myoclonus syndrome, ankylosing spondylitisis, antiphospholipid antibody syndrome, aplastic anemia, autoimmune hepatitis, coeliac disease, Goodpasture's syndrome, idiopathic thrombocytopenic purpura, optic neuritis, scleroderma, primary biliary cirrhosis, Reiter's syndrome, Takayasu's arteritis, temporal arteritis, warm autoimmune hemolytic anemia, Wegener's granulomatosis, psoriasis, alopecia universalis, Behcet's disease, chronic fatigue, dysautonomia, endometriosis, interstitial cystitis, neuromyotonia, scleroderma, or vulvodynia. Preferably, the disease is rheumatoid arthritis. Preferably, the autoimmune disease is lupus.

In another embodiment of this aspect, the patient in need is suffering from a heteroimmune condition or disease, e.g., graft versus host disease, transplantation, transfusion, anaphylaxis, allergy, type I hypersensitivity, allergic conjunctivitis, allergic rhinitis, or atopic dermatitis.

In another embodiment of this aspect, the patient in need is suffering from an inflammatory disease, e.g., asthma, appendicitis, blepharitis, bronchiolitis, bronchitis, bursitis, cervicitis, cholangitis, cholecystitis, colitis, conjunctivitis, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, hepatitis, hidradenitis suppurativa, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, proctitis, prostatitis, pyelonephritis, rhinitis, salpingitis, sinusitis, stomatitis, synovitis, tendonitis, tonsillitis, uveitis, vaginitis, vasculitis, or vulvitis.

In another embodiment of this aspect, the patient is suffering from inflammatory skin disease which includes, by way of example, dermatitis, contact dermatitis, eczema, urticaria, rosacea, and scarring psoriatic lesions in the skin, joints, or other tissues or organs.

In yet another embodiment of this aspect, the subject in need is suffering from a cancer. In one embodiment, the cancer is a B-cell proliferative disorder, e.g., diffuse large B cell lymphoma, follicular lymphoma, chronic lymphocytic lymphoma, chronic lymphocytic leukemia, B-cell prolymphocytic leukemia, lymphoplamascytic lymphoma/Waldenstrom macroglobulinemia, splenic marginal zone lymphoma, plasma cell myeloma, plasmacytoma, extranodal marginal zone B cell lymphoma, nodal marginal zone B cell lymphoma, mantle cell lymphoma, mediastinal (thymic) large B cell lymphoma, intravascular large B cell lymphoma, primary effusion lymphoma, burkitt lymphoma/leukemia, or lymphomatoid granulomatosis. In some embodiments, the compound of Formula (I′) or (I) is administered in combination with another an anti-cancer agent e.g., the anti-cancer agent is an inhibitor of mitogen-activated protein kinase signaling, e.g., Tarceva®, Sutent®, Nexavar®, Tykerb®, Sprycel®, Gleevac®, U0126, PD98059, PD184352, PD0325901, ARRY-142886, SB239063, SP600125, BAY 43-9006, wortmannin, LY294002, Nexavar®, Tarceva®, Sutent®, Tykerb®, Sprycel®, Crizotinib, and Xalkori®.

In yet another embodiment, the patient in need is suffering from a thromboembolic disorder, e.g., myocardial infarct, angina pectoris, reocclusion after angioplasty, restenosis after angioplasty, reocclusion after aortocoronary bypass, restenosis after aortocoronary bypass, stroke, transitory ischemia, a peripheral arterial occlusive disorder, pulmonary embolism, or deep venous thrombosis.

In a fourth aspect, the disclosure is directed to use of compound of Formula (I′) or (I) (and any embodiments thereof described herein) for use as a medicament. In one embodiment, the use of compound of Formula (I′) or (I) is for treating inflammatory disease or proliferative diseases.

In a fifth aspect is the use of a compound of Formula (I′) or (I) in the manufacture of a medicament for treating an inflammatory disease in a patient in which the activity of BTK or other tyrosine kinases such as BLK, BMX, EGFR, HER2, HER4, ITK, JAK3, TEC, or TXK contributes to the pathology and/or symptoms of the disease. In one embodiment of this aspect, the tyrosine kinase protein is BTK. In another embodiment of this aspect, the inflammatory disease is respiratory, cardiovascular, or proliferative diseases.

In any of the aforementioned aspects involving the treatment of proliferative disorders, including cancer, are further embodiments comprising administering the compound of Formula (I′) or (I) in combination with at least one additional agent selected from the group consisting of alemtuzumab, arsenic trioxide, asparaginase (pegylated or non-), bevacizumab, cetuximab, platinum-based compounds such as cisplatin, cladribine, daunorubicin/doxorubicin/idarubicin, irinotecan, fludarabine, 5-fluorouracil, gemtuzamab, methotrexate, paclitaxel, Taxol™, temozolomide, thioguanine, or classes of drugs including hormones (an antiestrogen, an antiandrogen, or gonadotropin releasing hormone analogues, interferons such as alpha interferon, nitrogen mustards such as busulfan or melphalan or mechlorethamine, retinoids such as tretinoin, topoisomerase inhibitors such as irinotecan or topotecan, tyrosine kinase inhibitors such as gefinitinib or imatinib, or agents to treat signs or symptoms induced by such therapy including allopurinol, filgrastim, granisetron/ondansetron/palonosetron, dronabinol. When combination therapy is used, the agents can be administered simultaneously or sequentially.

In a sixth aspect is directed to an intermediate of Formula (II):

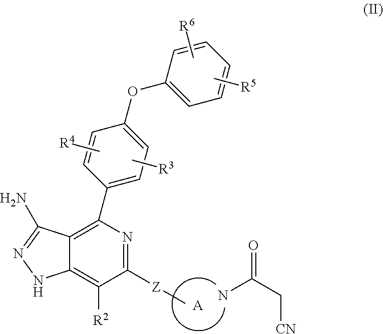

wherein:

R² is hydrogen or alkyl;

R³ and R⁴ are independently hydrogen, alkyl, haloalkyl, fluoro or chloro;

R⁵ and R⁶ are independently hydrogen or fluoro;

Z is a bond or alkylene;

ring A

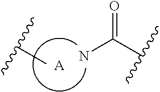

in is heterocycloamino optionally substituted with one or two alkyl;
or a salt thereof.

Preferably,

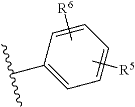

is a ring of formula:

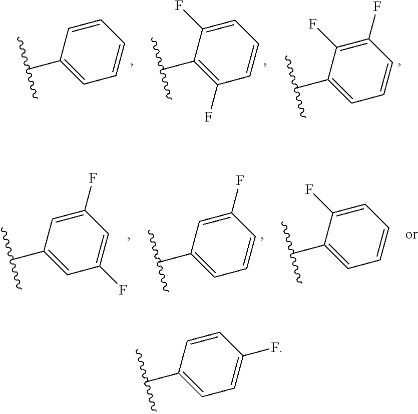

### Preferably,

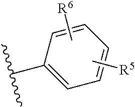

is a ring of formula:

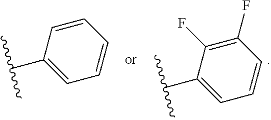 [SMILES: *c1ccccc1 ; *c1cccc(F)c1F]

Preferably, R² is hydrogen or alkyl, more preferably hydrogen.

R³ and R⁴ are independently hydrogen, methyl, ethyl, trifluoromethyl, fluoro or chloro. Preferably,

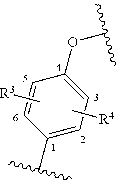

is a ring of formula:

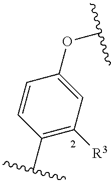

where R³ is hydrogen, methyl, ethyl, chloro, fluoro or trifluoromethyl, preferably hydrogen, methyl, ethyl, chloro or fluoro, more preferably, hydrogen, methyl, fluoro, or chloro, even more preferably hydrogen, chloro or fluoro, particularly preferably hydrogen or fluoro. Preferably,

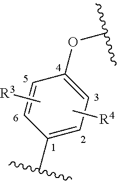

is a ring of formula

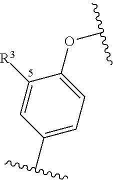

where R³ is alkyl or halo, preferably methyl, chloro or fluoro.

Preferably,

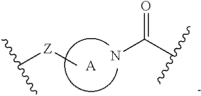

is 3-piperidin-1-carbonyl (ie, the C-3 carbon of the piperidin-1-yl ring is attached to the C6 position of 1H-pyrazolo[4,3-c]pyridin-3-amine ring) or 2-CH₂-pyrrolidin-1-ylcarbonyl, 2-CH(CH₃)-pyrrolidin-1-ylcarbonyl; 2-CH₂-3,3-dimethylpyrrolidin-1-ylcarbonyl or 2-CH₂-4,4-dimethylpyrrolidin-1-ylcarbonyl, the carbon atom of the pyrrolidinyl ring attached to —CH₂— having (R) or (S) stereochemistry. More preferably,

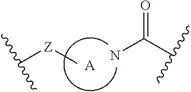

is 2-CH₂-pyrrolidin-1-ylcarbonyl (ie, the 2-CH₂-pyrrolidin-1-ylcarbonyl ring is attached via the CH₂ group located at C2 position of the pyrrolidinyl ring to the C6 position of 1H-pyrazolo[4,3-c]pyridin-3-amine ring). More preferably,

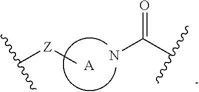

is 3-piperidin-1-carbonyl and the stereochemistry at the C-3 carbon of the piperidin-1-ylcarbonyl ring is (R).

### DEFINITIONS

Unless otherwise stated, the following terms used in the specification and claims are defined for the purposes of this Application and have the following meaning:

“Alkyl” means a linear saturated monovalent hydrocarbon radical of one to six carbon atoms or a branched saturated monovalent hydrocarbon radical of three to six carbon atoms, e.g., methyl, ethyl, propyl, 2-propyl, butyl (including all isomeric forms), pentyl (including all isomeric forms), and the like.

“Alkylene” means a linear saturated divalent hydrocarbon radical of one to six carbon atoms or a branched saturated divalent hydrocarbon radical of three to six carbon atoms unless otherwise stated e.g., methylene, ethylene, propylene, 1-methylpropylene, 2-methylpropylene, butylene, pentylene, and the like.

“Alkylthio” means a —SR radical where R is alkyl as defined above, e.g., methylthio, ethylthio, and the like.

“Alkylsulfonyl” means a —SO₂R radical where R is alkyl as defined above, e.g., methylsulfonyl, ethylsulfonyl, and the like.

“Amino” means a —NH₂.

“Alkylamino” means a —NHR radical where R is alkyl as defined above, e.g., methylamino, ethylamino, propylamino, or 2-propylamino, and the like.

“Alkoxy” means a —OR radical where R is alkyl as defined above, e.g., methoxy, ethoxy, propoxy, or 2-propoxy, n-, iso-, or tert-butoxy, and the like.

“Alkoxyalkyl” means a linear monovalent hydrocarbon radical of one to six carbon atoms or a branched monovalent hydrocarbon radical of three to six carbons substituted with at least one alkoxy group, preferably one or two alkoxy groups, as defined above, e.g., 2-methoxyethyl, 1-, 2-, or 3-methoxypropyl, 2-ethoxyethyl, and the like.

“Alkoxycarbonyl” means a —C(O)OR radical where R is alkyl as defined above, e.g., methoxycarbonyl, ethoxycarbonyl, and the like.

“Aminocarbonyl” means a —CONRR′ radical where R is independently hydrogen, alkyl, or substituted alkyl, each as defined herein and R′ is hydrogen, alkyl, cycloalkyl, cycloalkylalkyl, aryl, aralkyl, heteroaryl, heteroaralkyl, heterocyclyl, heterocyclylalkyl, or substituted alkyl, each as defined herein, and wherein the aryl, heteroaryl, or heterocyclyl ring either alone or part of another group e.g., aralkyl, is optionally substituted with one, two, or three substituents independently selected from alkyl, alkoxy, halo, haloalkoxy, hydroxyl, carboxy, or alkoxycarbonyl, e.g., —CONH₂, methylaminocarbonyl, 2-dimethylaminocarbonyl, and the like. When R is hydrogen and R′ is alkyl in —CONRR′, the group is also referred to herein as alkylaminocarbonyl and when R and R′ are both alkyl in —CONRR′, the group is also referred to herein as dialkylaminocarbonyl.

“Aminosulfonyl” means a —SO₂NRR′ radical where R is independently hydrogen, alkyl, or substituted alkyl, each as defined herein and R′ is hydrogen, alkyl, cycloalkyl, cycloalkylalkyl, aryl, aralkyl, heteroaryl, heteroaralkyl, heterocyclyl, heterocyclylalkyl, or substituted alkyl, each as defined herein, and wherein the aryl, heteroaryl, or heterocyclyl ring either alone or part of another group e.g., aralkyl, is optionally substituted with one, two, or three substituents independently selected from alkyl, alkoxy, halo, haloalkoxy, hydroxyl, carboxy, or alkoxycarbonyl, e.g., —SO₂NH₂, methylaminosulfonyl, dimethylaminosulfonyl, and the like. When R is hydrogen and R′ is alkyl in —SO₂NRR′, the group is also referred to herein as alkylaminosulfonyl and when R and R′ are both alkyl in —CONRR′, the group is also referred to herein as dialkylaminosulfonyl. “Acyl” means a —COR radical where R is alkyl, haloalkyl, cycloalkyl, cycloalkylalkyl, aryl, aralkyl, heteroaryl, heteroaralkyl, heterocyclyl, or heterocyclylalkyl, each as defined herein, and wherein the aryl, heteroaryl, or heterocyclyl ring either alone or part of another group e.g., aralkyl, is optionally substituted with one, two, or three substituents independently selected from alkyl, alkoxy, halo, haloalkoxy, hydroxyl, carboxy, or alkoxycarbonyl, e.g., acetyl, propionyl, benzoyl, pyridinylcarbonyl, and the like. When R is alkyl, the radical is also referred to herein as alkylcarbonyl.

“Aryl” means a monovalent monocyclic or bicyclic aromatic hydrocarbon radical of 6 to 10 ring atoms e.g., phenyl or naphthyl.

“Aralkyl” means a -(alkylene)-R radical where R is aryl as defined above.

“Cycloalkyl” means a cyclic saturated monovalent hydrocarbon radical of three to ten carbon atoms wherein one or two carbon atoms may be replaced by an oxo group, e.g., cyclopropyl, cyclobutyl, cyclopentyl, or cyclohexyl, and the like.

“Cycloalkylalkyl” means a -(alkylene)-R radical where R is cycloalkyl as defined above; e.g., cyclopropylmethyl, cyclobutylmethyl, cyclopentylethyl, or cyclohexylmethyl, and the like.

“Cycloalkylene” means a divalent cyclic saturated hydrocarbon radical of three to ten carbon atoms wherein one or two carbon atoms may be replaced by an oxo group, e.g., cyclopropylene, cyclobutylene, cyclopentylene, or cyclohexylene, and the like.

“Carboxy” means —COOH.

“Disubstituted amino” means a —NRR′ radical where R and R′ are independently alkyl, cycloalkyl, cycloalkylalkyl, acyl, sulfonyl, aryl, aralkyl, heteroaryl, heteroaralkyl, heterocyclyl, heterocyclylalkyl, or substituted alkyl, each as defined herein, and wherein the aryl, heteroaryl, or heterocyclyl ring either alone or part of another group e.g., aralkyl, is optionally substituted with one, two, or three substituents independently selected from alkyl, alkoxy, halo, haloalkoxy, hydroxyl, carboxy, or alkoxycarbonyl, e.g., dimethylamino, phenylmethylamino, and the like. When the R and R′ groups are alkyl, the disubstituted amino group maybe referred to herein as dialkylamino.

The term “electron withdrawing group” refers to a chemical substituent that modifies the electrostatic forces acting on a nearby chemical reaction center by withdrawing negative charge from that chemical reaction center. Thus, electron withdrawing groups draw electrons away from a reaction center. As a result, the reaction center is fractionally more positive than it would be in the absence of the electron-withdrawing group. In some embodiments, the chemical reaction center is one of the two carbons forming the carbon-carbon double bond (olefin). In some embodiments, the chemical reaction center is the olefin carbon attached to EWG. The electron withdrawing group functions to draw charge or electrons away from this olefin carbon thereby making the olefin carbon electron deficient (relative to the absence of the electron withdrawing group). The electron deficient olefin carbon is thereby rendered more reactive toward electron rich chemical groups, such as the sulfhydryl of a kinase active site cysteine.

Some non-limiting examples of EWG include, but are not limited to, —N(R′₂), —N(R′₃)⁺, —SO₃H, —SO₃R′, —S(O₂)R′, —S(O)R′, —C(O)NH₂, —C(O)NHR^{g}, —C(O)NR^{f}R^{g}, —S(O₂)NH₂, —SO₂NHRⁱ′, —SO₂NR^{h}Rⁱ, —PO(OR′)₂, —PO₃H₂, —PO(NR′₂)₂, —C≡N, —CH(haloalkyl), —C(O)X′, —COOH, —COOR′, —C(O)R′, —C(O)H, —P(O)(OR′)OR″, halo, heteroaryl, or aryl; wherein X′ is independently halogen (e.g. chloro or fluoro), R′, R″, R^{f}, R^{g}, R^{h}, and Rⁱ are independently hydrogen, alkyl, substituted alkyl, cycloalkyl, cycloalkyleneNR^{d}R^{e} (where R^{d} and R^{e} are independently hydrogen, alkyl, or cycloalkyl) or R^{f} and R^{g} and R^{h} and Rⁱ together with the nitrogen atom to which they are attached form heterocycloamino; wherein each of the aforementioned ring is substituted with one, two or three substituents independently selected from hydrogen, alkyl, alkoxy, hydroxyl, cyano, nitro, halo, haloalkyl, haloalkoxy, alkylthio, alkylsulfonyl, carboxy, alkoxycarbonyl, aminocarbonyl or aminosulfonyl.

Preferably, EWG is —CO—NR^{f}R^{g}, —SO₂NR^{h}Rⁱ (wherein R^{f} and R^{h} are independently hydrogen, alkyl, or cycloalkyl and R^{g} and Rⁱ are independently hydrogen, alkyl, substituted alkyl, or cycloalkyleneNR^{d}R^{e} (where R^{d} and R^{e} are independently hydrogen, alkyl, or cycloalkyl); or R^{f} and R^{g} and R^{h} and Rⁱ together with the nitrogen atom to which they are attached form heterocycloamino), aryl or heteroaryl wherein each of the aforementioned ring is substituted with one, two or three substituents independently selected from hydrogen, alkyl, alkoxy, hydroxyl, cyano, nitro, halo, haloalkyl, haloalkoxy, alkylthio, alkylsulfonyl, carboxy, alkoxycarbonyl, aminocarbonyl or aminosulfonyl. Preferably, the heteroaryl ring is pyridinyl, pyrazolyl, indazolyl, indolyl, thienyl, pyrrolyl, imidazolyl, thiazolyl, benzothiazolyl, oxazolyl, benzimidazolyl, benzoxazolyl, isoxazolyl, benzisoxazolyl, triazolyl, benzotriazolyl, quinolinyl, isoquinolinyl, quinazolinyl, pyrimidinyl, or pyridinyl N-oxide optionally substituted as defined in previous paragraph.

Some non-limiting examples of EWG′ include, but are not limited to, —CH(haloalkyl), —NR′—, —S(O₂)—, —S(O)—, —CO—, —NR′CO—, —NR′SO₂—, —PO(OR′)—,

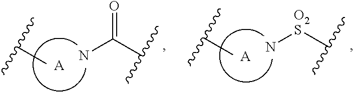

heteroaryl, or aryl; wherein each R′ is independently hydrogen, alkyl, substituted alkyl, cycloalkyl; ring A is heterocycloamino where the carbonyl and sulfonyl groups are attached to —C(R^{b})═CHR^{c} in the definition of R¹ and R⁵ in compound of Formula (I′) or (I); and heterocycloamino, aryl and heteroaryl are substituted with one, two or three substituents independently selected from hydrogen, alkyl, alkoxy, hydroxyl, cyano, nitro, halo, haloalkyl, haloalkoxy, alkylthio, alkylsulfonyl, carboxy, alkoxycarbonyl, aminocarbonyl or aminosulfonyl. Preferably, the heteroaryl ring is pyridinyl, pyrazolyl, indazolyl, indolyl, thienyl, pyrrolyl, imidazolyl, thiazolyl, benzothiazolyl, oxazolyl, benzimidazolyl, benzoxazolyl, isoxazolyl, benzisoxazolyl, triazolyl, benzotriazolyl, quinolinyl, isoquinolinyl, quinazolinyl, pyrimidinyl, or pyridinyl N-oxide optionally substituted as defined in previous paragraph. In the groups above, the left side of the group is attached to Z and right side is attached to —C(R^{b})═CHR^{c} e.g., in —NR′CO—, NR′ is attached to Z and CO is attached to —C(R^{b})═CHR^{c}.

In some embodiments, a composition of the present disclosure comprises a compound corresponding to Formula (I′) or (I) (or a pharmaceutically acceptable salt thereof) in which R¹ or R⁵ is —Z-(EWG′)-C(R^{b})═CHR^{c} group, Z is a bond, and the ring in the compound of Formula (I′) or (I) to which R¹ is attached, i.e.,

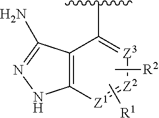

or the Ar ring to which R⁵ is attached, respectively, possesses an electron deficient π system. In such embodiments, Z and EWG′ may each be bonds and the —C(R^{b})═CHR^{c} group is directly attached to the Ar or

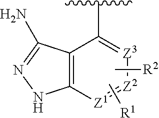

ring in the compound of Formula (I′) or (I). In general, a ring has an electron deficient n system when it is substituted with an electron withdrawing group or the ring itself is electron deficient, e.g., a heteroaryl ring containing electronegative ring atoms such as nitrogen, oxygen or sulfur. For example, in the compounds of Formula (I), when Ar is phenyl, the phenyl ring can be electron deficient when it is substituted with an electron withdrawing group such as halo, cyano, or haloalkyl. By way of further example, the Ar ring can also be an electron deficient n system when it is heteroaryl, e.g., one of

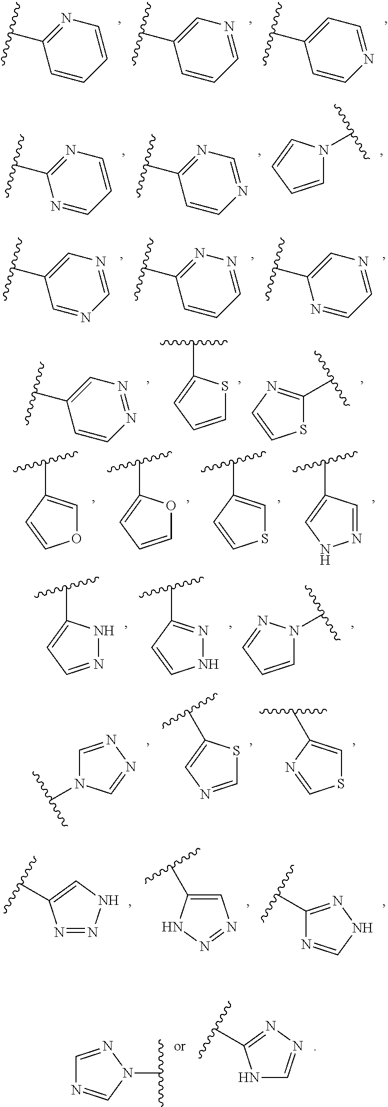

optionally substituted as defined above.

“Halo” means fluoro, chloro, bromo, or iodo, preferably fluoro or chloro.

“Haloalkyl” means alkyl radical as defined above, which is substituted with one or more halogen atoms, preferably one to five halogen atoms, preferably fluorine or chlorine, including those substituted with different halogens, e.g., —CH₂Cl, —CF₃, —CHF₂, —CH₂CF₃, —CF₂CF₃, —CF(CH₃)₂, and the like. When the alkyl is substituted with only fluoro, it is referred to in this Application as fluoroalkyl.

“Haloalkoxy” means a —OR radical where R is haloalkyl as defined above e.g., —OCF₃, —OCHF₂, and the like. When R is haloalkyl where the alkyl is substituted with only fluoro, it is referred to in this Application as fluoroalkoxy.

“Hydroxyalkyl” means a linear monovalent hydrocarbon radical of one to six carbon atoms or a branched monovalent hydrocarbon radical of three to six carbons substituted with one or two hydroxy groups, provided that if two hydroxy groups are present they are not both on the same carbon atom. Representative examples include, but are not limited to, hydroxymethyl, 2-hydroxyethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl, preferably 2-hydroxyethyl, 2,3-dihydroxypropyl, and 1-(hydroxymethyl)-2-hydroxyethyl.

“Heterocyclyl” means a saturated or unsaturated monovalent monocyclic group of 4 to 8 ring atoms in which one or two ring atoms are heteroatom selected from N, O, or S(O)ₙ, where n is an integer from 0 to 2, the remaining ring atoms being C. The heterocyclyl ring is optionally fused to a (one) aryl or heteroaryl ring as defined herein provided the aryl and heteroaryl rings are monocyclic. The heterocyclyl ring fused to monocyclic aryl or heteroaryl ring is also referred to in this Application as “bicyclic heterocyclyl” ring. Additionally, one or two ring carbon atoms in the heterocyclyl ring can optionally be replaced by a —CO— group. More specifically the term heterocyclyl includes, but is not limited to, pyrrolidino, piperidino, homopiperidino, 2-oxopyrrolidinyl, 2-oxopiperidinyl, morpholino, piperazino, tetrahydropyranyl, thiomorpholino, and the like. When the heterocyclyl ring is unsaturated it can contain one or two ring double bonds provided that the ring is not aromatic. When the heterocyclyl group contains at least one nitrogen atom, it is also referred to herein as heterocycloamino and is a subset of the heterocyclyl group. When the heterocyclyl group is a saturated ring and is not fused to aryl or heteroaryl ring as stated above, it is also referred to herein as saturated monocyclic heterocyclyl.

“Heterocyclylalkyl” means a -(alkylene)-R radical where R is heterocyclyl ring as defined above e.g., tetraydrofuranylmethyl, piperazinylmethyl, morpholinylethyl, and the like.

“Heterocycloamino” means a saturated or unsaturated monovalent monocyclic group of 4 to 8 ring atoms in which one or two ring atoms are heteroatom selected from N, O, or S(O)ₙ, where n is an integer from 0 to 2, the remaining ring atoms being C provided that at least one of the ring atoms is N. Additionally, one or two ring carbon atoms in the heterocycloamino ring can optionally be replaced by a —CO— group. When the heterocyclyl ring is unsaturated it can contain one or two ring double bonds provided that the ring is not aromatic. Unless otherwise stated, the heterocyloamino ring can optionally be substituted with one, two, or three substituents independently selected from alkyl, hydroxyl, alkoxy, amino, alkylamino, or dialkylamino.

“Heteroaryl” means a monovalent monocyclic or bicyclic aromatic radical of 5 to 10 ring atoms where one or more, preferably one, two, or three, ring atoms are heteroatom selected from N, O, or S, the remaining ring atoms being carbon. Representative examples include, but are not limited to, pyrrolyl, thienyl, thiazolyl, imidazolyl, furanyl, indolyl, isoindolyl, oxazolyl, isoxazolyl, benzothiazolyl, benzoxazolyl, quinolinyl, isoquinolinyl, pyridinyl, pyrimidinyl, pyrazinyl, pyridazinyl, triazolyl, tetrazolyl, and the like.

“Heteroalkylene” means a -(alkylene)-radical where one, two or three carbons in the alkylene chain is replaced by —O—, N(H, alkyl, or substituted alkyl), S, SO, SO₂, or CO.

“Heteroaralkyl” means an -alkylene- radical where R is heteroaryl as defined above.

“Monosubstituted amino” means a —NHR radical where R is alkyl, cycloalkyl, cycloalkylalkyl, acyl, sulfonyl, aryl, aralkyl, heteroaryl, heteroaralkyl, heterocyclyl, heterocyclylalkyl, or substituted alkyl, each as defined herein, and wherein the aryl, heteroaryl, or heterocyclyl ring either alone or part of another group e.g., aralkyl, is optionally substituted with one, two, or three substituents independently selected from alkyl, alkoxy, halo, haloalkoxy, hydroxyl, carboxy, or alkoxycarbonyl, e.g., methylamino, phenylamino, hydroxyethylamino, and the like. When R is alkyl, the monosubstituted amino group maybe referred to herein as alkylamino. The present disclosure also includes the prodrugs of compounds of Formula (I′) or (I). The term prodrug is intended to represent covalently bonded carriers, which are capable of releasing the active ingredient of Formula (I′) or (I) when the prodrug is administered to a mammalian subject. Release of the active ingredient occurs in vivo. Prodrugs can be prepared by techniques known to one skilled in the art. These techniques generally modify appropriate functional groups in a given compound. These modified functional groups however regenerate original functional groups in vivo or by routine manipulation. Prodrugs of compounds of Formula (I′) or (I) include compounds wherein a hydroxy, amino, carboxylic, or a similar group is modified. Examples of prodrugs include, but are not limited to esters (e.g., acetate, formate, and benzoate derivatives), carbamates (e.g., N,N-dimethylaminocarbonyl) of hydroxy or amino functional groups in compounds of Formula (I′) or (I), amides (e.g., trifluoroacetylamino, acetylamino, and the like), and the like. Prodrugs of compounds of Formula (I′) or (I) are also within the scope of this disclosure.

The present disclosure also includes protected derivatives of compounds of Formula (I′) or (I). For example, when compounds of Formula (I′) or (I) contain groups such as hydroxy, carboxy, thiol or any group containing a nitrogen atom(s), these groups can be protected with a suitable protecting groups. A comprehensive list of suitable protective groups can be found in T. W. Greene, Protective Groups in Organic Synthesis, John Wiley & Sons, Inc. (1999), the disclosure of which is incorporated herein by reference in its entirety. The protected derivatives of compounds of Formula (I′) or (I) can be prepared by methods well known in the art.

The present disclosure also includes polymorphic forms (amorphous as well as crystalline) and deuterated forms of compounds of Formula (I′) or (I).

A “pharmaceutically acceptable salt” of a compound means a salt that is pharmaceutically acceptable and that possesses the desired pharmacological activity of the parent compound. Such salts include:

acid addition salts, formed with inorganic acids such as hydrochloric acid, hydrobromic acid, sulfuric acid, nitric acid, phosphoric acid, and the like; or formed with organic acids such as formic acid, acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or

salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

The compounds of the present disclosure may have asymmetric centers. Compounds of the present disclosure containing an asymmetrically substituted atom may be isolated in optically active or racemic forms. It is well known in the art how to prepare optically active forms, such as by resolution of materials. All chiral, diastereomeric, racemic forms are within the scope of this disclosure, unless the specific stereochemistry or isomeric form is specifically indicated.

Certain compounds of Formula (I′) or (I) can exist as tautomers and/or geometric isomers. All possible tautomers and cis and trans isomers, as individual forms and mixtures thereof are within the scope of this disclosure. Additionally, as used herein the term alkyl includes all the possible isomeric forms of said alkyl group albeit only a few examples are set forth. Furthermore, when the cyclic groups such as aryl, heteroaryl, heterocyclyl are substituted, they include all the positional isomers albeit only a few examples are set forth. Furthermore, all polymorphic forms and hydrates of a compound of Formula (I′) or (I) are within the scope of this disclosure.

“Oxo” or “carbonyl” means ═(O) group.

“Optional” or “optionally” means that the subsequently described event or circumstance may but need not occur, and that the description includes instances where the event or circumstance occurs and instances in which it does not. For example, “heterocyclyl group optionally substituted with an alkyl group” means that the alkyl may but need not be present, and the description includes situations where the heterocyclyl group is substituted with an alkyl group and situations where the heterocyclyl group is not substituted with alkyl.

A “pharmaceutically acceptable carrier or excipient” means a carrier or an excipient that is useful in preparing a pharmaceutical composition that is generally safe, non-toxic and neither biologically nor otherwise undesirable, and includes a carrier or an excipient that is acceptable for veterinary use as well as human pharmaceutical use. “A pharmaceutically acceptable carrier/excipient” as used in the specification and claims includes both one and more than one such excipient.

“Sulfonyl” means a —SO₂R radical where R is alkyl, haloalkyl, aryl, aralkyl, heteroaryl, heteroaralkyl, heterocyclyl, heterocyclylalkyl, each as defined herein, and wherein the aryl, heteroaryl, or heterocyclyl ring either alone or part of another group e.g., aralkyl, is optionally substituted with one, two, or three substituents independently selected from alkyl, alkoxy, halo, haloalkoxy, hydroxyl, carboxy, or alkoxycarbonyl, e.g., methylsulfonyl, phenylsulfonyl, benzylsulfonyl, pyridinylsulfonyl, and the like.

“Substituted alkyl” means alkyl group as defined herein which is substituted with one, two, or three substituents independently selected from hydroxyl, alkoxy, carboxy, cyano, alkoxycarbonyl, alkylthio, alkylsulfonyl, halo, haloalkoxy, —CONRR′ or —NRR′ (where each R is hydrogen, alkyl, hydroxyalkyl, or alkoxyalkyl, and each R′ is hydrogen, alkyl, or cycloalkyl) or heterocyclyl (preferably heterocycloamino) optionally substituted with one or two groups independently selected from alkyl, hydroxyl, alkoxy, alkylthio, alkylsulfonyl, halo, or —CONRR′ where R and R; are as defined above.

“Treating” or “treatment” of a disease includes:

(1) preventing the disease, i.e. causing the clinical symptoms of the disease not to develop in a mammal that may be exposed to or predisposed to the disease but does not yet experience or display symptoms of the disease;

(2) inhibiting the disease, i.e., arresting or reducing the development of the disease or its clinical symptoms; or

(3) relieving the disease, i.e., causing regression of the disease or its clinical symptoms.

A “therapeutically effective amount” means the amount of a compound of Formula (I′) or (I) that, when administered to a mammal for treating a disease, is sufficient to effect such treatment for the disease. The “therapeutically effective amount” will vary depending on the compound, the disease and its severity and the age, weight, etc., of the mammal to be treated.

The abbreviations appearing in the Embodiment E of the embodiments section shall have the following meanings:

I(a), I(b), I(c), I(d), and I(e) shall mean a compound corresponding to Formula (I) having the substituents described in subpart (a), subpart (b), subpart (d) and subpart (e), respectively, of Embodiment E.

A(a), A(b), A(c), A(d), and A(e) shall mean a compound corresponding to Formula (I) having the substituents described in Embodiment A and subpart (a), subpart (b), subpart (d) and subpart (e), respectively, of Embodiment E.

B(a), B(b), B(c), B(d), and B(e) shall mean a compound corresponding to Formula (I) having the substituents described in Embodiment B and subpart (a), subpart (b), subpart (d) and subpart (e), respectively, of Embodiment E.

C(a), C(b), C(c), C(d), and C(e) shall mean a compound corresponding to Formula (I) having the substituents described in Embodiment C and subpart (a), subpart (b), subpart (d) and subpart (e), respectively, of Embodiment E.

D(a), D(b), D(c), D(d), and D(e) shall mean a compound corresponding to Formula (I) having the substituents described in Embodiment D and subpart (a), subpart (b), subpart (d) and subpart (d), respectively, of Embodiment E.

I(a,c), I(a,d), I(a,e), I(b,c), I(b,d), and I(b,e) shall mean a compound corresponding to Formula (I) having the substituents described in subparts (a) and (c), subparts (a) and (d), subparts (a) and (e), subparts (b) and (c), subparts (b) and (d) and subparts (b) and (e), respectively, of Embodiment E.

A(a,c), A(a,d), A(a,e), A(b,c), A(b,d), and A(b,e) shall mean a compound corresponding to Formula (I) having the substituents described in Embodiment A and subparts (a) and (c), subparts (a) and (d), subparts (a) and (e), subparts (b) and (c), subparts (b) and (d) and subparts (b) and (e), respectively, of Embodiment E.

B(a,c), B(a,d), B(a,e), B(b,c), B(b,d), and B(b,e) shall mean a compound corresponding to Formula (I) having the substituents described in Embodiment B and subparts (a) and (c), subparts (a) and (d), subparts (a) and (e), subparts (b) and (c), subparts (b) and (d) and subparts (b) and (e), respectively, of Embodiment E.

C(a,c), C(a,d), C(a,e), C(b,c), C(b,d), and C(b,e) shall mean a compound corresponding to Formula (I) having the substituents described in Embodiment C and subparts (a) and (c), subparts (a) and (d), subparts (a) and (e), subparts (b) and (c), subparts (b) and (d) and subparts (b) and (e), respectively, of Embodiment E.

D(a,c), D(a,d), D(a,e), D(b,c), D(b,d), and D(b,e) shall mean a compound corresponding to Formula (I) having the substituents described in Embodiment D and subparts (a) and (c), subparts (a) and (d), subparts (a) and (e), subparts (b) and (c), subparts (b) and (d) and subparts (b) and (e), respectively, of Embodiment E.

I(a,i), I(b,i), I(c,i), I(d,i), and I(e,i) shall mean a compound corresponding to Formula (I) having the substituents described subparts (a) and (i), subparts (b) and (i), subparts (c) and (i), subparts (d) and (i), and subparts (e) and (i), respectively, of Embodiment E.

A(a,i), A(b,i), A(c,i), A(d,i), and A(e,i) shall mean a compound corresponding to Formula (I) having the substituents described in Embodiment A and subparts (a) and (i), subparts (b) and (i), subparts (c) and (i), subparts (d) and (i), and subparts (e) and (i), respectively, of Embodiment E.

B(a,i), B(b,i), B(c,i), B(d,i), and B(e,i) shall mean a compound corresponding to Formula (I) having the substituents described in Embodiment B and subparts (a) and (i), subparts (b) and (i), subparts (c) and (i), subparts (d) and (i), and subparts (e) and (i), respectively, of Embodiment E.

C(a,i), C(b,i), C(c,i), C(d,i), and C(e,i) shall mean a compound corresponding to Formula (I) having the substituents described in Embodiment C and subparts (a) and (i), subparts (b) and (i), subparts (c) and (i), subparts (d) and (i), and subparts (e) and (i), respectively, of Embodiment E.

D(a,i), D(b,i), D(c,i), D(d,i), and D(e,i) shall mean a compound corresponding to Formula (I) having the substituents described in Embodiment D and subparts (a) and (i), subparts (b) and (i), subparts (c) and (i), subparts (d) and (i), and subparts (e) and (i), respectively, of Embodiment E.

I(a,c,i), I(a,d,i), I(a,e,i), I(b,c,i), I(b,d,i), and I(b,e,i) shall mean a compound corresponding to Formula (I) having the substituents described in subparts (a), (c) and (i), subparts (a), (d) and (i), subparts (a), (e) and (i), subparts (b), (c) and (i), subparts (b), (d) and (i) and subparts (b), (e) and (i), respectively, of Embodiment E.

A(a,c,i), A(a,d,i), A(a,e,i), A(b,c,i), A(b,d,i), and A(b,e,i) shall mean a compound corresponding to Formula (I) having the substituents described in Embodiment A and subparts (a), (c) and (i), subparts (a), (d) and (i), subparts (a), (e) and (i), subparts (b), (c) and (i), subparts (b), (d) and (i) and subparts (b), (e) and (i), respectively, of Embodiment E.

B(a,c,i), B(a,d,i), B(a,e,i), B(b,c,i), B(b,d,i), and B(b,e,i) shall mean a compound corresponding to Formula (I) having the substituents described in Embodiment B and subparts (a), (c) and (i), subparts (a), (d) and (i), subparts (a), (e) and (i), subparts (b), (c) and (i), subparts (b), (d) and (i) and subparts (b), (e) and (i), respectively, of Embodiment E.

C(a,c,i), C(a,d,i), C(a,e,i), C(b,c,i), C(b,d,i), and C(b,e,i) shall mean a compound corresponding to Formula (I) having the substituents described in Embodiment C and subparts (a), (c) and (i), subparts (a), (d) and (i), subparts (a), (e) and (i), subparts (b), (c) and (i), subparts (b), (d) and (i) and subparts (b), (e) and (i), respectively, of Embodiment E.

D(a,c,i), D(a,d,i), D(a,e,i), D(b,c,i), D(b,d,i), and D(b,e,i) shall mean a compound corresponding to Formula (I) having the substituents described in Embodiment D and subparts (a), (c) and (i), subparts (a), (d) and (i), subparts (a), (e) and (i), subparts (b), (c) and (i), subparts (b), (d) and (i) and subparts (b), (e) and (i), respectively, of Embodiment E., I(a,ii), I(b,ii), I(c,ii), I(d,ii), and I(e,ii) shall mean a compound corresponding to Formula (I) having the substituents described in subparts (a) and (ii), subparts (b) and (ii), subparts (c) and (ii), subparts (d) and (ii), and subparts (e) and (ii), respectively, of Embodiment E.

A(a,ii), A(b,ii), A(c,ii), A(d,ii), and A(e,ii) shall mean a compound corresponding to Formula (I) having the substituents described in Embodiment A and subparts (a) and (ii), subparts (b) and (ii), subparts (c) and (ii), subparts (d) and (ii), and subparts (e) and (ii), respectively, of Embodiment E.

B(a,ii), B(b,ii), B(c,ii), B(d,ii), and B(e,ii) shall mean a compound corresponding to Formula (I) having the substituents described in Embodiment B and subparts (a) and (ii), subparts (b) and (ii), subparts (c) and (ii), subparts (d) and (ii), and subparts (e) and (ii), respectively, of Embodiment E.

C(a,ii), C(b,ii), C(c,ii), C(d,ii), and C(e,ii) shall mean a compound corresponding to Formula (I) having the substituents described in Embodiment C and subparts (a) and (ii), subparts (b) and (ii), subparts (c) and (ii), subparts (d) and (ii), and subparts (e) and (ii), respectively, of Embodiment E.

D(a,ii), D(b,ii), D(c,ii), D(d,ii), and D(e,ii) shall mean a compound corresponding to Formula (I) having the substituents described in Embodiment D and subparts (a) and (ii), subparts (b) and (ii), subparts (c) and (ii), subparts (d) and (ii), and subparts (e) and (ii), respectively, of Embodiment E.

I(a,c,ii), I(a,d,ii), I(a,e,ii), I(b,c,ii), I(b,d,ii), and I(b,e,ii) shall mean a compound corresponding to Formula (I) having the substituents described in subparts (a), (c) and (ii), subparts (a), (d) and (ii), subparts (a), (e) and (ii), subparts (b), (c) and (ii), subparts (b), (d) and (ii) and subparts (b), (e) and (ii), respectively, of Embodiment E.

A(a,c,ii), A(a,d,ii), A(a,e,ii), A(b,c,ii), A(b,d,ii), and A(b,e,ii) shall mean a compound corresponding to Formula (I) having the substituents described in Embodiment A and subparts (a), (c) and (ii), subparts (a), (d) and (ii), subparts (a), (e) and (ii), subparts (b), (c) and (ii), subparts (b), (d) and (ii) and subparts (b), (e) and (ii), respectively, of Embodiment E.

B(a,c,ii), B(a,d,ii), B(a,e,ii), B(b,c,ii), B(b,d,ii), and B(b,e,ii) shall mean a compound corresponding to Formula (I) having the substituents described in Embodiment B and subparts (a), (c) and (ii), subparts (a), (d) and (ii), subparts (a), (e) and (ii), subparts (b), (c) and (ii), subparts (b), (d) and (ii) and subparts (b), (e) and (ii), respectively, of Embodiment E.

C(a,c,ii), C(a,d,ii), C(a,e,ii), C(b,c,ii), C(b,d,ii), and C(b,e,ii) shall mean a compound corresponding to Formula (I) having the substituents described in Embodiment C and subparts (a), (c) and (ii), subparts (a), (d) and (ii), subparts (a), (e) and (ii), subparts (b), (c) and (ii), subparts (b), (d) and (ii) and subparts (b), (e) and (ii), respectively, of Embodiment E.

D(a,c,ii), D(a,d,ii), D(a,e,ii), D(b,c,ii), and D(b,d,ii) and D(b,e,ii) shall mean a compound corresponding to Formula (I) having the substituents described in Embodiment D and subparts (a), (c) and (ii), subparts (a), (d) and (ii), subparts (a), (e) and (ii), subparts (b), (c) and (ii), subparts (b), (d) and (ii) and subparts (b), (e) and (ii), respectively, of Embodiment E.

The abbreviations appearing in the Embodiment I of the embodiments section shall have the following meanings:

I[a] and I[b] shall mean a compound corresponding to Formula (I) having the substituents described in subparts (a) and (b), respectively, of Embodiment I.

A[a] and A[b] shall mean a compound corresponding to Formula (I) having the substituents described in Embodiment A and subparts (a) and (b), respectively, of Embodiment I.

B[a] and B[b] shall mean a compound corresponding to Formula (I) having the substituents described in Embodiment B and subparts (a) and (b), respectively, of Embodiment I.

C[a] and C[b] shall mean a compound corresponding to Formula (I) having the substituents described in Embodiment C and subparts (a) and (b), respectively, of Embodiment I.

D[a] and D[b] shall mean a compound corresponding to Formula (I) having the substituents described in Embodiment D and subparts (a) and (b), respectively, of Embodiment I.

I[a,i], I[a,ia], I[a,ii], I[a,iii], and I[a,iv] shall mean a compound corresponding to Formula (I) having the substituents described in subparts (a) and (i), subparts (a) and (ia), subparts (a) and (ii), subparts (a) and (iii), and subparts (a) and (iv), respectively, of Embodiment I.

A[a,i], A[a,ia], A[a,ii], I[a,iii], and I[a,iv] shall mean a compound corresponding to Formula (I) having the substituents described in Embodiment A and subparts (a) and (i), subparts (a) and (ia), subparts (a) and (ii), subparts (a) and (iii), and subparts (a) and (iv), respectively, of Embodiment I.

B[a,i], B[a,ia], B[a,ii], B[a,iii], and B[a,iv] shall mean a compound corresponding to Formula (I) having the substituents described in Embodiment B and subparts (a) and (i), subparts (a) and (ia), subparts (a) and (ii), subparts (a) and (iii), and subparts (a) and (iv), respectively, of Embodiment I.

C[a,i], C[a,ia], C[a,ii], C[a,iii], and C[a,iv] shall mean a compound corresponding to Formula (I) having the substituents described in Embodiment C and subparts (a) and (i), subparts (a) and (ia), subparts (a) and (ii), subparts (a) and (iii), and subparts (a) and (iv), respectively, of Embodiment I.

D[a,i], D[a,ia], D[a,ii], D[a,iii], and D[a,iv] shall mean a compound corresponding to Formula (I) having the substituents described in Embodiment D and subparts (a) and (i), subparts (a) and (ia), subparts (a) and (ii), subparts (a) and (iii), and subparts (a) and (iv), respectively, of Embodiment I.

I[b,i], I[b,ia], I[b,ii], I[b,iii], and I[b,iv] shall mean a compound corresponding to Formula (I) having the substituents described in subparts (b) and (i), subparts (a) and (ia), subparts (a) and (ii), subparts (a) and (iii), and subparts (a) and (iv), respectively, of Embodiment I.

A[b,i], A[b,ia], A[b,ii], I[b,iii], and I[b,iv] shall mean a compound corresponding to Formula (I) having the substituents described in Embodiment A and subparts (b) and (i), subparts (b) and (ia), subparts (b) and (ii), subparts (b) and (iii), and subparts (b) and (iv), respectively, of Embodiment I.

B[b,i], B[b,ia], B[b,ii], B[b,iii], and B[b,iv] shall mean a compound corresponding to Formula (I) having the substituents described in Embodiment B and subparts (b) and (i), subparts (b) and (ia), subparts (b) and (ii), subparts (b) and (iii), and subparts (b) and (iv), respectively, of Embodiment I.

C[b,i], C[b,ia], C[b,ii], C[b,iii], and C[b,iv] shall mean a compound corresponding to Formula (I) having the substituents described in Embodiment C and subparts (b) and (i), subparts (b) and (ia), subparts (b) and (ii), subparts (b) and (iii), and subparts (b) and (iv), respectively, of Embodiment I.

D[b,i], D[b,ia], D[b,ii], D[b,iii], and D[b,iv] shall mean a compound corresponding to Formula (I) having the substituents described in Embodiment D and subparts (b) and (i), subparts (b) and (ia), subparts (b) and (ii), subparts (b) and (iii), and subparts (b) and (iv), respectively, of Embodiment I.

Representative compounds of Formula (I) are as follows:
2-(4-(3-amino-4-(4-phenoxyphenyl)-1H-pyrazolo[4,3-c]pyridin-6-yl)piperidine-1-carbonyl)-3-cyclopropylacrylonitrile;
(R)-2-(4-(3-amino-4-(4-phenoxyphenyl)-1H-pyrazolo[4,3-c]pyridin-6-yl)piperidine-1-carbonyl)-3-cyclopropylacrylonitrile;
(S)-2-(4-(3-amino-4-(4-phenoxyphenyl)-1H-pyrazolo[4,3-c]pyridin-6-yl)piperidine-1-carbonyl)-3-cyclopropylacrylonitrile;
2-(3-(3-amino-4-(4-phenoxyphenyl)-1H-pyrazolo[4,3-c]pyridin-6-yl)piperidine-1-carbonyl)-3-cyclopropylacrylonitrile;
(R)-2-(3-(3-amino-4-(4-phenoxyphenyl)-1H-pyrazolo[4,3-c]pyridin-6-yl)piperidine-1-carbonyl)-3-cyclopropylacrylonitrile;
(S)-2-(3-(3-amino-4-(4-phenoxyphenyl)-1H-pyrazolo[4,3-c]pyridin-6-yl)piperidine-1-carbonyl)-3-cyclopropylacrylonitrile;
2-(2-((3-amino-4-(4-phenoxyphenyl)-1H-pyrazolo[4,3-c]pyridin-6-yl)methyl)-pyrrolidine-1-carbonyl)-3-cyclopropylacrylonitrile;
(R)-2-(2-((3-amino-4-(4-phenoxyphenyl)-1H-pyrazolo[4,3-c]pyridin-6-yl)methyl)-pyrrolidine-1-carbonyl)-3-cyclopropylacrylonitrile;
(S)-2-(2-((3-amino-4-(4-phenoxyphenyl)-1H-pyrazolo[4,3-c]pyridin-6-yl)methyl)-pyrrolidine-1-carbonyl)-3-cyclopropylacrylonitrile;
(R)-2-(2-((3-amino-4-(4-phenoxyphenyl)-1H-pyrazolo[4,3-c]pyridin-6-yl)methyl)-pyrrolidine-1-carbonyl)-4-(dimethylamino)-4-methylpent-2-enenitrile; MS (pos. ion) m/z: 550 (M+1);
(R)-2-(2-((3-amino-4-(4-phenoxyphenyl)-1H-pyrazolo[4,3-c]pyridin-6-yl)methyl)-pyrrolidine-1-carbonyl)-4-methylpent-2-enenitrile;
(R)-2-(2-((3-amino-4-(4-phenoxyphenyl)-1H-pyrazolo[4,3-c]pyridin-6-yl)methyl)-pyrrolidine-1-carbonyl)-4-methyl-4-(methylamino)pent-2-enenitrile;
(R)-4-amino-2-(2-((3-amino-4-(4-phenoxyphenyl)-1H-pyrazolo[4,3-c]pyridin-6-yl)-methyl)pyrrolidine-1-carbonyl)-4-methylpent-2-enenitrile;
(R)-2-(2-((3-amino-4-(4-phenoxyphenyl)-1H-pyrazolo[4,3-c]pyridin-6-yl)methyl)-pyrrolidine-1-carbonyl)-4-ethoxy-4-methylpent-2-enenitrile; MS (pos. ion) m/z: 551 (M+1);
(S)-2-(2-((3-amino-4-(4-phenoxyphenyl)-1H-pyrazolo[4,3-c]pyridin-6-yl)methyl)-pyrrolidine-1-carbonyl)-4-(dimethylamino)-4-methylpent-2-enenitrile; MS (pos. ion) m/z: 550 (M+1);
(S)-2-(2-((3-amino-4-(4-phenoxyphenyl)-1H-pyrazolo[4,3-c]pyridin-6-yl)methyl)-pyrrolidine-1-carbonyl)-4-methylpent-2-enenitrile;
(S)-2-(2-((3-amino-4-(4-phenoxyphenyl)-1H-pyrazolo[4,3-c]pyridin-6-yl)methyl)-pyrrolidine-1-carbonyl)-4-methyl-4-(methylamino)pent-2-enenitrile;
(S)-4-amino-2-(2-((3-amino-4-(4-phenoxyphenyl)-1H-pyrazolo[4,3-c]pyridin-6-yl)-methyl)pyrrolidine-1-carbonyl)-4-methylpent-2-enenitrile;
(S)-2-(2-((3-amino-4-(4-phenoxyphenyl)-1H-pyrazolo[4,3-c]pyridin-6-yl)methyl)-pyrrolidine-1-carbonyl)-4-ethoxy-4-methylpent-2-enenitrile; MS (pos. ion) m/z: 551 (M+1);
(R)-2-(3-(3-amino-4-(4-phenoxyphenyl)-1H-pyrazolo[4,3-c]pyridin-6-yl)piperidine-1-carbonyl)-4-methylpent-2-enenitrile;
(S)-2-(3-(3-amino-4-(4-phenoxyphenyl)-1H-pyrazolo[4,3-c]pyridin-6-yl)piperidine-1-carbonyl)-4-methylpent-2-enenitrile;
(R)-2-(3-(3-amino-4-(4-phenoxyphenyl)-1H-pyrazolo[4,3-c]pyridin-6-yl)piperidine-1-carbonyl)-4-(dimethylamino)-4-methylpent-2-enenitrile; MS (pos. ion) m/z: 550 (M+1);
(S)-2-(3-(3-amino-4-(4-phenoxyphenyl)-1H-pyrazolo[4,3-c]pyridin-6-yl)piperidine-1-carbonyl)-4-(dimethylamino)-4-methylpent-2-enenitrile; MS (pos. ion) m/z: 550 (M+1);
(S)-2-(3-(3-amino-4-(4-phenoxyphenyl)-1H-pyrazolo[4,3-c]pyridin-6-yl)piperidine-1-carbonyl)-4-methylpent-2-enenitrile; or
(R)-2-(3-(3-amino-4-(4-phenoxyphenyl)-1H-pyrazolo[4,3-c]pyridin-6-yl)piperidine-1-carbonyl)-4-methylpent-2-enenitrile;

or an (E) or (Z) isomer; or

a pharmaceutically acceptable salt thereof.

### EMBODIMENTS

### Embodiment A

In one embodiment, a compound of Formula (I) is as defined above (or a pharmaceutically acceptable salt thereof) in which the fused bicyclic moiety thereof has the structure:

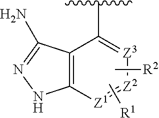

is:

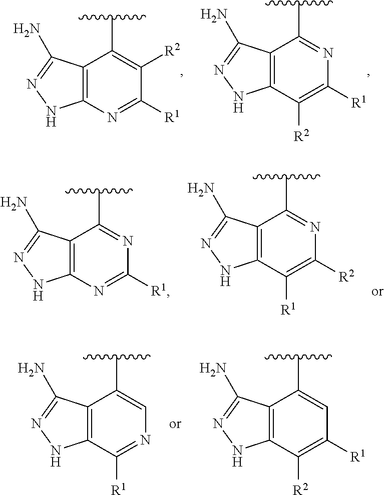

### Preferably,

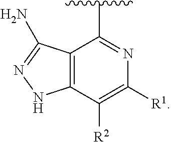

### Preferably,

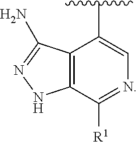

### Preferably,

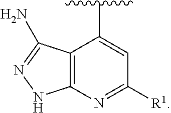

### Preferably,

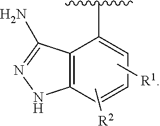

### Preferably,

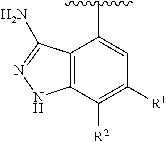

### Embodiment B

In another embodiment, a compound of Formula (I) is as defined above (or a pharmaceutically acceptable salt thereof) or as more specifically defined in embodiment (A) and groups contained therein, wherein in one group of compounds L is O, S, SO, SO₂, NR, NRCO, CONR, or NHCONH; preferably O, S, NH, or N(methyl), or NHCONH; more preferably L is O or NHCONH. Within this embodiment, in one group of compounds L is O. Within this embodiment, in one group of compounds L is NHCONH, NHCO, or CONH, preferably NHCONH. Within this embodiment and groups contained therein, in one group of compounds R² is hydrogen, methyl, fluoro, or trifluoromethyl, preferably hydrogen.

### Embodiment C

In another embodiment, a compound of Formula (I) is as defined above (or a pharmaceutically acceptable salt thereof) or as more specifically defined in embodiments (A) and/or (B) and groups contained therein, wherein in one group of compounds R³ and R⁴ are independently hydrogen, alkyl, alkoxy, cyano, halo, haloalkyl or haloalkoxy; preferably R³ and R⁴ are independently hydrogen, methyl, fluoro, methoxy, chloro, trifluoromethyl, or trifluoromethoxy. Preferably, R³ and R⁴ are independently hydrogen or fluoro. Preferably, in one group of compounds

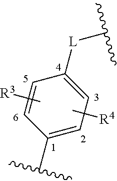

is a ring of formula:

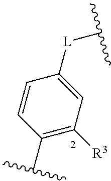

where R³ is hydrogen, methyl, ethyl, chloro, fluoro or trifluoromethyl, preferably methyl, ethyl, chloro or fluoro, more preferably, hydrogen, methyl, fluoro, or chloro, even more preferably hydrogen, chloro or fluoro, particularly preferably hydro en or fluoro. Preferably, in another group of compounds

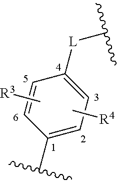

is a ring of formula

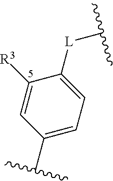

where R³ is alkyl or halo, preferably methyl, chloro or fluoro. Preferably, in yet another group of compounds

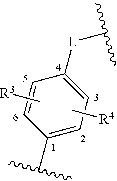

is a ring of formula

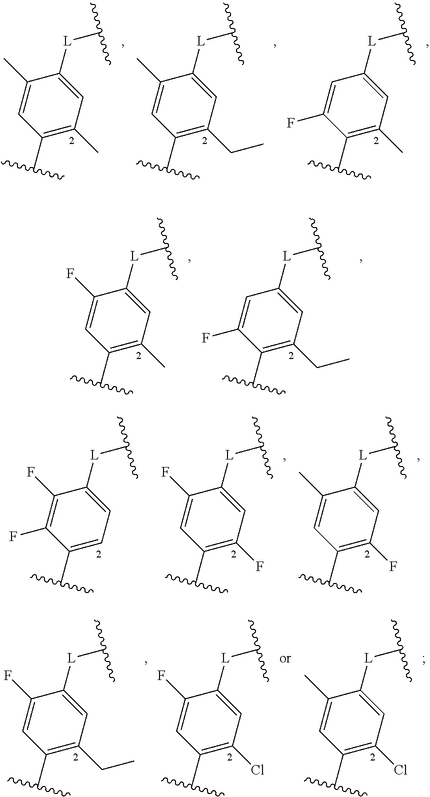

preferably,

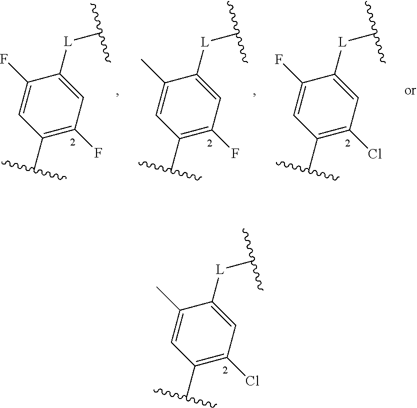

### Embodiment D

In another embodiment, within the compound of Formula (I) as defined above (or a pharmaceutically acceptable salt thereof) or as more specifically defined in embodiments (A), (B) and/or (C) and groups contained therein, in one group of compounds R⁶ and R⁷ are independently hydrogen, alkyl, alkoxy, halo, haloalkyl, haloalkoxy, or cyano. Preferably, R⁶ and R⁷ are independently hydrogen, methyl, methoxy, fluoro, chloro, trifluoromethyl, trifluoromethoxy, or cyano, more preferably R⁶ and R⁷ are hydrogen or fluoro.

### Embodiment E

In another embodiment, within the compound of Formula (I) as defined above and embodiments (A), (B), (C) and (D) and groups contained therein, in one group of compounds:

(a) R⁵ is hydrogen, alkyl, alkoxy, halo, haloalkyl, haloalkoxy, or cyano. Preferably, R⁵ is hydrogen, methyl, methoxy, fluoro, chloro, trifluoromethyl, trifluoromethoxy, or cyano; and

R¹ is —P-Q-CH═C(R^{b})(EWG) where P is a bond, NR^{a} (where R^{a} is hydrogen or alkyl), —O—, S, SO, SO₂, or alkylene, Q is a bond, aryl or heteroaryl, R^{b} is cyano, nitro, halo, haloalkyl, haloalkoxy, alkylthio, or alkylsulfonyl, EWG is an electron withdrawing group; and L is O.

(b) In another group of compounds R¹ is hydrogen, alkyl, alkoxy, halo, haloalkyl, haloalkoxy, or cyano. Preferably, R¹ is hydrogen, methyl, methoxy, fluoro, chloro, trifluoromethyl, trifluoromethoxy, or cyano; and

R⁵ is —P-Q-CH═C(R^{b})(EWG) where P is a bond, NR^{a} (where R^{a} is hydrogen or alkyl), —O—, S, SO, SO₂, alkylene or heteroalkylene, Q is a bond, aryl or heteroaryl, R^{b} is cyano, nitro, halo, haloalkyl, haloalkoxy, alkylthio, or alkylsulfonyl, EWG is an electron withdrawing group; and L is NHCONH, NHCO, or CONH, preferably NHCONH.

(c) Within groups in embodiment (E) e.g., I(a), I(b), A(a), A(b), B(a), B(b), C(a), C(b), D(a), and D(b), in one group of compounds —P— is bond, NR^{a}, O, or methylene and Q is aryl or heteroaryl, preferably, Q is selected from:

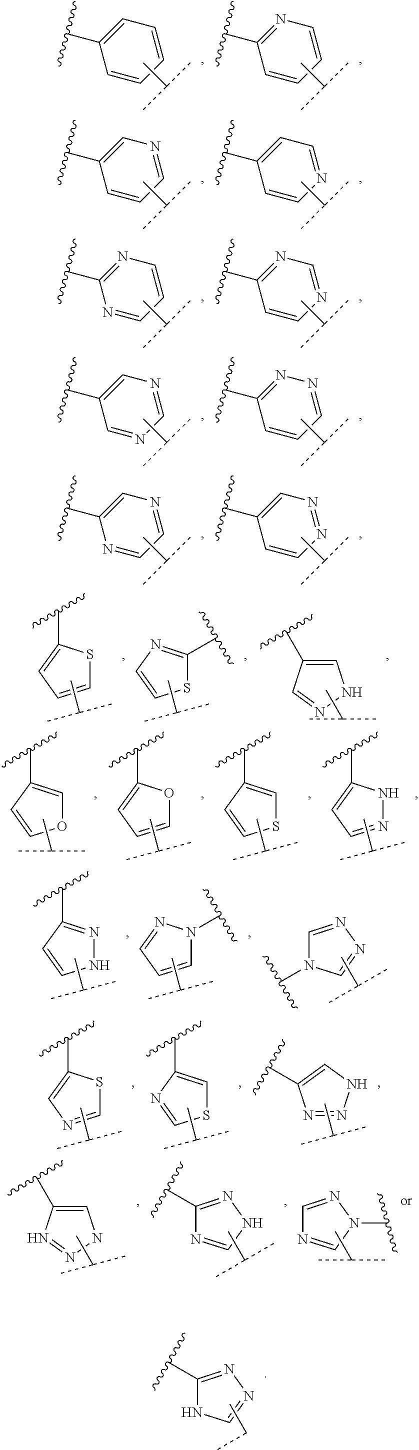

wherein each such ring is substituted with one, two or three substituents independently selected from hydrogen, alkyl, alkoxy, hydroxyl, cyano, nitro, halo, haloalkyl, haloalkoxy, alkylthio, alkylsulfonyl, carboxy, alkoxycarbonyl, aminocarbonyl or aminosulfonyl

wherein

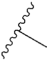

symbol denotes point of attachment of the ring to —P— when P is other than bond and directly to the rest of the molecule when P is a bond and

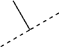

is bond attaching the ring to —CH═C(R^{b})(EWG).

(d) Within groups in embodiment (E), e.g., I(a), I(b), A(a), A(b), B(a), B(b), C(a), C(b), D(a), and D(b), in one group of compounds —P-Q- is selected from:

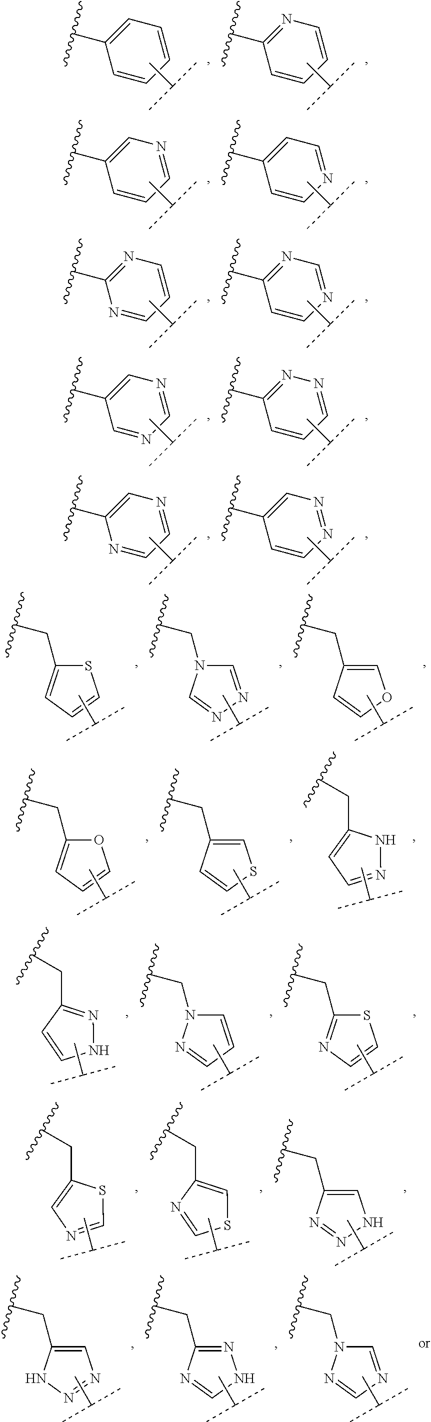

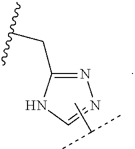

each substituted with one, two or three substituents independently selected from hydrogen, alkyl, alkoxy, hydroxyl, cyano, nitro, halo, haloalkyl, haloalkoxy, alkylthio, alkylsulfonyl, carboxy, alkoxycarbonyl, aminocarbonyl or aminosulfonyl.

(e) Within the groups in embodiment (E) i.e., I(a), I(b), A(a), A(b), B(a), B(b), C(a), C(b), D(a), and D(b) in another group of compounds —P-Q- is selected from: phenyl, 2-, 3-, or 4-pyridyl substituted as defined above or unsubstituted.

(f) Within the groups in embodiment I(b), A(b), B(b), C(b), and/or D(b) in embodiment E, in one group of compounds —P-Q is a bond.
(i) Within the above groups in embodiment (E), e.g., Formula I, I(a), I(b), I(c), I(d), I(e), A(a), A(b), A(c), A(d), A(e), B(a), B(b), B(c), B(d), B(e), C(a), C(b), C(c), C(d), C(e), D(a), D(b), D(c), D(d), D(e), I(a,c), I(a,d), I(a,e), I(b,c), I(b,d), I(b,e), A(a,c), A(a,d), A(a,e), A(b,c), A(b,d), A(b,e), B(a,c), B(a,d), B(a,e), B(b,c), B(b,d), B(b,e), C(a,c), C(a,d), C(a,e), C(b,c), C(b,d), C(b,e), D(a,c), D(a,d), D(a,e), D(b,c), D(b,d), and D(b,e), and groups contained therein, in one group of compounds when Q is a six membered ring, then the —CH═C(R^{b})(EWG) group is attached to the carbon atom in the six membered ring that is preferably meta to the carbon atom that attaches the six membered ring to —P—.
(ii) Within the above groups in embodiment (E), e.g., Formula I, I(a), I(b), I(c), I(d), I(e), A(a), A(b), A(c), A(d), A(e), B(a), B(b), B(c), B(d), B(e), C(a), C(b), C(c), C(d), C(e), D(a), D(b), D(c), D(d), D(e), I(a,c), I(a,d), I(a,e), I(b,c), I(b,d), I(b,e), A(a,c), A(a,d), A(a,e), A(b,c), A(b,d), A(b,e), B(a,c), B(a,d), B(a,e), B(b,c), B(b,d), B(b,e), C(a,c), C(a,d), C(a,e), C(b,c), C(b,d), C(b,e), D(a,c), D(a,d), D(a,e), D(b,c), D(b,d), and D(b,e), and groups contained therein, in one group of compounds when Q is a five membered ring, then the —CH═C(R^{b})(EWG) group is attached to the atom in the five membered ring that is preferably ortho to the atom that attaches the five membered ring to —P—.
(iii) Within the above groups in embodiment (E), e.g., Formula I, I(a), I(b), I(c), I(d), I(e), A(a), A(b), A(c), A(d), A(e), B(a), B(b), B(c), B(d), B(e), C(a), C(b), C(c), C(d), C(e), D(a), D(b), D(c), D(d), D(e), I(a,c), I(a,d), I(a,e), I(b,c), I(b,d), I(b,e), I(b,f), A(a,c), A(a,d), A(a,e), A(b,c), A(b,d), A(b,e), A(b,f), B(a,c), B(a,d), B(a,e), B(b,c), B(b,d), B(b,e), B(b,f), C(a,c), C(a,d), C(a,e), C(b,c), C(b,d), C(b,e), C(b,f), D(a,c), D(a,d), D(a,e), D(b,c), D(b,d), D(b,e), D(b,f), I(a,i), I(b,i), I(c,i), I(d,i), I(e,i), A(a,i), A(b,i), A(c,i), A(d,i), A(e,i), B(a,i), B(b,i), B(c,i), B(d,i), B(e,i), C(a,i), C(b,i), C(c,i), C(d,i), C(e,i), D(a,i), D(b,i), D(c,i), D(d,i), D(e,i), I(a,c,i), I(a,d,i), I(a,e,i), I(b,c,i), I(b,d,i), I(b,e,i), A(a,c,i), A(a,d,i), A(a,e,i), A(b,c,i), A(b,d,i), A(b,e,i), B(a,c,i), B(a,d,i), B(a,e,i), B(b,c,i), B(b,d,i), B(b,e,i), C(a,c,i), C(a,d,i), C(a,e,i), C(b,c,i), C(b,d,i), C(b,e,i), D(a,c,i), D(a,d,i), D(a,e,i), D(b,c,i), D(b,d,i), D(b,e,i), I(a,ii), I(b,ii), I(c,ii), I(d,ii), I(e,ii), A(a,ii), A(b,ii), A(c,ii), A(d,ii), A(e,ii), B(a,ii), B(b,ii), B(c,ii), B(d,ii), B(e,ii), C(a,ii), C(b,ii), C(c,ii), C(d,ii), C(e,ii), D(a,ii), D(b,ii), D(c,ii), D(d,ii), D(e,ii), I(a,c,ii), I(a,d,ii), I(a,e,ii), I(b,c,ii), I(b,d,ii), I(b,e,ii), A(a,c,ii), A(a,d,ii), A(a,e,ii), A(b,c,ii), A(b,d,ii), A(b,e,ii), B(a,c,ii), B(a,d,ii), B(a,e,ii), B(b,c,ii), B(b,d,ii), B(b,e,ii), C(a,c,ii), C(a,d,ii), C(a,e,ii), C(b,c,ii), C(b,d,ii), C(b,e,ii), D(a,c,ii), D(a,d,ii), D(a,e,ii), D(b,c,ii), D(b,d,ii) and D(b,e,ii), and groups contained therein, in one group of compounds R^{b} is cyano.
(iv) Within the above groups in embodiment (E), e.g., Formula I, I(a), I(b), I(c), I(d), I(e), A(a), A(b), A(c), A(d), A(e), B(a), B(b), B(c), B(d), B(e), C(a), C(b), C(c), C(d), C(e), D(a), D(b), D(c), D(d), D(e), I(a,c), I(a,d), I(a,e), I(b,c), I(b,d), I(b,e), I(b,f), A(a,c), A(a,d), A(a,e), A(b,c), A(b,d), A(b,e), B(a,c), B(a,d), B(a,e), B(b,c), B(b,d), B(b,e), B(b,f), C(a,c), C(a,d), C(a,e), C(b,c), C(b,d), C(b,e), C(b,f), D(a,c), D(a,d), D(a,e), D(b,c), D(b,d), D(b,e), D(b,f), I(a,i), I(b,i), I(c,i), I(d,i), I(e,i), A(a,i), A(b,i), A(c,i), A(d,i), A(e,i), B(a,i), B(b,i), B(c,i), B(d,i), B(e,i), C(a,i), C(b,i), C(c,i), C(d,i), C(e,i), D(a,i), D(b,i), D(c,i), D(d,i), D(e,i), I(a,c,i), I(a,d,i), I(a,e,i), I(b,c,i), I(b,d,i), I(b,e,i), A(a,c,i), A(a,d,i), A(a,e,i), A(b,c,i), A(b,d,i), A(b,e,i), B(a,c,i), B(a,d,i), B(a,e,i), B(b,c,i), B(b,d,i), B(b,e,i), C(a,c,i), C(a,d,i), C(a,e,i), C(b,c,i), C(b,d,i), C(b,e,i), D(a,c,i), D(a,d,i), D(a,e,i), D(b,c,i), D(b,d,i), D(b,e,i), I(a,ii), I(b,ii), I(c,ii), I(d,ii), I(e,ii), A(a,ii), A(b,ii), A(c,ii), A(d,ii), A(e,ii), B(a,ii), B(b,ii), B(c,ii), B(d,ii), B(e,ii), C(a,ii), C(b,ii), C(c,ii), C(d,ii), C(e,ii), D(a,ii), D(b,ii), D(c,ii), D(d,ii), D(e,ii), I(a,c,ii), I(a,d,ii), I(a,e,ii), I(b,c,ii), I(b,d,ii), I(b,e,ii), A(a,c,ii), A(a,d,ii), A(a,e,ii), A(b,c,ii), A(b,d,ii), A(b,e,ii), B(a,c,ii), B(a,d,ii), B(a,e,ii), B(b,c,ii), B(b,d,ii), B(b,e,ii), C(a,c,ii), C(a,d,ii), C(a,e,ii), C(b,c,ii), C(b,d,ii), C(b,e,ii), D(a,c,ii), D(a,d,ii), D(a,e,ii), D(b,c,ii), D(b,d,ii) and D(b,e,i), and groups contained therein, in another group of compounds R^{b} is trifluoromethyl.
(v) Within the above groups in embodiment (E), e.g., Formula I, I(a), I(b), I(c), I(d), I(e), A(a), A(b), A(c), A(d), A(e), B(a), B(b), B(c), B(d), B(e), C(a), C(b), C(c), C(d), C(e), D(a), D(b), D(c), D(d), D(e), I(a,c), I(a,d), I(a,e), I(b,c), I(b,d), I(b,e), A(a,c), A(a,d), A(a,e), A(b,c), A(b,d), A(b,e), B(a,c), B(a,d), B(a,e), B(b,c), B(b,d), B(b,e), C(a,c), C(a,d), C(a,e), C(b,c), C(b,d), C(b,e), D(a,c), D(a,d), D(a,e), D(b,c), D(b,d), D(b,e), I(a,i), I(b,i), I(c,i), I(d,i), I(e,i), A(a,i), A(b,i), A(c,i), A(c,i), A(d,i), A(e,i), B(a,i), B(b,i), B(c,i), B(d,i), B(e,i), C(a,i), C(b,i), C(c,i), C(d,i), C(e,i), D(a,i), D(b,i), D(c,i), D(d,i), D(e,i), I(a,c,i), I(a,d,i), I(a,e,i), I(b,c,i), I(b,d,i), I(b,e,i), A(a,c,i), A(a,d,i), A(a,e,i), A(b,c,i), A(b,d,i), A(b,e,i), B(a,c,i), B(a,d,i), B(a,e,i), B(b,c,i), B(b,d,i), B(b,e,i), C(a,c,i), C(a,d,i), C(a,e,i), C(b,c,i), C(b,d,i), C(b,e,i), D(a,c,i), D(a,d,i), D(a,e,i), D(b,c,i), D(b,d,i), D(b,e,i), I(a,ii) I(b,ii), I(c,ii), I(d,ii), I(e,ii), A(a,ii), A(b,ii), A(c,ii), A(d,ii), A(e,ii), B(a,ii), B(b,ii), B(c,ii), B(d,ii), B(e,ii), C(a,ii), C(b,ii), C(c,ii), C(d,ii), C(e,ii), D(a,ii), D(b,ii), D(c,ii), D(d,ii), D(e,ii), I(a,c,ii), I(a,d,ii), I(a,e,ii), I(b,c,ii), I(b,d,ii), I(b,e,ii), A(a,c,ii), A(a,d,ii), A(a,e,ii), A(b,c,ii), A(b,d,ii), A(b,e,ii), B(a,c,ii), B(a,d,ii), B(a,e,ii), B(b,c,ii), B(b,d,ii), B(b,e,ii), C(a,c,ii), C(a,d,ii), C(a,e,ii), C(b,c,ii), C(b,d,ii), C(b,e,ii), D(a,c,ii), D(a,d,ii), D(a,e,ii), D(b,c,ii), D(b,d,ii) and D(b,e,i), and groups contained therein, in another group of compounds R^{b} is nitro, methylthio or methylsulfonyl.

### Embodiment F

In another embodiment, within the compound of Formula (I) as defined above and embodiments (A), (B), (C), (D), and/or (E) and groups contained therein, in one group of compounds:

EWG is —N(R′₃)⁺, —SO₃H, —SO₃R′, —S(O₂)R′, —S(O)R′, —C(O)NH₂, —C(O)NHR^{g}, —C(O)NR^{f}R^{g}, —S(O₂)NH₂, —SO₂NHRⁱ′, —SO₂NR^{h}Rⁱ, —PO(OR′)₂, —PO₃H₂, —PO(NR′₂)₂, —C≡N, —CH(haloalkyl), —C(O)X′, —COOH, —COOR′, —C(O)R′, —C(O)H, —P(O)(OR′)OR″, halo, heteroaryl, or aryl wherein X′ is independently halogen (e.g. chloro of fluoro), and R′, R″, R^{f}, R^{g}, R^{h}, and Rⁱ are independently hydrogen, alkyl, substituted alkyl, cycloalkyl, cycloalkyleneNR^{d}R^{e} (where R^{d} and R^{e} are independently hydrogen, alkyl, or cycloalkyl) or R^{f} and R^{g} and R^{h} and Rⁱ together with the nitrogen atom to which they are attached form heterocycloamino; and heterocycloamino, aryl and heteroaryl are substituted with one, two or three substituents independently selected from hydrogen, alkyl, alkoxy, hydroxyl, cyano, nitro, halo, haloalkyl, haloalkoxy, alkylthio, alkylsulfonyl, carboxy, alkoxycarbonyl, aminocarbonyl or aminosulfonyl.

Preferably, EWG is —CO—NR^{f}R^{g} or —SO₂NR^{h}Rⁱ (wherein R^{f} and R^{h} are independently hydrogen, alkyl, or cycloalkyl and R^{g} and Rⁱ are independently hydrogen, alkyl, substituted alkyl, cycloalkyleneNR^{d}R^{e} (where R^{d} and R^{e} are independently hydrogen, alkyl, or cycloalkyl; or R^{d} and R^{e} together with the nitrogen atom to which they are attached form heterocycloamino), or R^{f} and R^{g} and R^{h} and Rⁱ together with the nitrogen atom to which they are attached form heterocycloamino) and aryl or heteroaryl wherein each of the aforementioned ring is substituted with one, two or three substituents independently selected from hydrogen, alkyl, alkoxy, hydroxyl, cyano, nitro, halo, haloalkyl, haloalkoxy, alkylthio, alkylsulfonyl, carboxy, alkoxycarbonyl, aminocarbonyl or aminosulfonyl. Preferably, the heteroaryl ring is pyridinyl, pyrazolyl, indazolyl, indolyl, thienyl, pyrrolyl, imidazolyl, thiazolyl, benzothiazolyl, oxazolyl, benzimidazolyl, benzoxazolyl, isoxazolyl, benzisoxazolyl, triazolyl, benzotriazolyl, quinolinyl, isoquinolinyl, quinazolinyl, or pyrimidinyl each substituted as defined above.

Within the groups in embodiment F, in one group of compounds EWG is pyridine-2-yl, pyridine-3-yl, pyridine-4-yl, pyrrol-1-yl, pyrazol-1-yl, or thiazol-2-yl.

Within the groups in embodiment F, in another group of compounds EWG is dimethylaminocarbonyl, methylaminocarbonyl, isopropylaminocarbonyl, tert-butylaminocarbonyl, or 3-hydroxy-1-methylpropylaminocarbonyl.

Within the groups in embodiment F, in yet another group of compounds EWG is azetidin-1-ylcarbonyl, 4-hydroxyazetidin-1-ylcarbonyl, pyrrolidin-1-ylcarbonyl, 4-ethylpiperazin-1-ylcarbonyl, or 2,6-dimethylmorpholine-4-ylcarbonyl.

Preferably, EWG is —CON(CH₃)₂, —CONHcyclopropyl, or

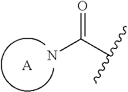

where ring A is heterocycloamino (such as piperazinyl or piperidinyl) optionally substituted with hydroxyl, methyl, methoxy, amino, methylamino or dimethylamino, preferably ring A is piperazinyl or piperidinyl substituted at the 3 or 4 position, the nitrogen atom attached to the carbonyl group being position 1.

Preferably, EWG is aryl or heteroaryl ring heteroaryl wherein each of the aforementioned ring is substituted with one, two or three substituents independently selected from hydrogen, alkyl, alkoxy, hydroxyl, cyano, nitro, halo, haloalkyl, haloalkoxy, alkylthio, alkylsulfonyl, carboxy, alkoxycarbonyl, aminocarbonyl or aminosulfonyl.

Preferably, EWG is selected from:

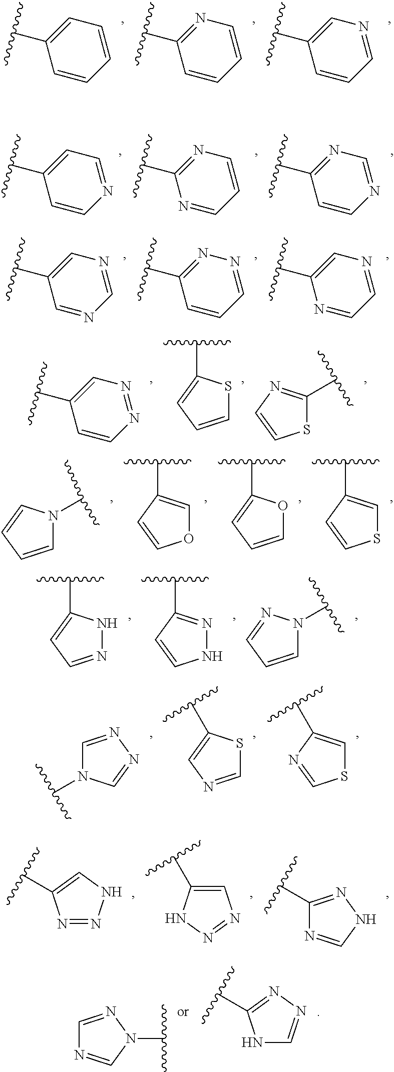

and is substituted with one, two or three substituents independently selected from hydrogen, halo, haloalkyl, cyano, haloalkoxy, or alkylsulfonyl, preferably hydrogen, fluoro, cyano, trifluoromethyl, trifluoromethoxy, or cyano.

### Embodiment G

In another embodiment, within the compound of Formula (I) as defined above and embodiments (A), (B), (C), (D), and/or (E) and groups contained therein, in one group of compounds:

EWG is:

(i) —C(O)NR^{f}R^{g} where R^{f} is hydrogen or alkyl and R^{g} is substituted alkyl, preferably R^{g} is (C₂-C₆)alkylene substituted with hydroxyl, alkoxy, alkylamino, dialkylamino or heterocycloamino or R^{f} and R^{g} together with the nitrogen atom to which they are attached form heterocycloamino. Preferably, R^{g} is 2-hydroxyethyl, 2-methoxyethyl, 2-methylaminoethyl, or 2-dimethylaminoethyl; or R^{f} and R^{g} together with the nitrogen atom to which they are attached form pyrrolidinyl, piperidinyl, piperazinyl, or morpholinyl ring wherein the pyrrolidinyl, piperidinyl, and piperazinyl rings are optionally substituted with hydroxyl, methyl, methoxy, amino, methylamino or dimethylamino, preferably at the 3 or 4 position of the pyrrolidinyl, piperidinyl, and piperazinyl rings; or

(ii) —COOR′ where R is hydrogen or alkyl, preferably methyl, ethyl, isopropyl, or tert-butyl; or

(iii) —SO₂alkyl, —SO₂NHalkyl, —SO₂N(alkyl)₂, or —S(O)₂NR^{h}Rⁱ where R^{h} and Rⁱ together with the nitrogen atom to which they are attached form heterocycloamino. Preferably EWG is methylsulfonyl, methylaminosulfonyl, dimethylaminosulfonyl or R^{h} and Rⁱ together with the nitrogen atom to which they are attached form pyrrolidinyl, piperidinyl, piperazinyl, or morpholinyl ring wherein the pyrrolidinyl, piperidinyl, and piperazinyl rings are optionally substituted with hydroxyl, methyl, methoxy, amino, methylamino or dimethylamino, preferably the substituent is at the 3 or 4 position of the pyrrolidinyl, piperidinyl, and piperazinyl rings; or

(iv) a 5 or six membered heteroaryl ring is substituted with one, two or three substituents independently selected from hydrogen, alkyl, alkoxy, hydroxyl, cyano, nitro, halo, haloalkyl, haloalkoxy, alkylthio, alkylsulfonyl, carboxy, alkoxycarbonyl, aminocarbonyl or aminosulfonyl. Preferably, the heteroaryl ring is selected from:

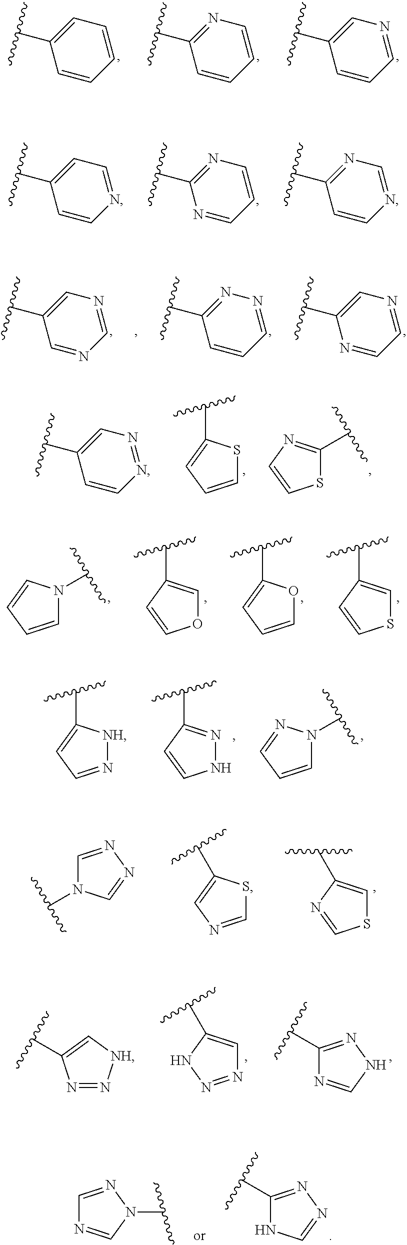

and is substituted with one, two or three substituents independently selected from hydrogen, halo, haloalkyl, cyano, haloalkoxy, or alkylsulfonyl, preferably hydrogen, fluoro, cyano, trifluoromethyl, trifluoromethoxy, or cyano.

(v) Preferably, EWG is —C(O)NR^{f}R^{g} where R^{f} is hydrogen or alkyl and R^{g} is substituted alkyl, preferably R^{g} is (C₂-C₆)alkylene substituted with hydroxyl, alkoxy, alkylamino, dialkylamino or heterocycloamino or R^{f} and R^{g} together with the nitrogen atom to which they are attached form heterocycloamino. Preferably, R^{g} is 2-hydroxyethyl, 2-methoxyethyl, 2-methylaminoethyl, or 2-dimethylaminoethyl; or R^{f} and R^{g} together with the nitrogen atom to which they are attached form pyrrolidinyl, piperidinyl, piperazinyl, or morpholinyl ring wherein the pyrrolidinyl, piperidinyl, and piperazinyl rings are optionally substituted with hydroxyl, methyl, methoxy, amino, methylamino or dimethylamino, preferably at the 3 or 4 position of the pyrrolidinyl, piperidinyl, and piperazinyl rings.

(vi) Preferably, EWG is a 5 or six membered heteroaryl ring is substituted with one, two or three substituents independently selected from hydrogen, alkyl, alkoxy, hydroxyl, cyano, nitro, halo, haloalkyl, haloalkoxy, alkylthio, alkylsulfonyl, carboxy, alkoxycarbonyl, aminocarbonyl or aminosulfonyl. Preferably, the heteroaryl ring is selected from:

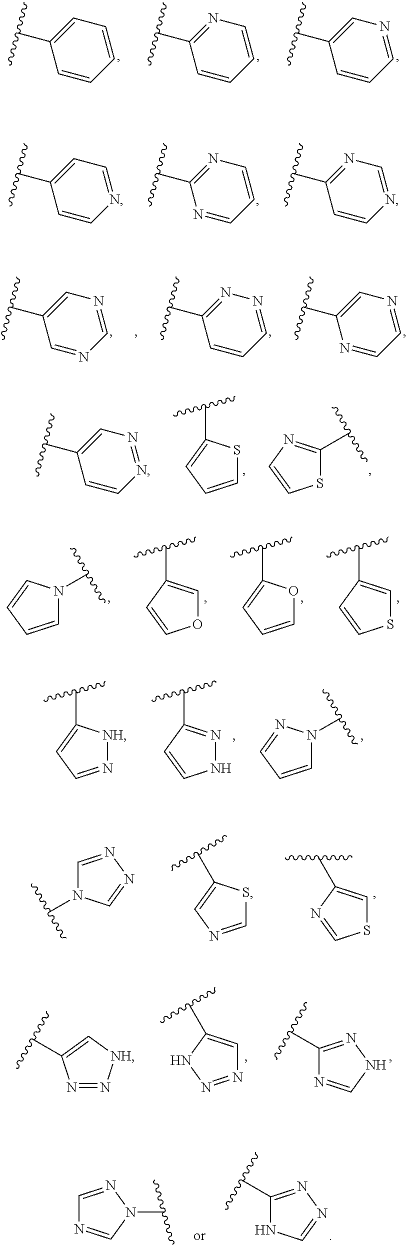

and is substituted with one, two or three substituents independently selected from hydrogen, halo, haloalkyl, cyano, haloalkoxy, or alkylsulfonyl, preferably hydrogen, fluoro, cyano, trifluoromethyl, trifluoromethoxy, or cyano.

(vii) Preferably, EWG is —COOR′ where R is hydrogen or alkyl, preferably methyl, ethyl, isopropyl, or tert-butyl; or —SO₂alkyl, —SO₂NHalkyl, —SO₂N(alkyl)₂, or —S(O)₂NR^{h}Rⁱ where R^{h} and Rⁱ together with the nitrogen atom to which they are attached form heterocycloamino.

(viii) Preferably, EWG is methylsulfonyl, methylaminosulfonyl, dimethylaminosulfonyl or —S(O)₂NR^{h}Rⁱ where R^{h} and Rⁱ together with the nitrogen atom to which they are attached form pyrrolidinyl, piperidinyl, piperazinyl, or morpholinyl ring wherein the pyrrolidinyl, piperidinyl, and piperazinyl rings are optionally substituted with hydroxyl, methyl, methoxy, amino, methylamino or dimethylamino, preferably the substituent is at the 3 or 4 position of the pyrrolidinyl, piperidinyl, and piperazinyl rings,

Within embodiment (G) and groups contained therein i.e, (i)-(vii), in one group of compounds —P-Q- are as described in embodiment (E) above, and groups contained therein i.e, (c), (d), (i) and (ii) above or combinations thereof.

### Embodiment H

In another embodiment, within the compound of Formula (I) as defined above and embodiments (A), (B), (C), and/or (D), and groups contained therein, in one group of compounds:

(a) R⁵ is hydrogen, alkyl, alkoxy, halo, haloalkyl, haloalkoxy, or cyano. Preferably, R⁵ is hydrogen, methyl, methoxy, fluoro, chloro, trifluoromethyl, trifluoromethoxy, or cyano; R¹ a group of formula (a)

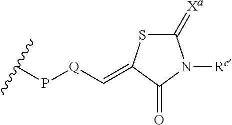

where X^{a} is O, S or N(H or alkyl) and R^{c′} is hydrogen, alkyl, cycloalkyl, substituted alkyl or cycloalkyleneNR^{d}R^{e} and L is O. Preferably, X^{a} is O, and R^{c′} is hydrogen, methyl, ethyl, propyl, cyclopropyl, or alkylene substituted with hydroxyl, alkoxy, alkylamino or dialkylamino. Preferably, X^{a} is O, and R^{c′} is hydrogen, methyl, ethyl, propyl, cyclopropyl, 2-hydroxyethyl, 2-methoxyethyl, 2-methylaminoethyl or 2-dimethylaminoethyl.

(b) In another group of compounds R¹ is hydrogen, alkyl, alkoxy, halo, haloalkyl, haloalkoxy, or cyano. Preferably, R¹ is hydrogen, methyl, methoxy, fluoro, chloro, trifluoromethyl, trifluoromethoxy, or cyano; and

R⁵ a group of formula (a)

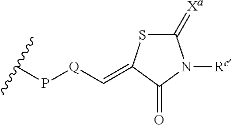

where X^{a} is O, S or N(H or alkyl) and R^{c′} is hydrogen, alkyl, cycloalkyl, substituted alkylalkyl or cycloalkyleneNR^{d}R; and L is NHCONH, NHCO, or CONH. Preferably, X^{a} is O, and R^{c′} is hydrogen, methyl, ethyl, propyl, cyclopropyl, or alkylene substituted with hydroxyl, alkoxy, alkylamino or dialkylamino. Preferably, X^{a} is O, and R^{c′} is hydrogen, methyl, ethyl, propyl, cyclopropyl, 2-hydroxyethyl, 2-methoxyethyl, 2-methylaminoethyl or 2-dimethylaminoethyl.

Within embodiment (H) and groups contained therein, in one group of compounds —P-Q- are as described in embodiment (E) above, and groups contained therein, i.e, (c), (d), (e), (f), (i) and (ii) above or combinations thereof. Preferably, —P-Q- are together alkylene, heteroalkylene, aryl or heteroaryl, more preferably phenyl or heteroaryl substituted as defined above.

Within embodiment (H) and groups contained therein, in one group of compounds —P-Q- is a bond.

Within embodiment (H) and groups contained therein, in another group of compounds P is alkylene, preferably methylene and Q is a bond.

### Embodiment I

In another embodiment, within the compound of Formula (I) as defined above and embodiments (A), (B), (C) and/or (D) and groups contained therein:

(a) in one group of compounds:

R¹ is hydrogen, alkyl, hydroxy, alkoxy, halo, haloalkyl, or haloalkoxy and R⁵ is —Z-(EWG′)-C(R^{b})═CHR^{c} where Z is bond, NR^{a} (where R^{a} is hydrogen or alkyl), —O—, S, SO, SO₂, alkylene, phenyl, heteroaryl, or heteroalkylene, EWG′ is an electron withdrawing group, R^{b} is cyano, nitro, halo, haloalkyl, haloalkoxy, alkylthio, or alkylsulfonyl and R^{c} is alkyl, substituted alkyl, cycloalkyl, cycloalkyleneNR^{d}R^{e} where R^{d} and R^{e} are independently hydrogen, alkyl, or cycloalkyl; and L is NHCONH, NHCO, or CONH.

(b) In another group of compounds:

R⁵ is hydrogen, alkyl, hydroxy, alkoxy, halo, haloalkyl, or haloalkoxy and R¹ is —Z-(EWG′)-C(R^{b})═CHR where Z is bond, NR^{a} (where R^{a} is hydrogen or alkyl), —O—, S, SO, SO₂, alkylene, phenyl, heteroaryl, or heteroalkylene, EWG′ is an electron withdrawing group, R^{b} is cyano, nitro, halo, haloalkyl, haloalkoxy, alkylthio, or alkylsulfonyl and R^{c} is alkyl, substituted alkyl, cycloalkyl, cycloalkyleneNR^{d}R^{e} where R^{d} and R^{e} are independently hydrogen, alkyl, or cycloalkyl and L is O.

(i) With groups (a) and (b), in one group of compounds Z is bond, cycloalkylene, phenyl, heteroaryl, or alkylene and EWG′ is —NR′CO— or —NR′SO₂; wherein each R′ is independently hydrogen or alkyl; R^{b} is cyano, nitro, fluoro, trifluoromethyl, 2,2,2-trifluoroethyl, trifluoromethoxy, 2,2,2-trifluoroethyloxy, methylsulfonyl or methylthio and R^{c} is methyl, isopropyl, tert-butyl, cyclopropyl, trifluoromethyl, 2,2,2-trifluoroethyl, 1-methyl-1-methylaminoethyl, 1-methyl-1-dimethylaminoethyl, 1-methyl-1-aminoethyl, 1-methylaminocycloprop-1-ylene, or 1-dimethylaminocycloprop-1-ylene. Within groups in (i) in one group of compounds EWG′ is —NHCO— and Z is

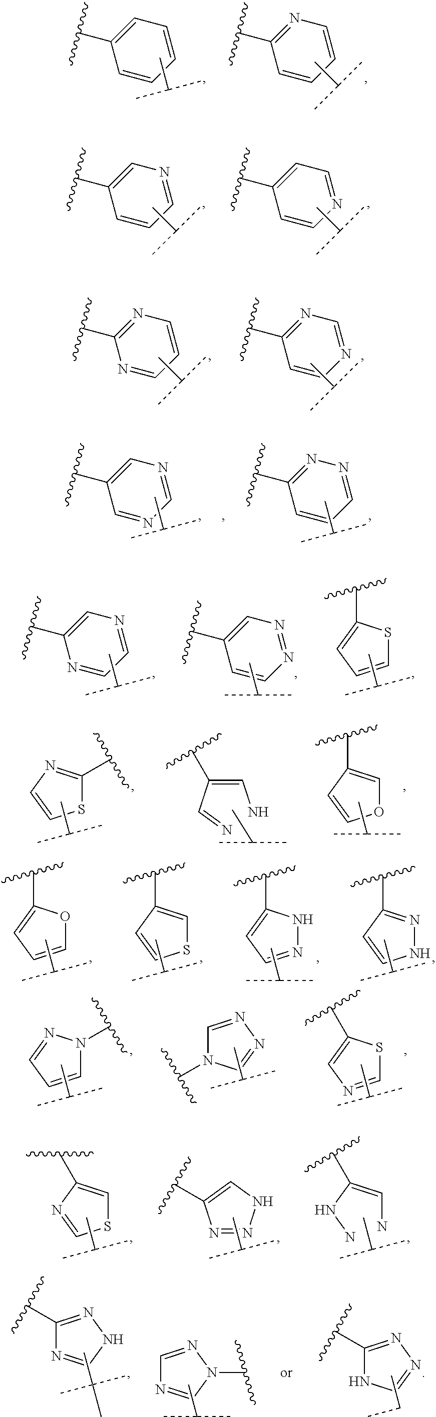

preferably phenyl. Preferably, EWG′ is at the meta position of the phenyl ring (meta to bond attaching Z to core).

(ia) Preferably R^{s} is —C(R^{b})═CHR^{c} in (I)(a) when Ar is electron withdrawing eg pyridinyl and —C(R^{b})═CHR is attached at carbon ortho to the N atom on the pyridinyl ring.

(ii) With groups (a) and (b), in one group of compounds Z is bond, NR^{a}, O or alkylene, preferably methylene, and EWG′ is

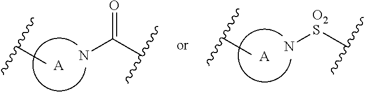

where ring A is heterocycloamino, preferably A is piperidinyl or pyrrolidinyl, preferably —Z-EWG- is 3-piperidin-1-ylcarbonyl or 2-CH₂-pyrrolidin-1-ylcarbonyl, and R^{b} is cyano, nitro, fluoro, trifluoromethyl, 2,2,2-trifluoroethyl, trifluoromethoxy, 2,2,2-trifluoroethyloxy, methylsulfonyl or methylthio, preferably cyano, and R^{c} methyl, isopropyl, tert-butyl, cyclopropyl, trifluoromethyl, 2,2,2-trifluoroethyl, 1-methyl-1-methylaminoethyl, 1-methyl-1-dimethylaminoethyl, 1-methyl-1-aminoethyl, 1-methylaminocycloprop-1-ylene, or 1-dimethylaminocycloprop-1-ylene. Preferably —Z-EWG′- is 3-piperidin-1-ylcarbonyl or —CH₂-2-pyrrolidin-1-ylcarbonyl (i.e., methylene is attached at 2-position of pyrrolidin-1-ylcarbonyl ring). Preferably, —Z-EWG′- is 3-piperidin-1-ylcarbonyl or 2-CH₂pyrrolidin-1-ylcarbonyl (i.e., methylene is attached at 2-position of pyrrolidin-1-ylcarbonyl ring) and R^{c} is isopropyl, tert-butyl, cyclopropyl, 1-methyl-1-methylaminoethyl, 1-methyl-1-dimethylaminoethyl, or 1-methyl-1-aminoethyl, more preferably R^{c} is isopropyl. Preferably, —Z-EWG′- is 3-piperidin-1-ylcarbonyl or 2-CH₂pyrrolidin-1-ylcarbonyl (i.e., methylene is attached at 2-position of pyrrolidin-1-ylcarbonyl ring) and R^{c} is tert-butyl. Preferably, —Z-EWG′- is 3-piperidin-1-ylcarbonyl or 2-CH₂pyrrolidin-1-ylcarbonyl (i.e., methylene is attached at 2-position of pyrrolidin-1-ylcarbonyl ring) and R^{c} is cyclopropyl. Preferably, —Z-EWG′- is 3-piperidin-1-ylcarbonyl or 2-CH₂pyrrolidin-1-ylcarbonyl (i.e., methylene is attached at 2-position of pyrrolidin-1-ylcarbonyl ring) and R^{c} is 1-methyl-1-methylaminoethyl, 1-methyl-1-dimethylaminoethyl, or 1-methyl-1-aminoethyl, preferably 1-methyl-1-dimethylaminoethyl. (iii) Within groups (a) and (b), in one group of compounds Z is bond, NR^{a}, O or alkylene and EWG′ is heteroaryl, or aryl; wherein R^{a} is independently hydrogen or alkyl; and aryl and heteroaryl are substituted with one, two or three substituents independently selected from hydrogen, alkyl, alkoxy, hydroxyl, cyano, nitro, halo, haloalkyl, haloalkoxy, alkylthio, alkylsulfonyl, carboxy, alkoxycarbonyl, aminocarbonyl or aminosulfonyl. Preferably, the aryl and heteroaryl rings are selected from:

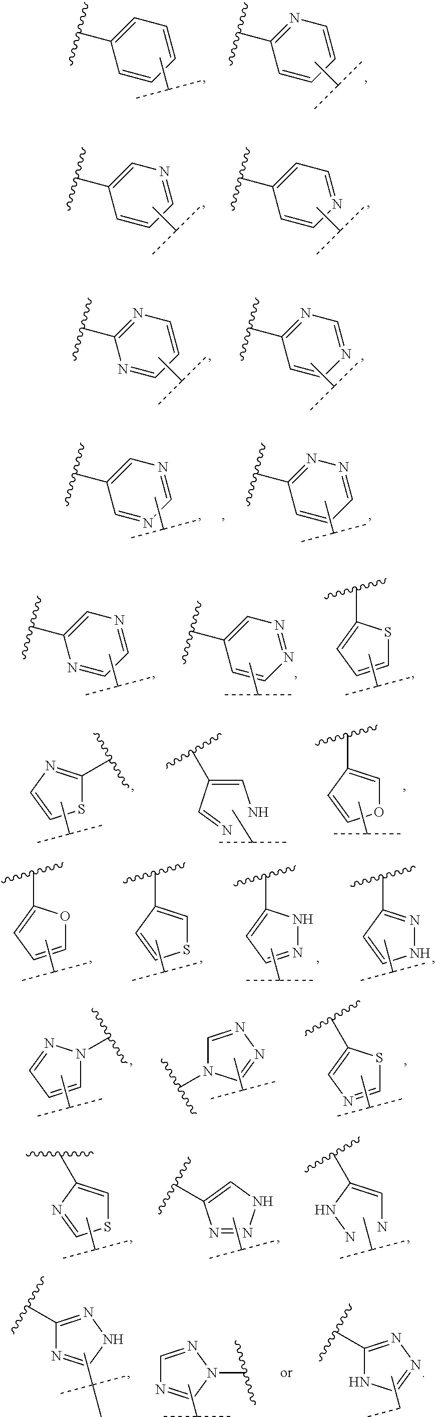

R^{b} is cyano, nitro, fluoro, trifluoromethy, 2,2,2-trifluoroethyl, trifluoromethoxy, 2,2,2-trifluoroethyloxy, methylsulfonyl or methylthio, preferably independently cyano or trifluoromethyl, and R^{c} is methyl, isopropyl, tert-butyl, cyclopropyl, trifluoromethyl, 2,2,2-trifluoroethyl, 1-methyl-1-methylaminoethyl, 1-methyl-1-dimethylaminoethyl, 1-methyl-1-aminoethyl, 1-methylaminocycloprop-1-ylene, or 1-dimethylaminocycloprop-1-ylene.

(iv) With groups in (a), in one group of compounds Z and EWG′ is a bond when Ar is electron withdrawing e.g. pyridinyl.

(v) Within groups in embodiment (I), e.g., Formula I, I[a], I[b], A[a], A[b], B[a], B[b], C[a], C[b], D[a], D[b], I[a,i], I[a,ia], I[a,ii], I[a,iii], I[a,iv], I[b,i], I[b,ia], I[b,ii], I[b,iii], I[b,iv], A[a,i], A[a,ia], A[a,ii], A[a,iii], A[a,iv], A[b,i], AI[b,ia], A[b,ii], A[b,iii], A[b,iv], B[a,i], B[a,ia], B[a,ii], B[a,iii], B[a,iv], B[b,i], B[b,ia], B[b,ii], B[b,iii], B[b,iv], C[a,i], C[a,ia], C[a,ii], C[a,iii], C[a,iv], C[b,i], C[b,ia], C[b,ii], C[b,iii], C[b,iv], D[a,i], D[a,ia], D[a,ii], D[a,iii], D[a,iv], D[b,i], D[b,ia], D[b,ii], D[b,iii], and D[b,iv], and groups contained therein, in one group of compounds R^{b} is cyano.

(vi) Within groups in embodiment (I), e.g., Formula I, I[a], I[b], A[a], A[b], B[a], B[b], C[a], C[b], D[a], D[b], I[a,i], I[a,ia], I[a,ii], I[a,iii], I[a,iv], I[b,i], I[b,ia], I[b,ii], I[b,iii], I[b,iv], A[a,i], A[a,ia], A[a,ii], A[a,iii], A[a,iv], A[b,i], AI[b,ia], A[b,ii], A[b,iii], A[b,iv], B[a,i], B[a,ia], B[a,ii], B[a,iii], B[a,iv], B[b,i], B[b,ia], B[b,ii], B[b,iii], B[b,iv], C[a,i], C[a,ia], C[a,ii], C[a,iii], C[a,iv], C[b,i], C[b,ia], C[b,ii], C[b,iii], C[b,iv], D[a,i], D[a,ia], D[a,ii], D[a,iii], D[a,iv], D[b,i], D[b,ia], D[b,ii], D[b,iii], and D[b,iv], and same combinations for C-D, and groups contained therein, in another group of compounds R^{b} is trifluoromethyl.

(vii) Within groups in embodiment (I), e.g., Formula I, I[a], I[b], A[a], A[b], B[a], B[b], C[a], C[b], D[a], D[b], I[a,i], I[a,ia], I[a,ii], I[a,iii], I[a,iv], I[b,i], I[b,ia], I[b,ii], I[b,iii], I[b,iv], A[a,i], A[a,ia], A[a,ii], A[a,iii], A[a,iv], A[b,i], AI[b,ia], A[b,ii], A[b,iii], A[b,iv], B[a,i], B[a,ia], B[a,ii], B[a,iii], B[a,iv], B[b,i], B[b,ia], B[b,ii], B[b,iii], B[b,iv], C[a,i], C[a,ia], C[a,ii], C[a,iii], C[a,iv], C[b,i], C[b,ia], C[b,ii], C[b,iii], C[b,iv], D[a,i], D[a,ia], D[a,ii], D[a,iii], D[a,iv], D[b,i], D[b,ia], D[b,ii], D[b,iii], and D[b,iv], and same combinations for C-D, and groups contained therein, in another group of compounds R^{b} is methylthio or methylsulfonyl.

### Embodiment J

In another embodiment, within the compound of Formula (I) as defined above and embodiments (A), (B), (C) and/or (D) and groups contained therein:

(a) in one group of compounds:

R¹ is hydrogen, alkyl, hydroxy, alkoxy, halo, haloalkyl, or haloalkoxy and R⁵ is a group of formula (b) and L is NHCONH, NHCO, or CONH.

(b) In another group of compounds:

R⁵ is hydrogen, alkyl, hydroxy, alkoxy, halo, haloalkyl, or haloalkoxy and R¹ is a group of formula (b) and L is O.

Within groups (a) and (b) in embodiment (J), preferably, X^{a} is O, and R^{c′} is methyl, ethyl, propyl, cyclopropyl, or alkylene substituted with hydroxyl, alkoxy, alkylamino or dialkylamino. Preferably, X^{a} is O and R^{c′} is methyl, isopropyl, tert-butyl, cyclopropyl, trifluoromethyl, 2,2,2-trifluoroethyl, 1-methyl-1-methylaminoethyl, 1-methyl-1-dimethylaminoethyl, 1-methyl-1-aminoethyl, 1-methylaminocycloprop-1-ylene, or 1-dimethylaminocycloprop-1-ylene. Within embodiment (I) and groups contained therein, in one group of compounds —P-Q- are as described in embodiment (E) above, and groups contained therein i.e, (c), (d), (i) and (ii) above or combinations thereof. Preferably, P-Q is alkylene, preferably methylene.

### Embodiment K

In another embodiment, within the compound of Formula (I) as defined above and embodiments (A), (B), (C), (D), (E), (F), (G), (H) and/or (I) and groups contained therein, in one group of compounds the

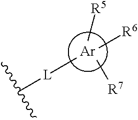

group is attached at the 4-position of the phenyl ring, the carbon atom of the phenyl ring attached to core being carbon 1.

(i) Within the groups in embodiment K, in one group of compounds, Ar is phenyl.

(ii) Within groups in embodiment K, in another group of compounds when R⁵ is not hydrogen, alkyl, alkoxy, halo, haloalkyl, haloalkoxy, or cyano, then, Ar is phenyl substituted at meta or para, preferably meta position with R⁵, and R⁶ is ortho or para to R⁵.

(iii) Within groups in embodiment K, in another group of compounds when R¹ is not hydrogen, alkyl, alkoxy, halo, haloalkyl, haloalkoxy, or cyano, then, Ar is phenyl substituted at meta and/or para with R⁵ or R⁶ which are preferably chloro or trifluoromethyl.

(iv) Within groups in embodiment K, in another group of compounds when R¹ is not hydrogen, alkyl, alkoxy, halo, haloalkyl, haloalkoxy, or cyano, Ar is heteroaryl, preferably pyridyl or pyrimidinyl optionally substituted with R⁵—R⁷.

(v). Within this embodiment, in one group of compounds, Ar is pyrrolidinyl or piperidinyl.

(vi) Within groups in embodiment K, in another group of compounds when R¹ is not hydrogen, alkyl, alkoxy, halo, haloalkyl, haloalkoxy, or cyano, in another group of compounds

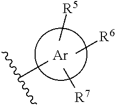

in

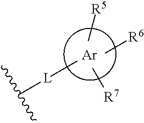

is:

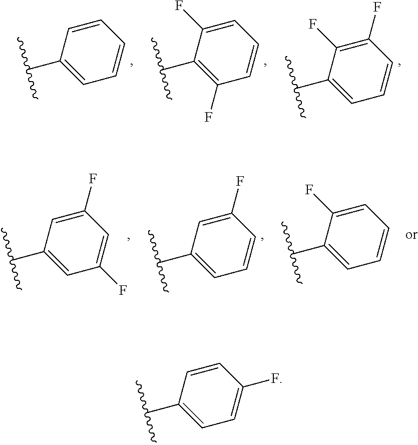

preferably,

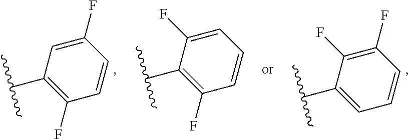

more preferably

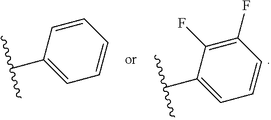 [SMILES: *c1ccccc1 ; *c1cccc(F)c1F]

### Embodiment L

In a further embodiment J, the disclosure includes compounds of embodiments 1-34 below:

1. A compound of Formula (I):

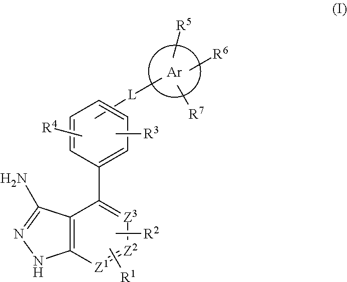

wherein:

Z¹, Z², and Z³ are —N— or CH, provided that not more than two of Z¹, Z², and Z³ are simultaneously N;

L is O, CO, CH₂, S, SO, SO₂, NR, NRCO, CONR, NR′SO₂, SO₂NR′, or NRCONR, where (each R and R′ is independently hydrogen, alkyl, or cycloalkyl);

Ar is aryl, heteroaryl, cycloalkyl or heterocyclyl;

one of R¹ and R⁵ is hydrogen, alkyl, hydroxy, alkoxy, halo, haloalkyl, or haloalkoxy and the other of R¹ and R⁵ is:

(i) —P-Q-CH═C(R^{b})(EWG) where P is a bond, NR^{a} (where R^{a} is hydrogen or alkyl), —O—, S, SO, SO₂, alkylene or heteroalkylene, Q is a bond, aryl or heteroaryl wherein aryl or heteroaryl is optionally substituted with one or two substituents independently selected from hydrogen, halo, alkyl, alkoxy, alkylthio, haloalkyl, or haloalkoxy, R^{b} is cyano, nitro, halo, haloalkyl, haloalkoxy, alkylthio, or alkylsulfonyl and EWG is an electron withdrawing group; or

(ii) —Z-(EWG′)-C(R^{b})═CHR^{c} where Z is bond, NR^{a} (where R^{a} is hydrogen or alkyl), —O—, S, SO, SO₂, alkylene, cycloalkylene, heteroalkylene, -(Z^{a})ₙ₁-aryl, or -(Z^{a})ₙ₁-heteroaryl (wherein n1 is 0 or 1, Z^{a} is NR^{a} (where R^{a} is hydrogen or alkyl), —O—, S, SO, SO₂, alkylene, or heteroalkylene and aryl or heteroaryl is optionally substituted with one or two substituents independently selected from hydrogen, halo, alkyl, alkoxy, alkylthio, haloalkyl, or haloalkoxy), EWG′ is a bond or an electron withdrawing group, R^{b} is cyano, nitro, halo, haloalkyl, haloalkoxy, alkylthio, or alkylsulfonyl and R^{c} is alkyl, substituted alkyl, haloalkoxy, cycloalkyl, cycloalkyleneNR^{d}R^{e} where R^{d} and R^{e} are independently hydrogen, alkyl, or cycloalkyl; or

(iii) a group of formula (a) or (b);

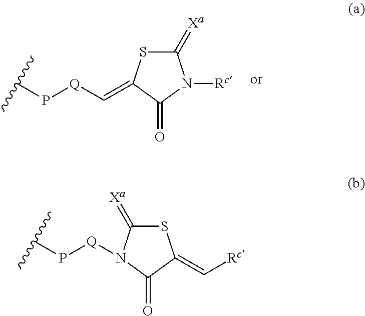

where P and Q are as defined above, X^{a} is O, S, or N(H or alkyl) and R^{c′} is hydrogen, alkyl, substituted alkyl, haloalkoxy, cycloalkyl, or cycloalkyleneNR^{d}R^{e} where R^{d} and R^{e} are independently hydrogen, alkyl, or cycloalkyl;

R² is hydrogen, alkyl, hydroxy, alkoxy, cyano, halo or haloalkyl;

R³ and R⁴ are independently hydrogen, alkyl, cycloalkyl, hydroxy, alkoxy, cyano, halo, haloalkyl or haloalkoxy; and

R⁶ and R⁷ are independently hydrogen, alkyl, hydroxy, alkoxy, halo, haloalkyl, haloalkoxy, carboxy, alkoxycarbonyl, cyano, —CONH₂, amino, monosubstituted and disubstituted amino;

or a pharmaceutically acceptable salt thereof;

2. The compound of previous embodiment 1 wherein:

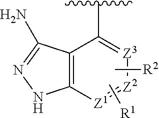

is

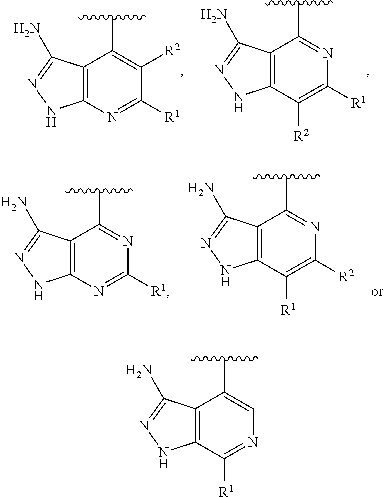

3. The compound of previous embodiment 1 or 2 wherein:

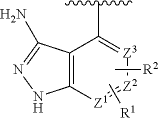

is

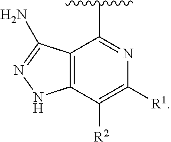

4. The compound of previous embodiment of 1 or 2 wherein:

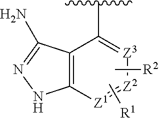

is

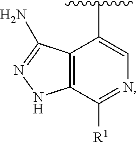

5. The compound of previous embodiment 1 or 2 wherein:

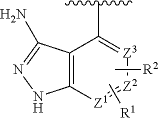

is

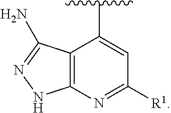

6. The compound of any of the previous embodiments 1-5 above wherein L is O, S, NH, or N(methyl), NHCO, CONH, or NHCONH.

7. The compound of any of the previous embodiments 1-6 above wherein R³ and R⁴ are independently hydrogen, alkyl, alkoxy, cyano, halo, haloalkyl or haloalkoxy, preferably independently hydrogen, methyl, fluoro, methoxy, chloro, trifluoromethyl, or trifluoromethoxy.

8. The compound of any of the previous embodiments 1-7 above wherein R⁶ and R⁷ are independently hydrogen, alkyl, alkoxy, halo, haloalkyl, haloalkoxy, or cyano, preferably hydrogen, methyl, methoxy, fluoro, chloro, trifluoromethyl, trifluoromethoxy, or cyano.

9. The compound of any of the previous embodiments 1-8 above wherein R⁵ is hydrogen, alkyl, alkoxy, halo, haloalkyl, haloalkoxy, or cyano, preferably, R⁵ is hydrogen, methyl, methoxy, fluoro, chloro, trifluoromethyl, trifluoromethoxy, or cyano; and
R¹ is —P-Q-CH═C(R^{b})(EWG) where P is a bond, NR^{a} (where R^{a} is hydrogen or alkyl), —O—, S, SO, SO₂, or alkylene, Q is a bond, aryl or heteroaryl, R^{b} is cyano, nitro, halo, haloalkyl, haloalkoxy, alkylthio, or alkylsulfonyl, EWG is an electron withdrawing group; and L is O.

10. The compound of any of the previous embodiments 1-8 above wherein R¹ is hydrogen, alkyl, alkoxy, halo, haloalkyl, haloalkoxy, or cyano, preferably, R¹ is hydrogen, methyl, methoxy, fluoro, chloro, trifluoromethyl, trifluoromethoxy, or cyano; and
R⁵ is —P-Q-CH═C(R^{b})(EWG) where P is a bond, NR^{a} (where R^{a} is hydrogen or alkyl), —O—, S, SO, SO₂, alkylene or heteroalkylene, Q is a bond, aryl or heteroaryl, R^{b} is cyano, nitro, halo, haloalkyl, haloalkoxy, alkylthio, or alkylsulfonyl, EWG is an electron withdrawing group; and L is NHCONH, NHCO, or CONH.

11. The compound of any of the previous embodiment 9 or 10 above wherein:
—P— is a bond, NR^{a}, O, or methylene and Q is aryl or heteroaryl, preferably, Q is selected from:

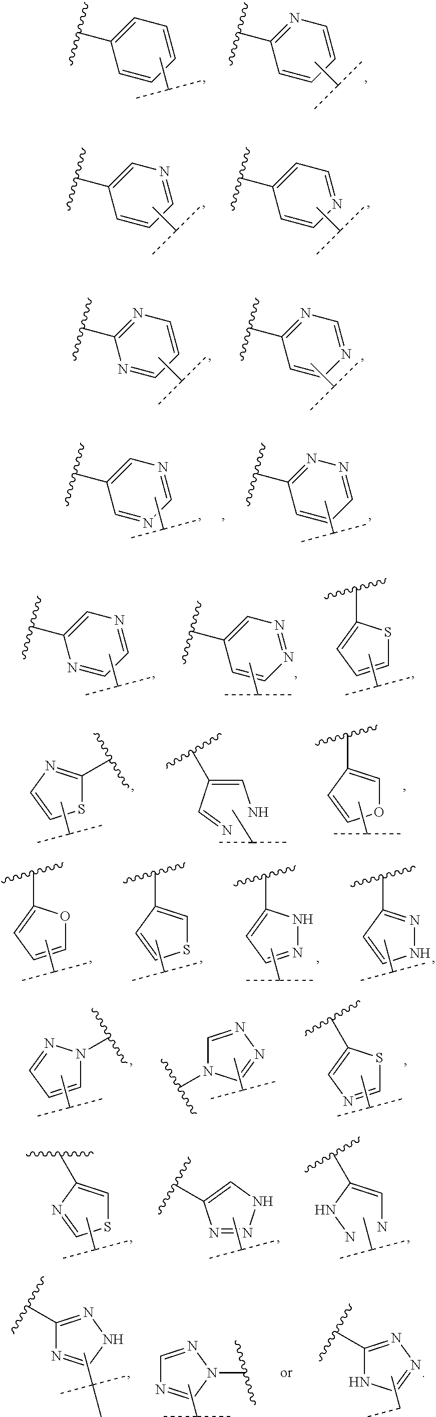

each substituted with one, two or three substituents independently selected from hydrogen, alkyl, alkoxy, hydroxyl, cyano, nitro, halo, haloalkyl, haloalkoxy, alkylthio, alkylsulfonyl, carboxy, alkoxycarbonyl, aminocarbonyl or aminosulfonyl. Preferably when Q is heteroaryl wherein heteroaryl ring is six membered ring shown above, then P is a bond, O, or NR^{a}, preferably a bond and when Q is a five membered ring, P is methylene.

12. The compound of any of the previous embodiments 9 or 10 above wherein R^{b} is cyano.

13. The compound of any of the previous embodiments 9 or 10 above wherein R^{b} is trifluoromethyl.

14. The compound of any of the previous embodiments 9 or 10 above wherein R^{b} is nitro, methylthio or methylsulfonyl.

15. The compound of any of the previous embodiments 1-14 above wherein EWG is —N(R′₃)⁺, —SO₃H, —SO₃R′, —S(O₂)R′, —S(O)R′, —C(O)NH₂, —C(O)NHR^{g}, —C(O)NR^{f}R^{g}, —S(O₂)NH₂, —SO₂NHRⁱ′, —SO₂NR^{h}Rⁱ, —PO(OR′)₂, —PO₃H₂, —PO(NR′₂)₂, —C≡N, —CH(haloalkyl), —C(O)X′, —COOH, —COOR′, —C(O)R′, —C(O)H, —P(O)(OR′)OR″, halo, heteroaryl, or aryl wherein X′ is independently halogen (e.g. chloro of fluoro), and R′, R″, R^{f}, R^{g}, R^{h}, and Rⁱ are independently hydrogen, alkyl, substituted alkyl, cycloalkyl, cycloalkyleneNR^{d}R^{e} (where R^{d} and R^{e} are independently hydrogen, alkyl, or cycloalkyl) or R^{f} and R^{g} and R^{h} and Rⁱ together with the nitrogen atom to which they are attached form heterocycloamino; and aryl and heteroaryl are substituted with one, two or three substituents independently selected from hydrogen, alkyl, alkoxy, hydroxyl, cyano, nitro, halo, haloalkyl, haloalkoxy, alkylthio, alkylsulfonyl, carboxy, alkoxycarbonyl, aminocarbonyl or aminosulfonyl.

16. The compound of any of the previous embodiments 1-14 above wherein EWG is —CO—NR^{f}R^{g} or —SO₂NR^{h}Rⁱ (wherein R^{f} and R^{h} are independently hydrogen, alkyl, or cycloalkyl and R^{g} and Rⁱ are independently hydrogen, alkyl, substituted alkyl, cycloalkyleneNR^{d}R^{e} (where R^{d} and R^{e} are independently hydrogen, alkyl, or cycloalkyl; or R^{d} and R^{e} together with the nitrogen atom to which they are attached form heterocycloamino), or

R^{f} and R^{g} and R^{h} and Rⁱ together with the nitrogen atom to which they are attached form heterocycloamino) and aryl or heteroaryl wherein each of the aforementioned ring is substituted with one, two or three substituents independently selected from hydrogen, alkyl, alkoxy, hydroxyl, cyano, nitro, halo, haloalkyl, haloalkoxy, alkylthio, alkylsulfonyl, carboxy, alkoxycarbonyl, aminocarbonyl or aminosulfonyl. Preferably, in one group of compounds EWG is pyridine-2-yl, pyridine-3-yl, pyridine-4-yl, pyrrol-1-yl, pyrazol-1-yl, or thiazol-2-yl. Preferably, in another group of compounds EWG is dimethylaminocarbonyl, methylaminocarbonyl, isopropylaminocarbonyl, tert-butylaminocarbonyl, or 3-hydroxy-1-methylpropylaminocarbonyl. Preferably, in yet another group of compounds EWG is azetidin-1-ylcarbonyl, 4-hydroxyazetidin-1-ylcarbonyl, pyrrolidin-1-ylcarbonyl, 4-ethylpiperazin-1-ylcarbonyl, or 2,6-dimethylmorpholine-4-ylcarbonyl.

17. The compound of any of the previous embodiments 1-14 above wherein EWG is —CON(CH₃)₂, or

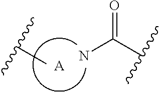

where A is heterocycloamino (such as pyrrolidnyl, piperazinyl or piperidinyl) optionally substituted with hydroxyl, methyl, methoxy, amino, methylamino or dimethylamino, preferably substituted at the 3 or 4 position of the piperidinyl and piperazinyl rings.

18. The compound of any of the previous embodiments 1-14 above wherein EWG is selected from

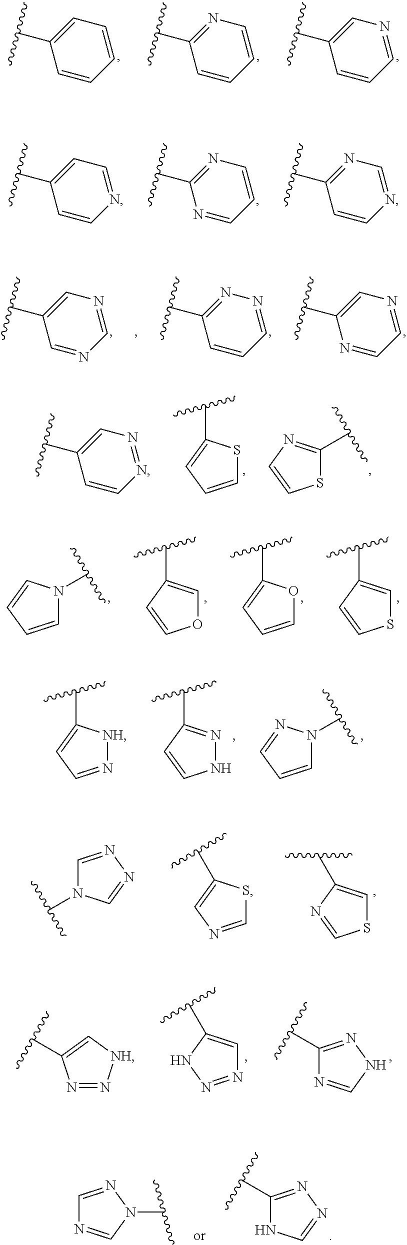

each ring independently selected from and is substituted with one, two or three substituents independently selected from hydrogen, halo, haloalkyl, cyano, haloalkoxy, or alkylsulfonyl, preferably hydrogen, fluoro, cyano, trifluoromethyl, trifluoromethoxy, or cyano.

19. The compound of any of the previous embodiments 1-8 above wherein:
R⁵ is hydrogen, alkyl, alkoxy, halo, haloalkyl, haloalkoxy, or cyano; preferably, R⁵ is hydrogen, methyl, methoxy, fluoro, chloro, trifluoromethyl, trifluoromethoxy, or cyano;
R¹ a group of formula (a)

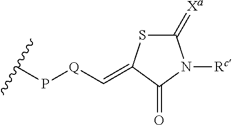

where X^{a} is O or N(H or alkyl) and R^{c′} is hydrogen, alkyl, cycloalkyl, substituted alkyl or cycloalkyleneNR^{d}R^{e} and L is O.

20. The compound of any of the previous embodiments 1-8 above wherein:
R¹ is hydrogen, alkyl, alkoxy, halo, haloalkyl, haloalkoxy, or cyano. Preferably, R¹ is hydrogen, methyl, methoxy, fluoro, chloro, trifluoromethyl, trifluoromethoxy, or cyano; and
R⁵ a group of formula (a)

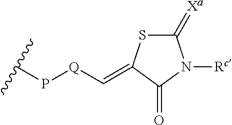

where X^{a} is O or N(H or alkyl) and R^{c′} is hydrogen, alkyl, cycloalkyl, substituted alkylalkyl or cycloalkyleneNR^{d}R; and L is NHCONH, NHCO, or CONH.

21. The compound of any of the previous embodiment 19 or 20 above wherein:
—P— is a bond, NR^{a}, O, or methylene and Q is bond, aryl or heteroaryl, preferably, Q is bond or is selected from:

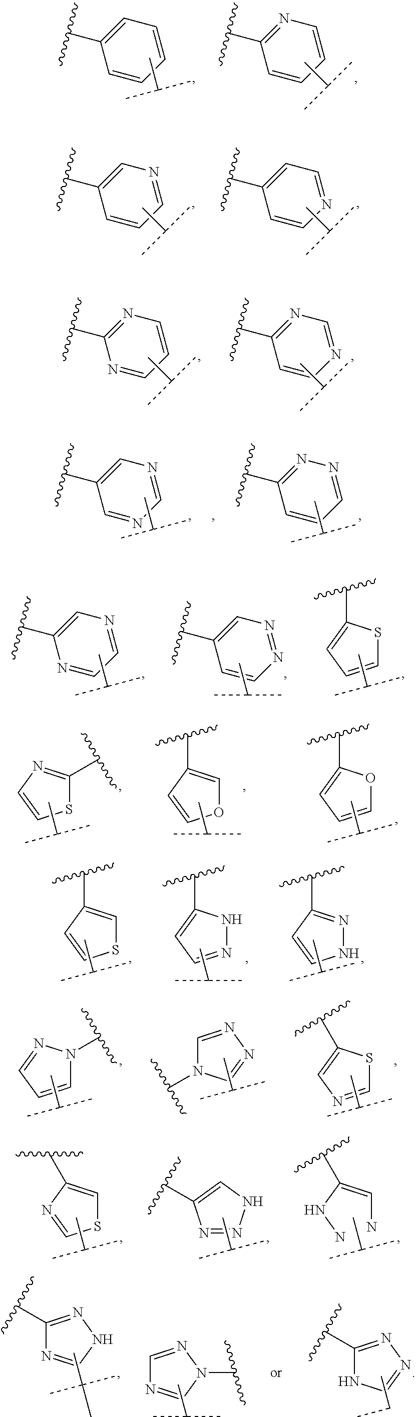

each substituted with one, two or three substituents independently selected from hydrogen, alkyl, alkoxy, hydroxyl, cyano, nitro, halo, haloalkyl, haloalkoxy, alkylthio, alkylsulfonyl, carboxy, alkoxycarbonyl, aminocarbonyl or aminosulfonyl. Preferably,
P is methylene and Q is a bond.

22. The compound of any of the previous embodiments 1-8 above wherein:
R¹ is hydrogen, alkyl, hydroxy, alkoxy, halo, haloalkyl, or haloalkoxy; and
R⁵ is —Z-(EWG′)-C(R^{b})═CHR^{c} where Z is bond, NR^{a} (where R^{a} is hydrogen or alkyl) —O—, S, SO, SO₂, alkylene, phenyl, heteroaryl, or heteroalkylene, EWG′ is an electron withdrawing group, R^{b} is cyano, nitro, halo, haloalkyl, haloalkoxy, alkylthio, or alkylsulfonyl and R^{c} is alkyl, substituted alkyl, cycloalkyl, cycloalkyleneNR^{d}R^{e} where R^{d} and R^{e} are independently hydrogen, alkyl, or cycloalkyl; and
L is NHCONH, NHCO, or CONH.

23. The compound of any of the previous embodiments 1-8 above wherein:
R⁵ is hydrogen, alkyl, hydroxy, alkoxy, halo, haloalkyl, or haloalkoxy;
R¹ is —Z-(EWG′)-C(R^{b})═CHR^{c} where Z is bond, NR^{a} (where R^{a} is hydrogen or alkyl), —O—, S, SO, SO₂, alkylene, phenyl, heteroaryl, or heteroalkylene, EWG′ is an electron withdrawing group, R^{b} is cyano, nitro, halo, haloalkyl, haloalkoxy, alkylthio, or alkylsulfonyl and R^{c} is alkyl, substituted alkyl, cycloalkyl, cycloalkyleneNR^{d}R^{e} where R^{d} and R^{e} are independently hydrogen, alkyl, or cycloalkyl; and
L is O.

24. The compound of any of the previous embodiment 22 or 23 above wherein:
Z is bond, NR^{a}, O or alkylene; EWG′ is

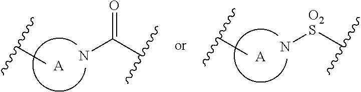

where ring A is heterocycloamino; preferably Z-EWG′ is 3-piperidin-1-ylcarbonyl or 2-methylenepyrrolidin-1-ylcarbonyl;

R^{b} is cyano, nitro, fluoro, trifluoromethyl, 2,2,2-trifluoroethyl, trifluoromethoxy, 2,2,2-trifluoroethyloxy, methylsulfonyl or methylthio, trifluoromethyl and R^{c} is methyl, isopropyl, tert-butyl, cyclopropyl, trifluoromethyl, 2,2,2-trifluoroethyl, 1-methyl-1-methylaminoethyl, 1-methyl-1-dimethylaminoethyl, 1-methyl-1-aminoethyl, 1-methylaminocycloprop-1-ylene, or 1-dimethylaminocycloprop-1-ylene. Preferably, R^{b} is cyano. Preferably, R^{b} is trifluoromethyl.

25. The compound of any of the previous embodiment 22 or 23 above wherein:

Z is bond, cycloalkylene, phenyl, heteroaryl, or alkylene and EWG′ is —NR′CO— or —NR′ SO₂;

wherein each R′ is independently hydrogen or alkyl; R^{b} is cyano, nitro, fluoro, trifluoromethy, 2,2,2-trifluoroethyl, trifluoromethoxy, 2,2,2-trifluoroethyloxy, methylsulfonyl or methylthio and R^{c} is methyl, isopropyl, tert-butyl, cyclopropyl, trifluoromethyl, 2,2,2-trifluoroethyl, 1-methyl-1-methylaminoethyl, 1-methyl-1-dimethylaminoethyl, 1-methyl-1-aminoethyl, 1-methylaminocycloprop-1-ylene, or 1-dimethylaminocycloprop-1-ylene.

Preferably, EWG′ is —NHCO— and Z is

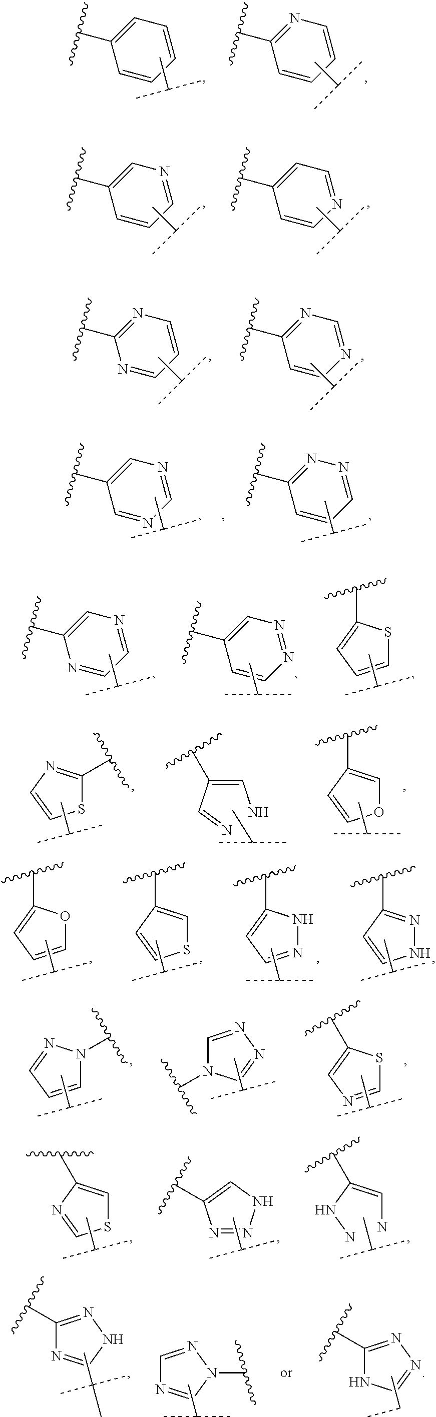

preferably phenyl. Preferably, R^{b} is cyano. Preferably, R^{b} is trifluoromethyl.

26. The compound of any of the previous embodiment 22 or 23 above wherein:
Z is bond, NR^{a}, O or alkylene and
EWG′ is heteroaryl, or aryl; wherein R^{a} is independently hydrogen or alkyl; and aryl and heteroaryl are substituted with one, two or three substituents independently selected from hydrogen, alkyl, alkoxy, hydroxyl, cyano, nitro, halo, haloalkyl, haloalkoxy, alkylthio, alkylsulfonyl, carboxy, alkoxycarbonyl, aminocarbonyl or aminosulfonyl.

27. The compound of any of the previous embodiments 1-8 above, wherein:
R¹ is hydrogen, alkyl, hydroxy, alkoxy, halo, haloalkyl, or haloalkoxy and R⁵ is a group of formula (b) and L is NHCONH, NHCO, or CONH.

28. The compound of any of the previous embodiments 1-8 above, wherein:
R⁵ is hydrogen, alkyl, hydroxy, alkoxy, halo, haloalkyl, or haloalkoxy and R¹ is a group of formula (b) and L is O.

29. The compound of any of the previous embodiment 27 or 28 above wherein:
X^{a} is O, and R^{c′} is methyl, ethyl, propyl, cyclopropyl, or alkylene substituted with hydroxyl, alkoxy, alkylamino or dialkylamino; preferably, X^{a} is O, and
R^{c′} is methyl, isopropyl, tert-butyl, cyclopropyl, trifluoromethyl, 2,2,2-trifluoroethyl, 1-methyl-1-methylaminoethyl, 1-methyl-1-dimethylaminoethyl, 1-methyl-1-aminoethyl, 1-methylaminocycloprop-1-ylene, or 1-dimethylaminocycloprop-1-ylene.

30. The compound of any of the previous embodiments 1-29 above, wherein

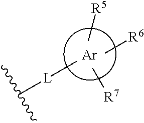

group is attached at the 4-position of the phenyl ring.

31. The compound of any of the previous embodiments 1-30 above, wherein

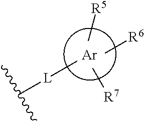

phenyl.

32. The compound of any of the previous embodiments 1-30 above, wherein

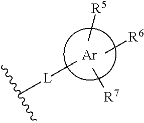

heteroaryl.

### Embodiment M

In yet another embodiment, the compound of Formula (I) has the structure (Ia) shown below:

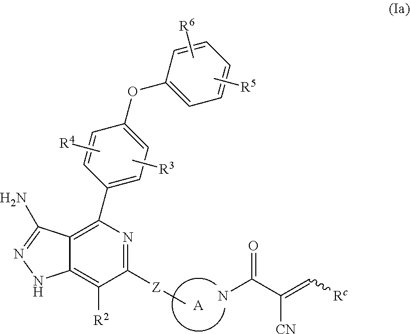

wherein:

R² is hydrogen or alkyl;

R³ and R⁴ are independently hydrogen, alkyl, haloalkyl, fluoro or chloro;

R⁵ and R⁶ are independently hydrogen or fluoro;

Z is a bond or alkylene;

ring A in

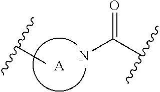

is heterocycloamino optionally substituted with one or two alkyl; and

R^{c} is cycloalkyl, alkyl, substituted alkyl, cycloalkyleneNR^{d}R^{e} or cycloalkylene(alkylene)NR^{d}R^{e} (where R^{d} and R^{e} are independently hydrogen, alkyl, or cycloalkyl) or 3 to 6 membered saturated monocyclic heterocyclyl containing one or two heteroatoms selected from N, O, or S and optionally substituted with one or two substituents selected from hydroxy, alkyl or fluoro.

(i) Within embodiment (M), in one group of compounds:

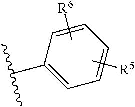

is a ring of formula:

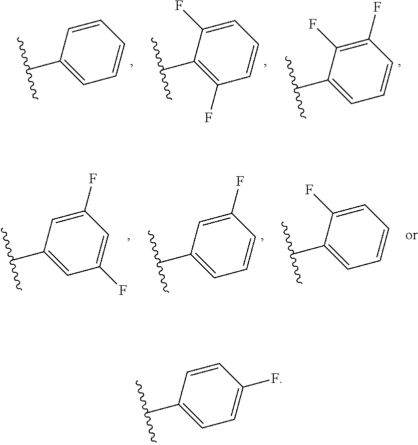

### Preferably,

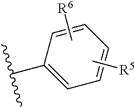

is a ring of formula:

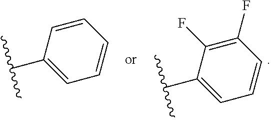 [SMILES: *c1ccccc1 ; *c1cccc(F)c1F]

Preferably, in one group of compound

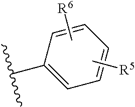

is phenyl. Preferably, in another group of compound

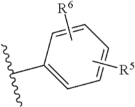

is

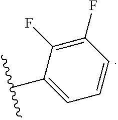 [SMILES: *c1cccc(F)c1F]

(ii) Within embodiment (M) and subpart (a) of Embodiment (M) and groups contained therein, in one group of compounds:

R² is hydrogen or alkyl, preferably hydrogen or methyl, more preferably hydrogen;

R³ and R⁴ are independently hydrogen, methyl, ethyl, trifluoromethyl, fluoro or chloro. Preferably, within groups in subpart (ii), in one group of compounds

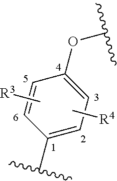

is a ring of formula:

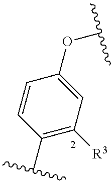

where R³ is hydrogen, methyl, ethyl, chloro, fluoro or trifluoromethyl, preferably hydrogen, methyl, ethyl, chloro or fluoro, more preferably, hydrogen, methyl, fluoro, or chloro, even more preferably hydrogen, chloro or fluoro, particularly preferably hydrogen or fluoro. Preferably, within groups in subpart (ii), in another group of compounds

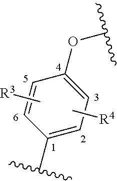

is a ring of formula

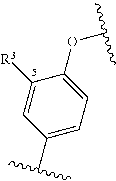

where R³ is alkyl or halo, preferably methyl, chloro or fluoro. Preferably, within groups in subpart (ii), in yet another group of compounds

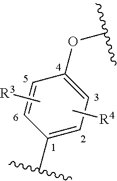

is a ring of formula

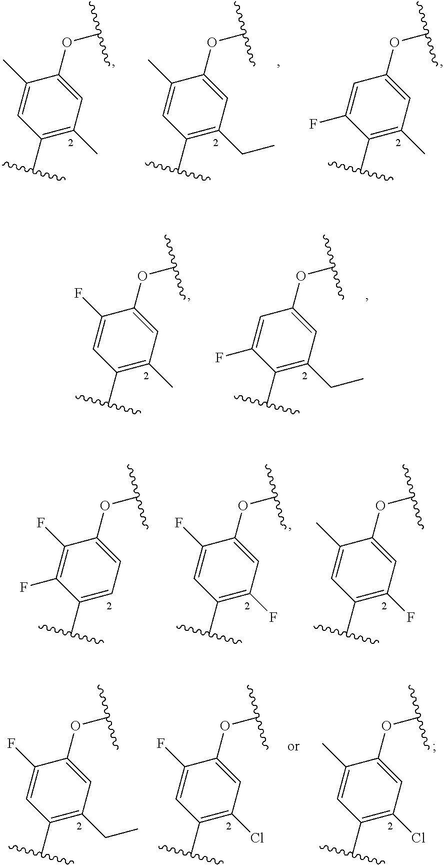

preferably,

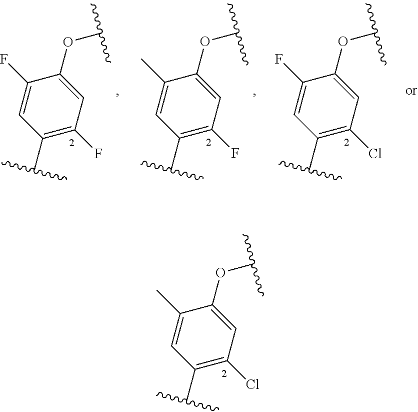 [SMILES: *Oc1cc(F)c(*)cc1F ; *Oc1cc(F)c(*)cc1C ; *Oc1cc(Cl)c(*)cc1F ; *Oc1cc(Cl)c(*)cc1C]

(iii) Within embodiment (M) and groups contained therein, and subpart (i) and/or (ii) of Embodiment (M) and groups contained therein, in one group of compounds ring A in

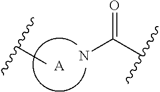

is pyrrolinyl or piperidinyl, each ring optionally substituted with one or two alkyl, preferably methyl. Within the groups in subpart (iii), in one group of compounds

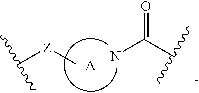

is 3-piperidin-1-carbonyl (ie, the piperidin-1-yl ring is attached via the C-3 carbon of the piperidinyl ring to the C6 position of 1H-pyrazolo[4,3-c]pyridin-3-amine ring). Within the groups in subpart (iii), in another group of compounds

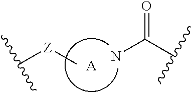

is 2-CH₂-pyrrolidin-1-ylcarbonyl, 2-CH(CH₃)-pyrrolidin-1-ylcarbonyl; 2-CH₂-3,3-dimethylpyrrolidin-1-ylcarbonyl or 2-CH₂-4,4-dimethylpyrrolidin-1-ylcarbonyl, the carbon atom of the pyrrolidinyl ring attached to —CH₂— having (R) or (S) stereochemistry. More preferably,

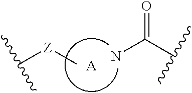

is 2-CH₂-pyrrolidin-1-ylcarbonyl (ie, the 2-CH₂-pyrrolidin-1-ylcarbonyl ring is attached to the C6 position of 1H-pyrazolo[4,3-c]pyridin-3-amine ring via the CH₂ group which is located at C2 position of the pyrrolidinyl ring).

(a) Within embodiment (M) and groups contained therein, and subpart (i) and/or (ii) and/or (iii) and groups contained therein, in one group of compounds R^{c} is cycloalkyl, alkyl, or substituted alkyl, preferably, cyclopropyl, isopropyl, tert-butyl, 1-methyl-1-methylaminoethyl, 1-methyl-1-dimethylaminoethyl, 1-methyl-1-aminoethyl or 1-ethoxy-1-methylethyl. Within the groups in (a), in one group of compounds R^{c} is isopropyl. Within the groups in (a), in another group of compounds R^{c} is tert-butyl. Within the groups in (a), in one group of compounds R^{c} is 1-methyl-1-methylaminoethyl, 1-methyl-1-dimethylaminoethyl, or 1-methyl-1-aminoethyl, preferably 1-methyl-1-aminoethyl.

(b) Within embodiment (M) and groups contained therein, and subpart (i) and/or (ii) and/or (iii) and groups contained therein, in one group of compounds R^{c} is cycloalkyl, preferably cyclopropyl.

(c) Within embodiment (M) and groups contained therein, and subpart (i) and/or (ii) and/or (iii) and groups contained therein, in one group of compounds R^{c} is alkyl, preferably isopropyl or tert-butyl, more preferably isopropyl.

(d) Within embodiment (M) and groups contained therein, and subpart (i) and/or (ii) and/or (iii) and groups contained therein, in one group of compounds R^{c} is substituted alkyl, preferably, alkyl substituted with alkoxy or NRR′ (where R is hydrogen, alkyl, alkoxyalkyl or cycloalkyl and R′ is hydrogen or alkyl), or heterocyclcyl which is optionally substituted with one or two groups independently selected from alkyl), preferably R^{c} is —C(CH₃)₂NH₂, —C(CH₃)₂NHCH₃, —C(CH₃)₂N(CH₃)₂, —C(CH₃)₂NHCH₂CH₃, —C(CH₃)₂NHCH(CH₃)₂, —C(CH₃)₂NHcyclopropyl, —C(CH₃)₂NH(CH₂)₂OCH₃, —C(CH₃)₂OCH₂CH₃, —C(CH₃)₂-morpholine-4-yl. Within groups in (d), in one group of compounds R^{c} is —C(CH₃)₂NH₂, —C(CH₃)₂NHCH₃, —C(CH₃)₂N(CH₃)₂, —C(CH₃)₂NHCH₂CH₃, —C(CH₃)₂NHCH(CH₃)₂ or —C(CH₃)₂NHCH₂OCH₃. Within groups in (d), in another group of compounds R^{c} is —C(CH₃)₂NHcyclopropyl. Within groups in (d), in yet another group of compounds R^{c} is —C(CH₃)₂OCH₂CH₃. Within groups in (d), in yet another group of compounds R^{c} is —C(CH₃)₂-morpholine-4-yl. Within groups in (d), in yet another group of compounds R^{c} is —C(CH₃)₂NH₂.

(e) Within embodiment (M) and groups contained therein, and subpart (i) and/or (ii) and/or (iii) and groups contained therein, in one group of compounds R^{c} is cycloalkylene(alkylene)NR^{d}R^{e} (where R^{d} and R^{e} are independently hydrogen, alkyl, or cycloalkyl), preferably

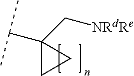

where n is 1-3, R^{d} is hydrogen, methyl or ethyl, and R^{e} is hydrogen, methyl, ethyl, or isopropyl.

(f) Within embodiment (M) and groups contained therein, and subpart (i) and/or (ii) and/or (iii) and groups contained therein, in one group of compounds R^{c} is cycloalkyleneNR^{d}R^{e} (where R^{d} and R^{e} are independently hydrogen, or alkyl), preferably

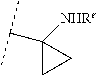

where R^{e} is hydrogen, methyl, ethyl or isopropyl.

(g) Within embodiment (M) and groups contained therein, and subpart (i) and/or (ii) and/or (iii) and groups contained therein, in one group of compounds R^{c} is 3 to 6 membered saturated monocyclic heterocyclyl containing one or two heteroatoms selected from N, O, or S and optionally substituted with one or two substituents selected from hydroxy, alkyl or fluoro; preferably pyrrolidinyl, piperidinyl, tetrahydrofuranyl, or tetrahydropyranyl, more preferably 2-pyrrolidinyl, 3- or 4-piperidinyl, 1-methylpiperidin-4-yl, 1-methylpiperidin-3-yl, or 4-tetrahydropyranyl.

### Embodiment N

In a further embodiment N, the disclosure includes compounds of embodiments 1-23 below:

1. A compound of Formula (I′):

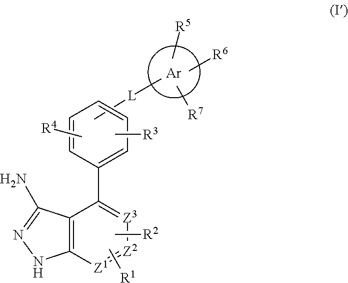

wherein:

Z¹, Z², and Z³ are —N— or CH, provided that not more than two of Z¹, Z², and Z³ are simultaneously N;

L is —O—, —C(O)—, —CH₂—, —S—, —S(O)—, —S(O₂)—, —N(R)—, —N(R)C(O)—, —C(O)N(R)—, —N(R′)S(O₂)—, —S(O₂)N(R′)—, or —N(R)C(O)N(R′)—, where each R and R′ is independently hydrogen, alkyl or cycloalkyl;

Ar is aryl, heteroaryl, cycloalkyl or heterocyclyl;

one of R¹ and R⁵ is hydrogen, alkyl, hydroxy, alkoxy, halo, haloalkyl, or haloalkoxy and the other of R¹ and R⁵ is —Z-(EWG′)-C(R^{b})═CHR^{c} where Z is bond, NR^{a} (where R^{a} is hydrogen or alkyl), —O—, —S—, —S(O)—, —S(O₂)— alkylene, cycloalkylene, heteroalkylene, -(Z^{a})ₙ₁-aryl, or -(Z^{a})ₙ₁-heteroaryl (wherein n1 is 0 or 1, Z^{a} is NR^{a} (where R^{a} is hydrogen or alkyl), —O—, S, SO, SO₂, alkylene, or heteroalkylene and the aryl or heteroaryl is optionally substituted with one or two substituents independently selected from hydrogen, halo, alkyl, alkoxy, alkylthio, haloalkyl, or haloalkoxy), EWG′ is a bond, —CH(haloalkyl), —NR′, —S(O₂)—, —S(O)—, —CO—, —NR′CO—, —NR′SO₂—,

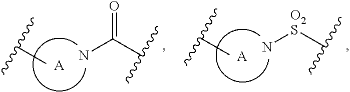

heteroaryl, or aryl; wherein each R′ is independently hydrogen, alkyl, substituted alkyl, or cycloalkyl; ring A is heterocycloamino where the carbonyl and sulfonyl groups are attached to —C(R^{b})═CHR^{c}; and unless defined otherwise, the heterocycloamino, aryl and heteroaryl are optionally substituted with one, two or three substituents independently selected from hydrogen, alkyl, alkoxy, hydroxyl, cyano, nitro, halo, haloalkyl, haloalkoxy, alkylthio, alkylsulfonyl, carboxy, alkoxycarbonyl, aminocarbonyl or aminosulfonyl, R^{b} is cyano, nitro, halo, haloalkyl, haloalkoxy, alkylthio, or alkylsulfonyl and R^{c} is alkyl, substituted alkyl, haloalkoxy, cycloalkyl, cycloalkyleneNR^{d}R^{e} or cycloalkylene(alkylene)NR^{d}R^{e} (where R^{d} and R^{e} are independently hydrogen, alkyl, or cycloalkyl) or 3 to 6 membered saturated monocyclic heterocyclyl containing one or two heteroatoms selected from N, O, or S and optionally substituted with one or two substituents selected from hydroxy, alkyl or fluoro;

R² is hydrogen, alkyl, hydroxy, alkoxy, cyano, halo or haloalkyl;

R³ is hydrogen, alkyl, cycloalkyl, hydroxy, alkoxy, cyano, halo, haloalkyl or haloalkoxy;

R⁴ is hydrogen, alkyl, alkynyl, cycloalkyl, alkylamino, dialkylamino, alkylthio, alkylsulfonyl, carboxy, alkoxycarbonyl, alkylaminosulfonyl, dialkylaminosulfonyl, —CONH₂, alkylaminocarbonyl, dialkylaminocarbonyl, 3, 4 or 5 membered monocyclic heterocyclyl, hydroxy, alkoxy, cyano, halo, haloalkyl or haloalkoxy; and

R⁶ and R⁷ are independently hydrogen, alkyl, hydroxy, alkoxy, halo, haloalkyl, haloalkoxy, carboxy, alkoxycarbonyl, cyano, —CONH₂, amino, or monosubstituted and disubstituted amino;

or a pharmaceutically acceptable salt thereof.

2. The compound of previous embodiment 1 of Embodiment N, wherein:

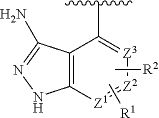

is

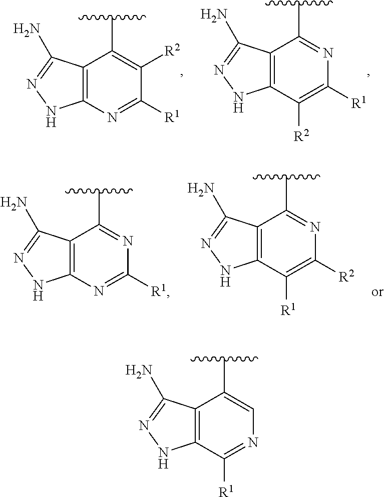

3. The compound of previous embodiment 1 of Embodiment N wherein:

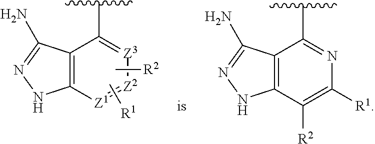

4. The compound of previous embodiment 1 of Embodiment N wherein:

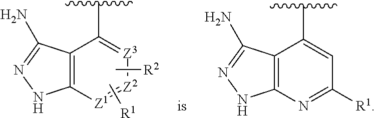

5. The compound of any of the previous embodiments 1-4 of Embodiment N wherein L is O, S, NH, or N(methyl), NHCO, CONH, or NHCONH.
6. The compound of any of the previous embodiments 1-4 of Embodiment N wherein L is O.
7. The compound of any of the previous embodiments 1-4 of Embodiment N wherein L is NHCONH.
8. The compound of any of the previous embodiments 1-7 of Embodiment N wherein R³ and R⁴ are independently hydrogen, alkyl, alkoxy, cyano, halo, haloalkyl or haloalkoxy.
9. The compound of any of the previous embodiments 1-7 of Embodiment N wherein R³ and R⁴ are independently hydrogen, methyl, fluoro, methoxy, chloro, trifluoromethyl, or trifluoromethoxy.
10. The compound of any of the previous embodiments 1-7 of Embodiment N wherein

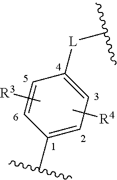

is a ring of formula:

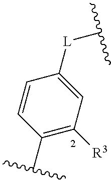

where R³ is hydrogen, methyl, ethyl, chloro, fluoro or trifluoromethyl.
11. The compound of any of the previous embodiments 1-7 of Embodiment N wherein

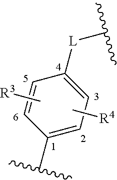

is a ring of formula:

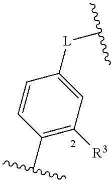

where R³ is hydrogen or fluoro. Within the groups in embodiment 11, in one group of compounds R³ is hydrogen. Within the groups in embodiment 11, in another group of compounds R³ is fluoro.
12. The compound of any of the previous embodiments 1-11 of Embodiment N wherein:

R⁵ is hydrogen, alkyl, hydroxy, alkoxy, halo, haloalkyl, or haloalkoxy;

R¹ is —Z-(EWG′)-C(R^{b})═CHR^{c}; and

L is O.

13. The compound of any of the previous embodiments 1-12 of Embodiment N where Z is bond or alkylene, EWG′ is —NR′CO—, —NR′SO₂—,

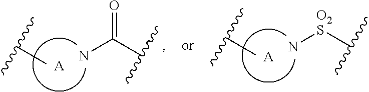

and R^{c} is alkyl, substituted alkyl, haloalkoxy, cycloalkyl, cycloalkyleneNR^{d}R^{e} or 3 to 6 membered saturated monocyclic heterocyclyl containing one or two heteroatoms selected from N, O, or S and optionally substituted with one or two substituents selected from hydroxy, alkyl or fluoro. Within the groups in embodiment 11, in one group of compounds, R^{c} is alkyl. Within the groups in embodiment 11, in another group of compounds, R^{c} is cycloalkyl. Within the groups in embodiment 11, in another group of compounds, R^{c} is alkyl substituted with hydroxy, alkoxy, —NRR′ (where R is hydrogen, alkyl, hydroxyalkyl, or alkoxyalkyl, and R′ is hydrogen, alkyl, or cycloalkyl) or heterocyclyl (preferably heterocycloamino) optionally substituted with one or two groups independently selected from alkyl. Within the groups in embodiment 11, in another group of compounds, R^{c} is alkyl substituted with hydroxy or alkoxy. Within the groups in embodiment 11, in another group of compounds, R^{c} is alkyl substituted with —NRR′ (where R is hydrogen or and R′ is hydrogen or alkyl). Within the groups in embodiment 11, in another group of compounds, R^{c} is alkyl substituted with heterocycloamino optionally substituted with one or two alkyl.
14. The compound of any of the previous embodiments 1-13 of Embodiment N wherein R⁶ and R⁷ are independently hydrogen, alkyl, alkoxy, halo, haloalkyl, haloalkoxy, or cyano.
15. The compound of any of the previous embodiments 1-13 of Embodiment N wherein R⁶ and R⁷ are independently hydrogen, methyl, methoxy, fluoro, chloro, trifluoromethyl, trifluoromethoxy, or cyano. Within the groups in Embodiment 15, in one group of compound Ar is phenyl.
16. The compound of any of the previous embodiments 1-13 of Embodiment N wherein R⁷ is hydrogen and

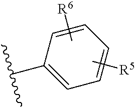

is a ring of formula:

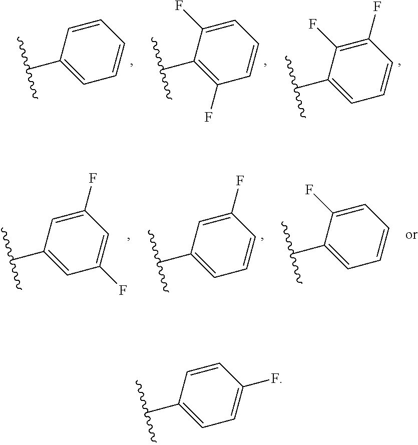

17. The compound of any of the previous embodiments 1-13 of Embodiment N wherein R⁷ is hydrogen and

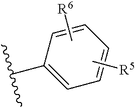

is a ring of formula:

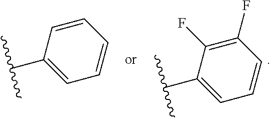 [SMILES: *c1ccccc1 ; *c1cccc(F)c1F]

Within the groups in embodiment 17, in one group of compounds

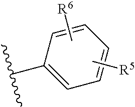

is phenyl. Within the groups in embodiment 17, in another group of compounds

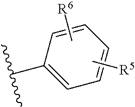

is a ring 2,3-difluorophenyl.
18. The compound of any of the previous embodiments 1-17 of Embodiment N wherein:

Z is bond or alkylene; EWG′ is

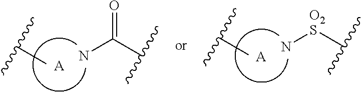

where ring A is heterocycloamino; R^{b} is cyano and R^{c} is isopropyl, tert-butyl, cyclopropyl, 1-methyl-1-methylaminoethyl, 1-methyl-1-dimethylaminoethyl, 1-methyl-1-aminoethyl, 1-methylaminocycloprop-1-ylene, 1-dimethylaminocycloprop-1-ylene, 1-ethoxy-1-methylethyl, —C(CH₃)₂morpholine-4-yl, 2-pyrrolidinyl, 3- or 4-piperidinyl, 1-methylpiperidin-4-yl, 1-methylpiperidin-3-yl, or 4-tetrahydropyranyl.
19. The compound of any of the previous embodiments 1-17 of Embodiment N wherein:

—Z-EWG′- is:

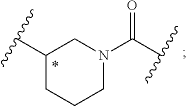 [SMILES: *C(=O)N1CCCC(*)C1]

R^{b} is cyano and R^{c} is isopropyl, tert-butyl, cyclopropyl, 1-methyl-1-methylaminoethyl, 1-methyl-1-dimethylaminoethyl, 1-methyl-1-aminoethyl, 1-methylaminocycloprop-1-ylene, 1-dimethylaminocycloprop-1-ylene, 1-ethoxy-1-methylethyl, —C(CH₃)₂morpholine-4-yl, 2-pyrrolidinyl, 3- or 4-piperidinyl, 1-methylpiperidin-4-yl, 1-methylpiperidin-3-yl, or 4-tetrahydropyranyl and the stereochemistry at *C is and the stereochemistry at *C is (R) or (S), preferably (R). Within the groups in embodiment 19, in one group of compounds R^{c} is isopropyl or tert-butyl. Within the groups in embodiment 19, in another group of compounds R^{c} is cyclopropyl. Within the groups in embodiment 19, in another group of compounds R^{c} is 1-methyl-1-methylaminoethyl, 1-methyl-1-dimethylaminoethyl, 1-methyl-1-aminoethyl, preferably 1-amino-1-methylethyl. Within the groups in embodiment 19, in another group of compounds R^{c} is 1-ethoxy-1-methylethyl. Within the groups in embodiment 19, in another group of compounds R^{c} is —C(CH₃)₂morpholine-4-yl. Within the groups in embodiment 19, in another group of compounds R^{c} is 2-pyrrolidinyl, 3- or 4-piperidinyl, 1-methylpiperidin-4-yl, 1-methylpiperidin-3-yl, or 4-tetrahydropyranyl.
20. The compound of any of the previous embodiments 1-17 of Embodiment N wherein:

—Z-EWG′- is:

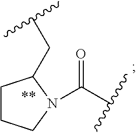 [SMILES: *CC1CCCN1C(*)=O]

R^{b} is cyano and R^{c} is isopropyl, tert-butyl, cyclopropyl, 1-methyl-1-methylaminoethyl, 1-methyl-1-dimethylaminoethyl, 1-methyl-1-aminoethyl, 1-methylaminocycloprop-1-ylene, 1-dimethylaminocycloprop-1-ylene, 1-ethoxy-1-methylethyl, —C(CH₃)₂morpholine-4-yl, 2-pyrrolidinyl, 3- or 4-piperidinyl, 1-methylpiperidin-4-yl, 1-methylpiperidin-3-yl, or 4-tetrahydropyranyl and the stereochemistry at **C is (R) or (S). Within the groups in embodiment 20, in one group of compounds R^{c} is isopropyl or tert-butyl. Within the groups in embodiment 20, in another group of compounds R^{c} is cyclopropyl. Within the groups in embodiment 20, in another group of compounds R^{c} is 1-methyl-1-methylaminoethyl, 1-methyl-1-dimethylaminoethyl, 1-methyl-1-aminoethyl, preferably 1-amino-1-methylethyl. Within the groups in embodiment 20, in another group of compounds R^{c} is 1-ethoxy-1-methylethyl. Within the groups in embodiment 20, in another group of compounds R^{c} is —C(CH₃)₂morpholine-4-yl. Within the groups in embodiment 20, in another group of compounds R^{c} is 2-pyrrolidinyl, 3- or 4-piperidinyl, 1-methylpiperidin-4-yl, 1-methylpiperidin-3-yl, or 4-tetrahydropyranyl.
21. The compound of any of the previous embodiments 1-5 and 8-11 of Embodiment N wherein:

R¹ is hydrogen, alkyl, hydroxy, alkoxy, halo, haloalkyl, or haloalkoxy;

R⁵ is —Z-(EWG′)-C(R^{b})═CHR^{e}; and

L is CONH or NHCONH.

22. The compound of any of the previous embodiments 1-5, 8-11 and 21 of Embodiment N where Z is bond or alkylene, EWG′ is —NR′CO— or —NR′SO₂—, preferably Z is bond or methylene and EWG′ is —NHCO—, and R^{c} is alkyl, substituted alkyl, haloalkoxy, cycloalkyl, cycloalkyleneNR^{d}R^{e} or 3 to 6 membered saturated monocyclic heterocyclyl containing one or two heteroatoms selected from N, O, or S and optionally substituted with one or two substituents selected from hydroxy, alkyl or fluoro. Within the groups in embodiment 22, in one group of compounds, R^{c} is alkyl. Within the groups in embodiment 22, in another group of compounds, R^{c} is cycloalkyl. Within the groups in embodiment 22, in another group of compounds, R^{c} is alkyl substituted with hydroxy, alkoxy, —NRR′ (where R is hydrogen, alkyl, hydroxyalkyl, or alkoxyalkyl, and R′ is hydrogen, alkyl, or cycloalkyl) or heterocyclyl (preferably heterocycloamino) optionally substituted with one or two groups independently selected from alkyl. Within the groups in embodiment 22, in another group of compounds, R^{c} is alkyl substituted with hydroxy or alkoxy. Within the groups in embodiment 22, in another group of compounds, R^{c} is alkyl substituted with —NRR′ (where R is hydrogen or and R′ is hydrogen or alkyl). Within the groups in embodiment 22, in another group of compounds, R^{c} is alkyl substituted with heterocycloamino optionally substituted with one or two groups independently selected from alkyl.
23. The compound of any of the previous embodiments 21 or 22 of Embodiment N wherein R⁶ and R⁷ are independently hydrogen, alkyl, alkoxy, halo, haloalkyl, haloalkoxy, or cyano.
24. The compound of any of the previous embodiments 21 or 22 of Embodiment N wherein R⁶ and R⁷ are independently hydrogen, methyl, methoxy, fluoro, chloro, trifluoromethyl, trifluoromethoxy, or cyano, preferably Ar is phenyl and R⁶ is hydrogen and R⁷ is trifluoromethyl.
25. The compound of any of the previous embodiments 21 or 22 of Embodiment N wherein

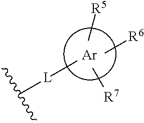

group is

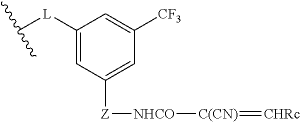

and is attached at the 4-position of the phenyl ring.

### General Synthetic Scheme

Compounds of this disclosure can be made by the methods depicted in the reaction schemes shown below.

The starting materials and reagents used in preparing these compounds are either available from commercial suppliers such as Aldrich Chemical Co., (Milwaukee, Wis.), Bachem (Torrance, Calif.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4th Edition) and Larock's Comprehensive Organic Transformations (VCH Publishers Inc., 1989). These schemes are merely illustrative of some methods by which the compounds of this disclosure can be synthesized, and various modifications to these schemes can be made and will be suggested to one skilled in the art having referred to this disclosure. The starting materials and the intermediates, and the final products of the reaction may be isolated and purified if desired using conventional techniques, including but not limited to filtration, distillation, crystallization, chromatography and the like. Such materials may be characterized using conventional means, including physical constants and spectral data.

Unless specified to the contrary, the reactions described herein take place at atmospheric pressure over a temperature range from about −78° C. to about 150° C., more preferably from about 0° C. to about 125° C. and most preferably at about room (or ambient) temperature, e.g., about 20° C.

Compounds of Formula (Ia) where Z¹ and Z² are carbon and Z³ is nitrogen R⁵ is hydrogen, alkyl, hydroxy, alkoxy, halo, haloalkyl, or haloalkoxy, and Ar, R¹, R³, R⁴, R⁶, R⁷, and L, are as defined above can be prepared as illustrated and described in Scheme A below.

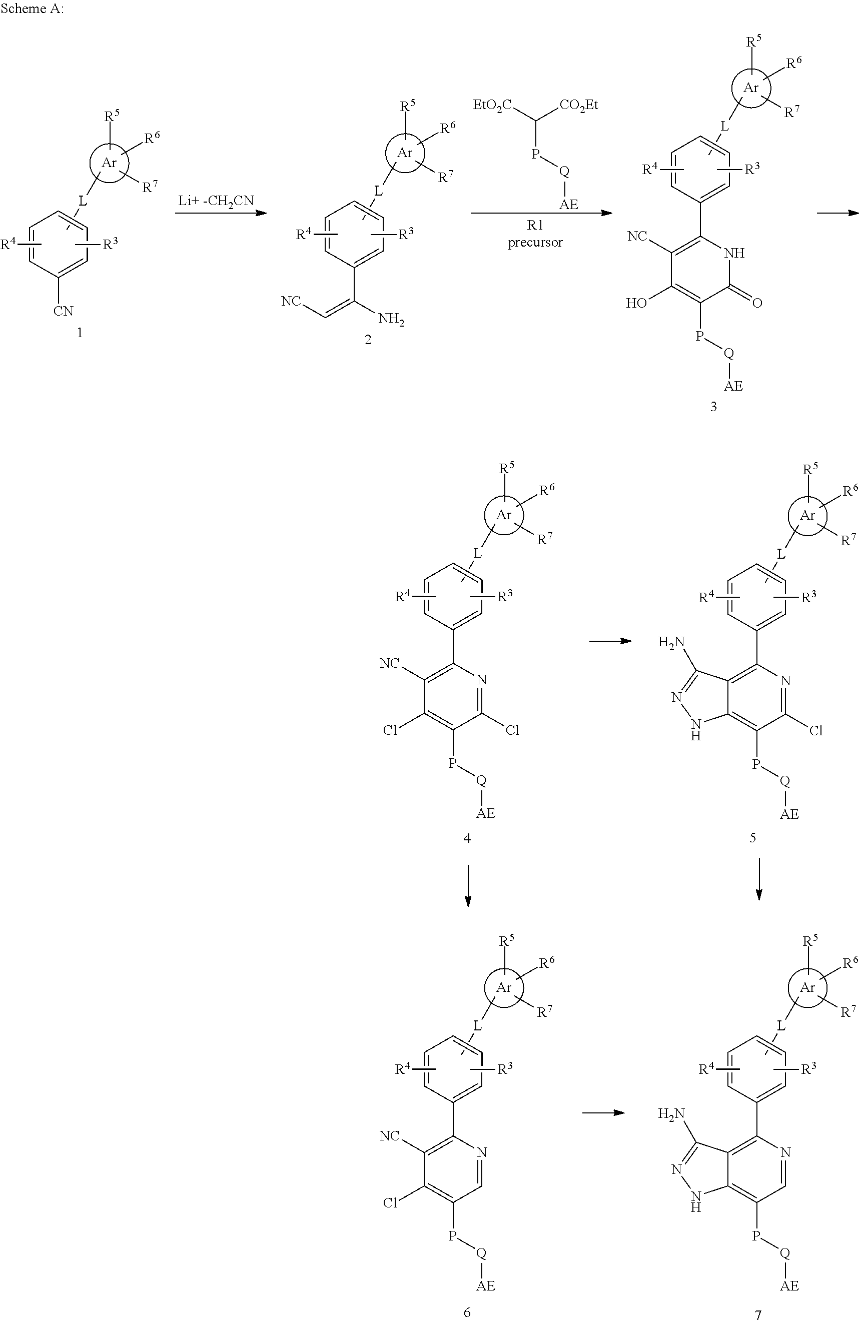

Scheme A illustrates two routes for construction of the 5-aza indazole ring system of formula (Ia), starting with the synthesis of an intermediate amino nitrile of formula (2). Deprotonation of acetonitrile with lithium di-isopropylamide (LDA) and addition to an appropriately functionalized aryl nitrile of formula (1) can provide an amino nitrile of formula (2) as a mixture of cis- and trans- isomers. Compounds of formula (2) can be reacted with an appropriately substituted diethylmalonate (R¹ precursor), under conventional or microwave heating, to provide a pyridone product (3) (Rivkin, A. Tetrahedron Letters 2006, 47 2395, and WO2011/019780). The R¹ precursor compound where P and Q are as defined above bears a suitable aldehyde equivalent (AE). The aldehyde equivalent (AE) is presented as a functional group that can be converted to the aldehyde in a simple transformation. Examples include: an acetal which can release the aldehyde under acidic conditions; a thioacetal which can release the aldehyde using mercuric or silver salts; incorporation of an alkene which can be oxidized with a mixture of osmium tetroxide and sodium periodate; cleavage of an alkene with ozone; deprotection of a primary alcohol and subsequent oxidation to the aldehyde. Several literature examples are known for generation of an aldehyde and selection of the appropriate aldehyde precursor is dependent on stability to other synthetic sequence transformations.

Upon heating with phosphoryl chloride, the pyridone (3) is converted to the chloropyridine (4). Cyclization of the chlorocyano pyridine (4) can be achieved by treatment with hydrazine under appropriate conditions in an alcoholic solvent such as methanol, ethanol, n-propanol, isopropanol, or n-butanol to give the 3-amino-5-aza-6-chloroindazole (5). Initial displacement of the chlorine is followed by ring closure under elevated temperature (typically 80-150° C.), or through subsequent exposure to an acid catalyst (i.e. hydrochloric acid, sulfuric acid, trifluoroacetic acid) at moderate temperature. Reduction of the chlorine proximal to nitrogen in formula (5) leads to the 3-amino-5-aza-indazole of general formula (7). Typical reducing agents include hydrazine followed by addition of copper (II) sulfate, Zn dust in the presence of acetic acid, or Red-Al in the presence of dichlorotitanocene.

Depending on reactivity of the chloropyridine (4) an alternative strategy effecting a selective reduction of the chlorine adjacent to the pyridine to provide (6) can be accomplished prior to ring formation with hydrazine.

Compounds of formula (7) are then converted to compounds of Formula (Ia) as shown below.

1. Method (a) describes the synthesis of compounds of formula (Ia) where R′ is —P-Q-CH═C(R^{b})(EWG) and P, Q, R^{b} and EWG are as defined above.

### Method (a):

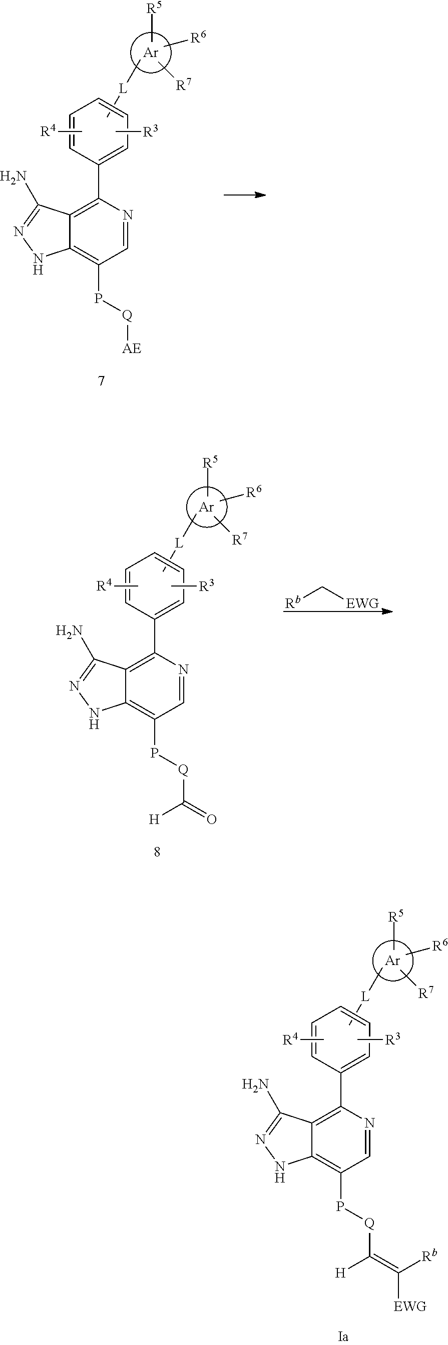

Conversion of AE in formula (7) to the aldehyde (8) can be achieved by any number of methods dependent on the form of AE as described above in Scheme A. Standard condensation reaction of formula (8) with R^{b}CH₂(EWG) in solvent (methanol, ethanol, THF) and base (piperidine or DBU) at temperatures ranging from 0° C. to 70° C. produce compounds of general Formula (Ia). Compounds of the formula R^{b}CH₂(EWG) are either commercially available or they can be prepared by methods well known in the art. For example, 2-cyano-N,N-dimethylacetamide and 2-trifluoromethyl-N,N-dimethyl acetamide are commercially available.

2. Substitution of precursors to R¹ in the synthesis of compounds of Formula (Ia) where R¹ is Z-(EWG′)-C(R^{b})═CHR^{c} where Z is a bond and EWG′ is an N-heterocycloamino-1-carbonyl illustrated in method (b) below.

### Method (b):

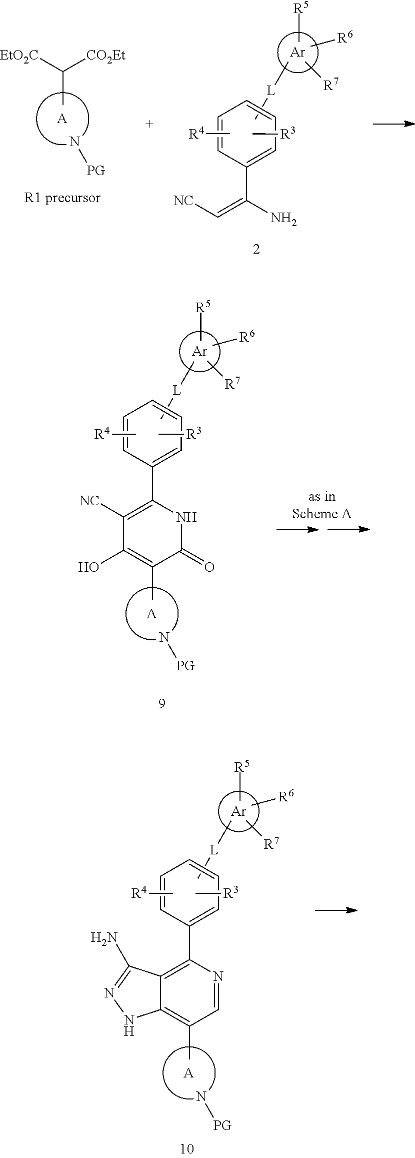

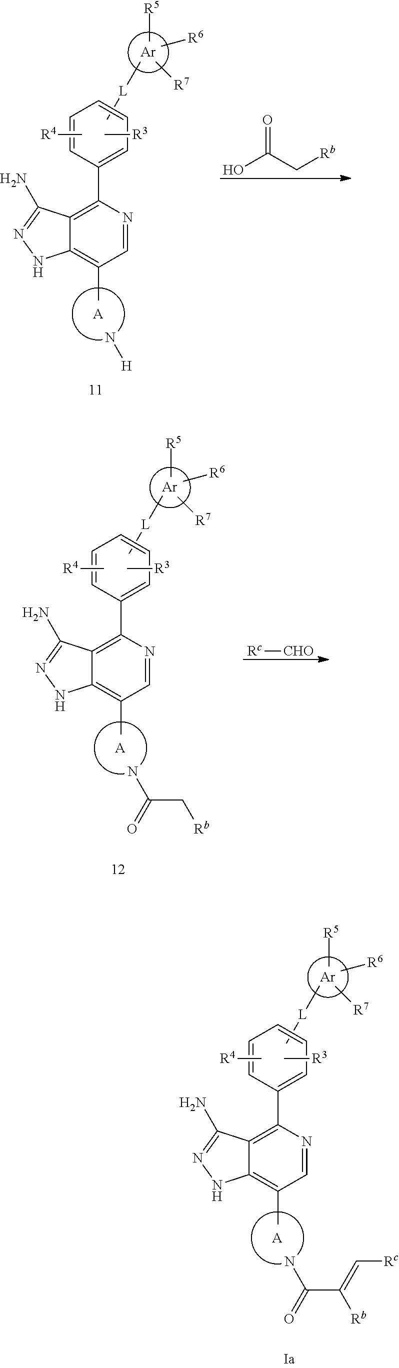

Treatment of a N-protected heterocycloamino R¹ precursor compound of a dialkyl malonate with a compound of formula (2) under conventional heating or microwave reaction conditions provides a compound of formula (9) where Ar, R³, R⁴, R⁵, R⁶, R⁷ and L are as defined above. Suitable nitrogen protecting groups (PG) include t-butyloxycarbonyl (BOC), carbobenzyloxy (Cbz), or 2-trimethylsilyl-ethoxymethyl (SEM). Chlorination, ring closure and de-chlorination can proceed by two alternative routes (as described in Scheme A) giving the 7-substituted N-protected heterocycloamino 5-aza indazole of formula (10).

Compounds of formula (10) can be converted to compounds of formula (11) by deprotection of the heterocycloamine. Deprotection can be effected using strong acid (TFA or HCL in the case of a Boc group, hydrogenolysis in the case of Cbz, or fluoride anion to remove the SEM), to provide the secondary amine of formula (11).

Coupling of compound of formula (11) with a compound of formula R^{b}CH₂CO₂H, such as 2-cyanoacetic acid or 2-trifluoromethylacetic acid, under standard amide coupling conditions such as carbon diimidazole (CDI) and the like, or an acid derivative thereof, provides a compound of formula (12). Subsequent condensation of this activated amide with aldehydes of formula R^{c}CHO, where R^{c} is as defined above, in an organic solvent such as ethanol and the like, at temperatures ranging from 0° C. to reflux, provides a compound of Formula Ia. It will be recognized by a person of ordinary skill in the art that the EWG′ moiety can be assembled at multiple points throughout the synthetic scheme and standard protecting group (PG) strategies can be employed as required.

Compounds of the Formula (Ib) where Z¹ and Z² are carbon and Z³ is nitrogen, R⁵ is hydrogen, alkyl, hydroxy, alkoxy, halo, haloalkyl, or haloalkoxy, R¹ is —NH-Q-CH═C(R^{b})(EWG) and Q, R^{b}, EWG, R¹, R³, R⁴, R⁵, R⁶, R⁷, L, and Ar are as defined above can be prepared as illustrated and described in Scheme B below.

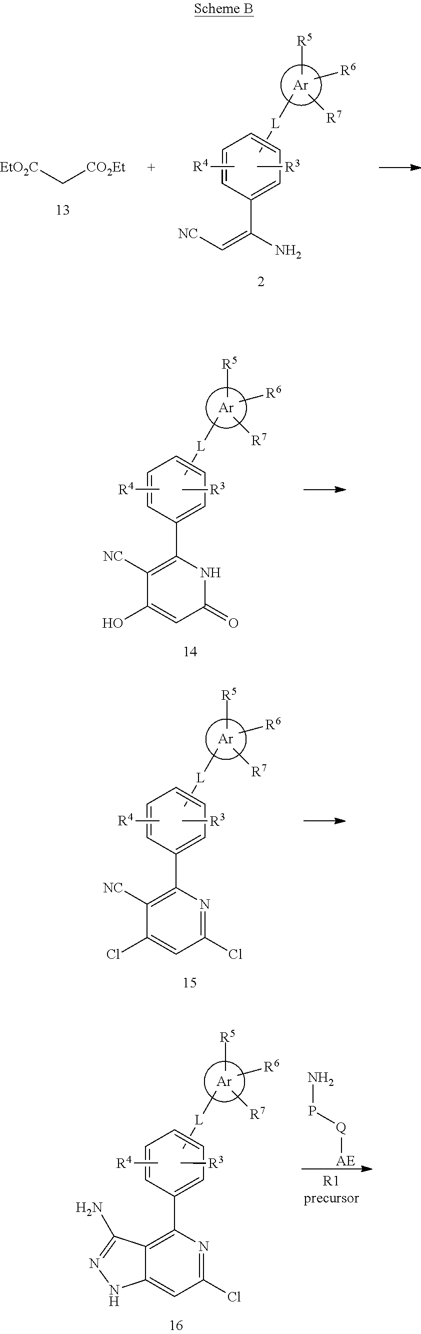

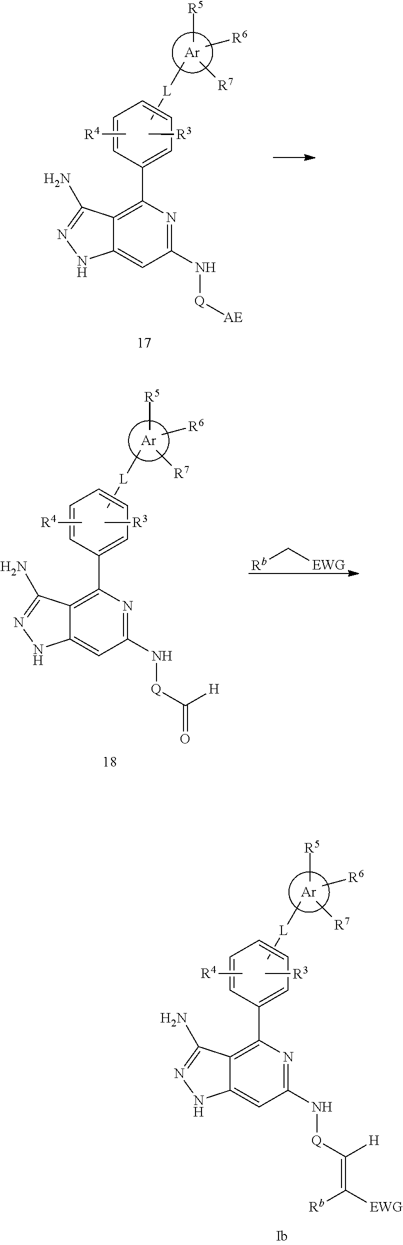

Cyclo-condensation of diethylmalonate (13) with a compound of formula (2) under conditions described in Scheme A produces a pyridone of formula (14). Chlorination to give formula (15) and ring formation with hydrazine proceeds as described in Scheme A above. Compounds of formula (17) can be formed by reacting the R¹ precursor amine with a compound of formula (16) in the presence of palladium catalyst (i.e. palladium acetate and the like), ligand (i.e. xantphos and the like) and base (i.e. cesium carbonate and the like) in a suitable solvent (i.e. THF and the like). The reaction proceeds in a temperature range of about 70° C. to about 180° C. and can take 10 minutes to 8 hours to complete.

Alternatively, a compound of formula (17) may be synthesized by reacting the R1 precursor amine with a compound of formula (16) in the presence of acid (i.e., HCl, TsOH and the like) in a suitable solvent (i.e. 2-propanol and the like). The reaction proceeds in a temperature range of about 70° C. to about 150° C. and can take up to 12 hours to complete. Alternatively, compounds of formula (17) can be prepared by reacting the R¹ precursor amine with a compound of formula (16) in the presence of a suitable solvent (i.e. DMF, DMSO) optionally in the presence of a suitable base (i.e. N,N-di-isopropylamine, sodium hydride, or 2,6-dimethylpyridine).

Alternatively, the compounds of formula (17) can be prepared by reacting the R¹ precursor amine with a compound of formula (16) neat under elevated temperature in a sealed tube. R¹ precursors where P is NH and Q are as defined above, and AE is aldehyde equivalent that acts as a precursor to an aldehyde, are either commercially available or they can be prepared by methods well known in the art.

Formation of the aldehyde (18) from a compound of formula (17) and subsequent condensation with a compound of formula R^{b}CH₂(EWG) under conditions described in Method (a) provides a compound of formula (Ib).

Compounds of the Formula (Ic) where Z¹ and Z³ are nitrogen and Z² is carbon R¹ is —NH-Q-CH═C(R^{b})(EWG) and Q, R^{b}, EWG, R¹, R³, R⁴, R⁵, R⁶, R⁷, L, and Ar are as defined above can be prepared as illustrated and described in Scheme C below.

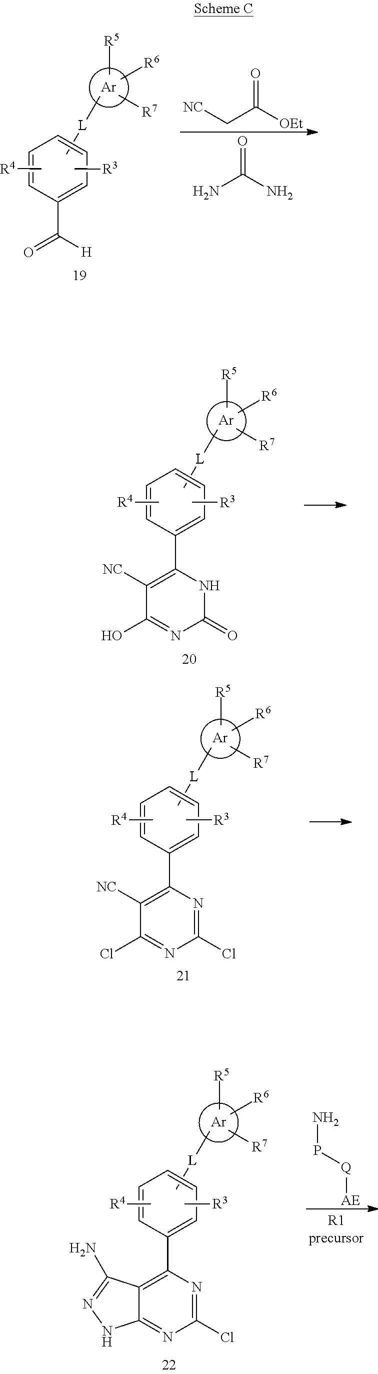

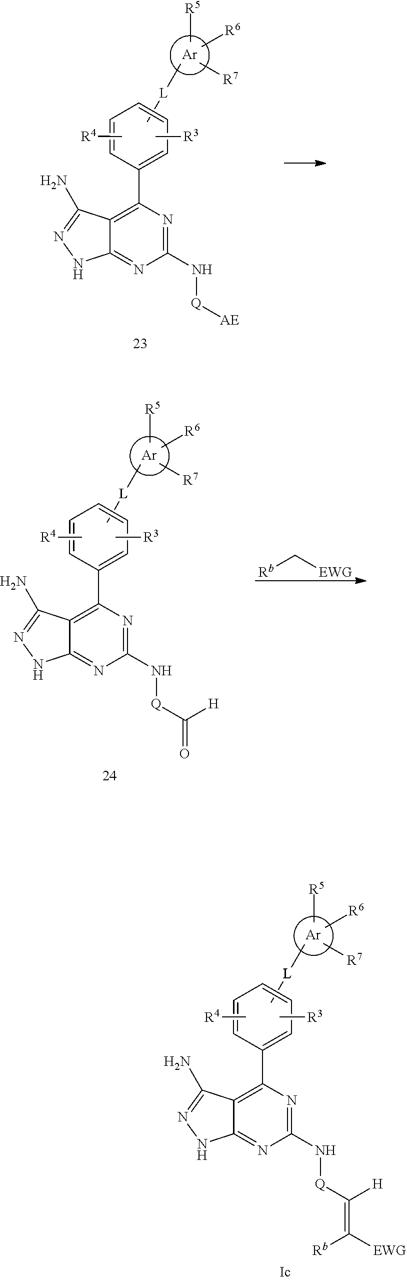

The pyrimidine of formula (20) can be formed by treatment of a benzaldehyde of formula (19) with ethyl cyanoacetate and urea in the presence of a base (potassium carbonate or the like) in a solvent (ethanol or the like) in a temperature range from about room temperature to 80° C. (see WO 2011/019780). After treating (20) with phosphoryl chloride at elevated temperature, the resulting dichloropyrimidine (21) can be converted to the 5,7-diazaindazole (Ic) using conditions described in Scheme A and method (a) above.

Compounds of the Formula (Id) where Z¹ and Z³ are carbon and Z² is nitrogen, R¹ is —P-Q-CH═C(R^{b})(EWG) and P, Q, R^{b}, EWG, R³, R⁴, R⁵, R⁶, R⁷, L, and Ar are as defined above can be prepared as illustrated and described in Scheme D below.

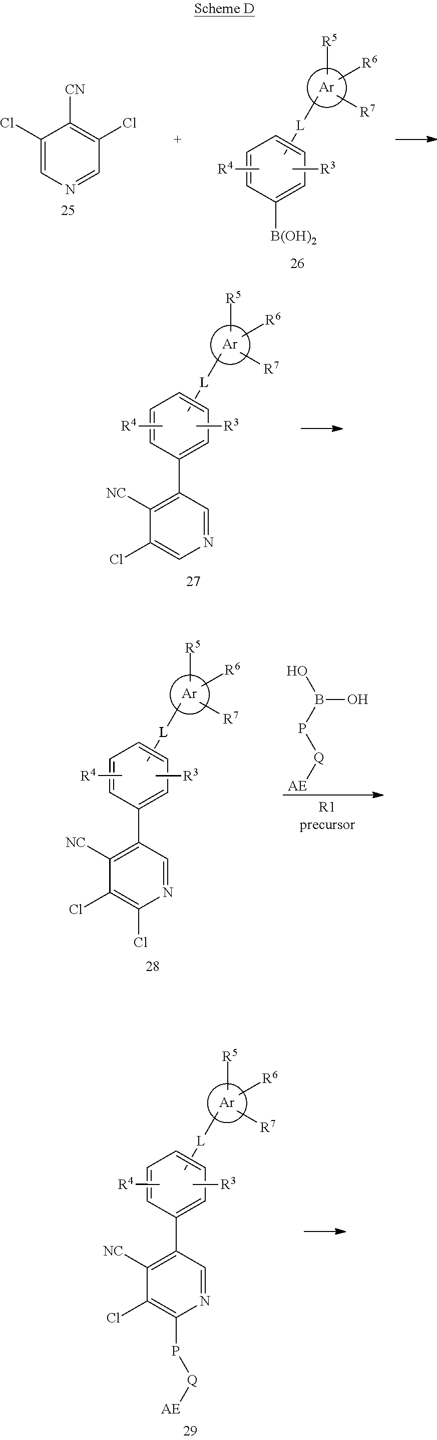

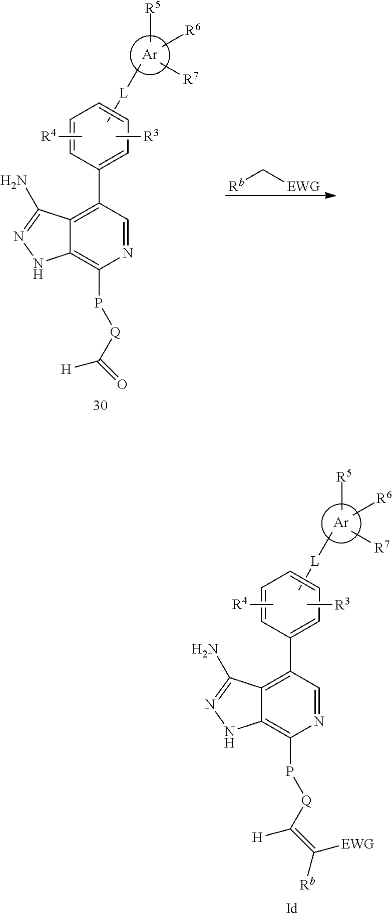

Conversion of 3,5-dichloroisonicotinonitrile (25) to compounds of formula (27) is accomplished under Suzuki conditions with an appropriately substituted aryl boronic acid (29) or aryl boronic ester thereof in the presence of a palladium catalyst and a base. Representative palladium catalysts include Pd(PPh₃)₄, Pd(o-tol₃P)₂Cl₂, PdCl₂(dppf), Pd(OAc) and PdCl₂(dppf).CH₂Cl₂. Examples of bases include triethylamine, sodium carbonate, cesium carbonate, and potassium carbonate, sodium ethylate, sodium hydroxide, potassium hydroxide, and the like. Solvents typically used in these reactions include DMF, DME, toluene, ethanol, water, and mixtures thereof. The reaction is typically conducted at temperatures between 60° C. and about 130° C. (optionally in a microwave for about 5 to 25 minutes) for about 4 to about 24 hours. The aryl boronic acids or aryl boronic esters are either commercially available or can be readily prepared by methods well known in the art.

Following conditions described in WO2011/019780, the dichloropyridine (28) can be formed from a compound of formula (27) first through N-oxide formation with m-CPBA and then subsequent treatment with phosphoryl chloride.

Suzuki cross coupling of a compound of formula (28) with a R¹ precursor compound depicted in Scheme D, where P and Q are as described above, and the R¹ precursor includes an aldehyde equivalent (AE) as described in Scheme A, provides a compound of formula (29). R¹ precursors of this type are either commercially available or they can be prepared by methods well known in the art. After formation of the aldehyde, (30) can be converted to the final compounds (Id) following conditions shown in Scheme A.

Compounds of the Formula (If) where Z¹, Z² and Z³ are carbon, L=NRC(O)NR′, R⁵ is —P-Q-CH═C(R^{b})(EWG) and P, Q, R^{b} and EWG and R¹, R³, R⁴, R⁶, R⁷, and Ar are as defined above, can be prepared as illustrated and described in Scheme E below.

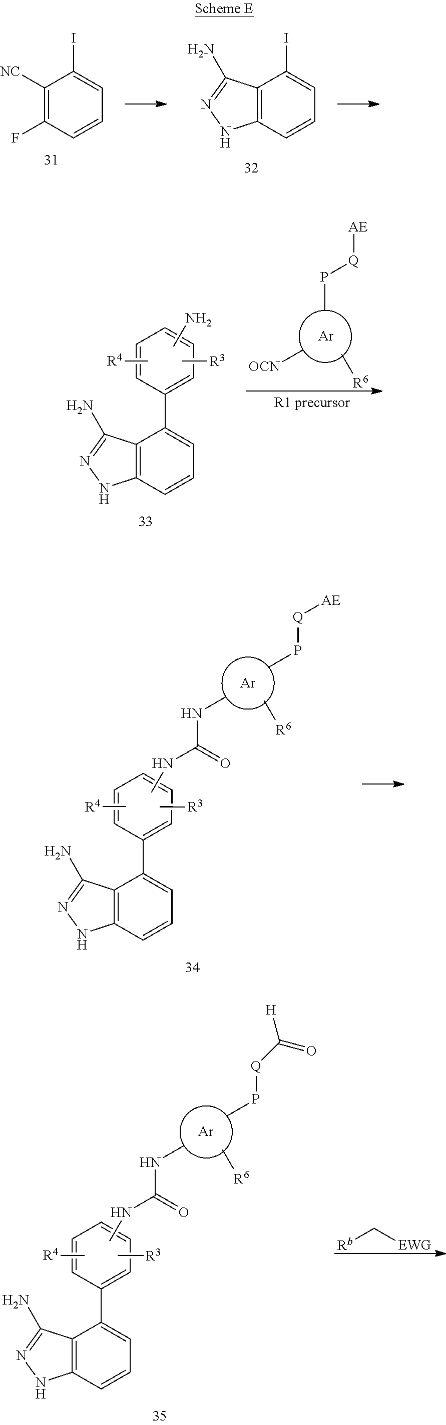

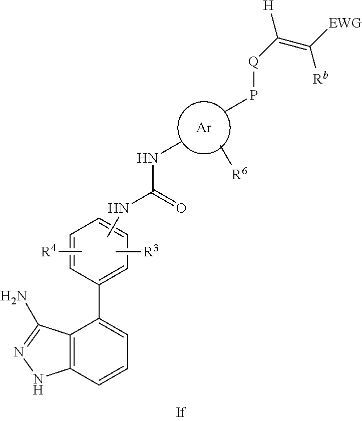

Treatment of commercially available 2-fluoro-6-iodobenzonitrile (31) with hydrazine results in the formation of 4-iodo-1H-indazole-3-amine (32) (see WO 2004/113304). Conversion of the iodo indazole (32) to compounds of formula (34) is accomplished using Suzuki conditions with an appropriately substituted anilino boronic acid or anilino boronic ester in the presence of a palladium catalyst and a base. The anilino boronic acids or anilino boronic esters are either commercially available or can be readily prepared by methods well known in the art.

Compounds of formula (33) can be reacted with an appropriately substituted isocyanate (shown as the R¹ precursor in Scheme E) to provide compounds of formula (34). Examples of solvents used in these reactions include THF, CH₂Cl₂, and MTBE. The reaction is typically conducted at a temperature of about 0° C. to about 25° C. for about 1 hour to about 14 hours. The R¹ precursor compound in Scheme E, where P and Q are as described above, and the R¹ precursor includes an aldehyde equivalent (AE) as described in Scheme A, are either commercially available or they can be prepared by methods well known in the art. After formation of the aldehyde, (35) can be converted to the final compounds (If) following conditions indicated in Scheme A.

Compounds of the Formula (If) where Z¹, Z² and Z³ are carbon, L=NHC(O)NH, and R¹, R³, R⁴, R⁶, R⁷, and Ar are as defined above, and when R⁵ is Z-(EWG′)-C(R^{b})═CHR^{c} where Z is a bond and EWG′ is an amide formed from a heterocycloamino is illustrated in Scheme F below. The EWG moiety can be assembled at multiple points in the synthetic scheme.

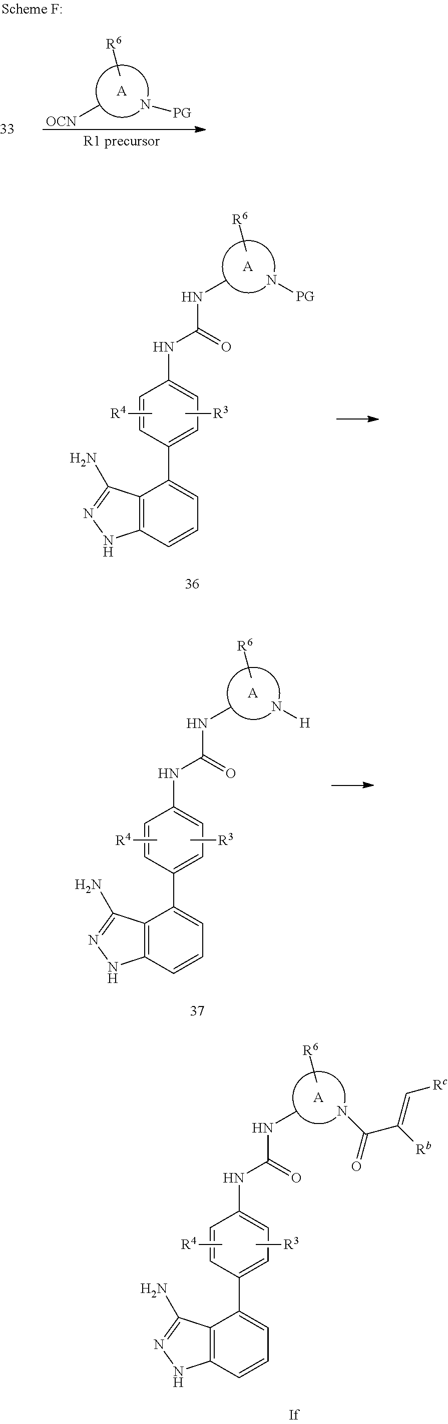

Upon reaction of the R¹ precursor isocyanate with a compound of formula (33), a urea compound of formula (35) is formed. Further conversion to compounds of formula (If) is accomplished using procedures outlined in Method (a) above. The R¹ precursor isocyanate in Scheme F, where P and Q are as described above, and the R¹ precursor includes a N-protected heterocycloamino (A), are either commercially available or they can be prepared by methods well known in the art.

Compounds of the Formula (If) where Z¹, Z² and Z³ are carbon and L=NHC(O)NH, and R¹, R³, R⁴, R⁶, R⁷, and Ar are as defined above, and R⁵ is —Z-(EWG′)-C(R^{b})═CH(R^{c}) can be prepared as illustrated and described in Scheme G below. For the R1 precursor isocyanate in Scheme G, P and Q are a bond and Ar is EWG in nature (i.e. pyridine, pyrimidine and the like) with a methyl group adjacent to the ring nitrogen.

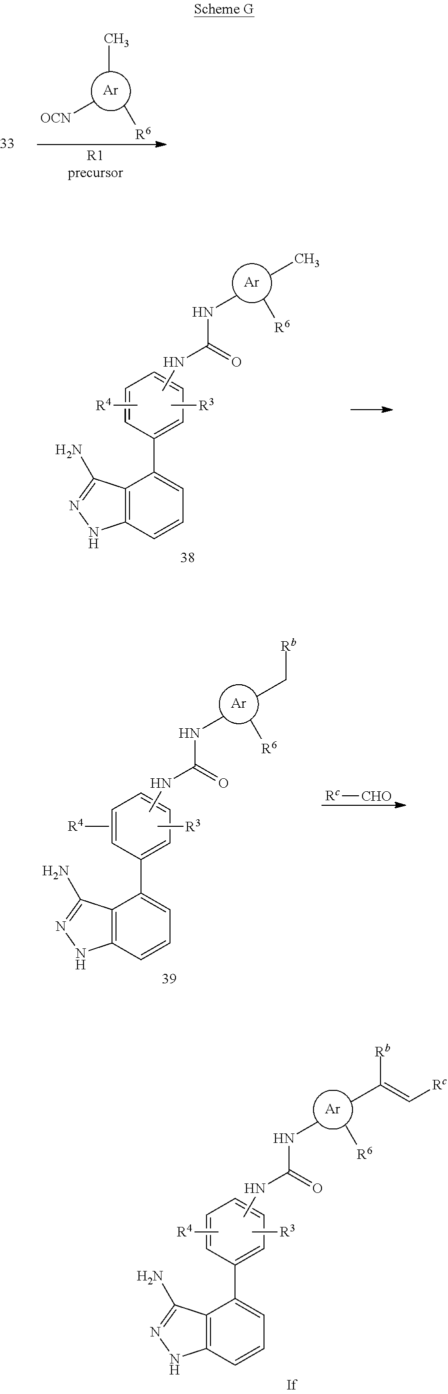

Upon reaction of the R¹ precursor isocyanate with a compound of formula (33), a urea compound of formula (35) is formed. Activation of the methyl group adjacent to the aromatic ring nitrogen can occur by a variety of methods. One method could utilize oxidation of the ring nitrogen with mCPBA using dichloromethane as solvent at room temperature to reflux, and subsequent treatment with acetic anhydride to give the methyl acetate. Alternatively, the methyl group could be halogenated with NBS in the presence of AIBN to provide the bromomethyl compound. The bromine or the acetate can then be displaced with an R^{b} equivalent (for example CN using KCN with DMSO as solvent) to provide a compound of formula (39). Treatment of a compound of formula (39) with a simple aldehyde of formula R^{c}—CHO (for example, pivaloyl aldehyde or cyclopropyl aldehyde) in the presence of a base (for example piperidine or DBU) and a catalyst (such as acetic acid or the like) in an alcoholic solvent (ethanol and the like) at temperatures ranging from about 25° C. to 80° C., will provide compounds of the formula (If).

Compounds of Formula (Ig) where L, Z, A, R^{b}. R^{c}. R³, R⁴, R⁵, R⁶, R⁷, and Ar are as defined above, can be prepared as illustrated and described in Scheme H below.

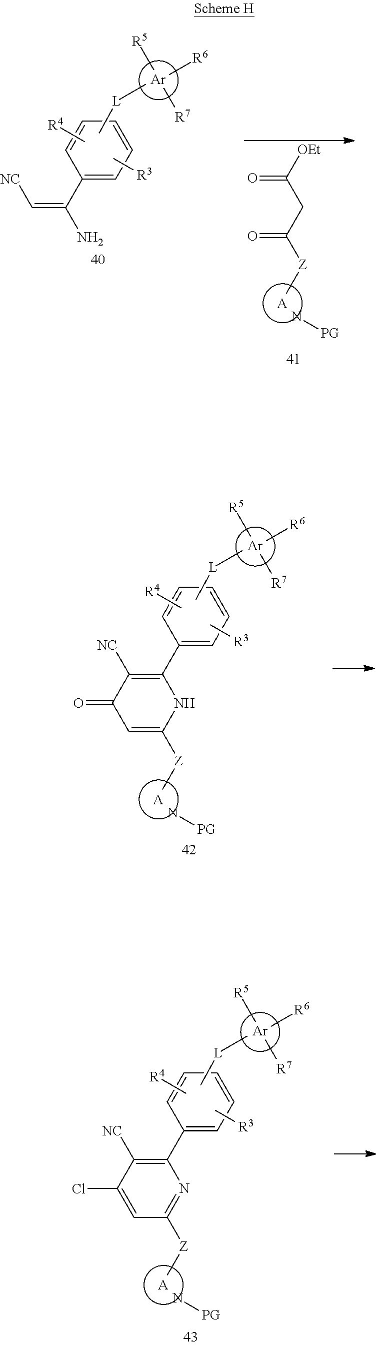

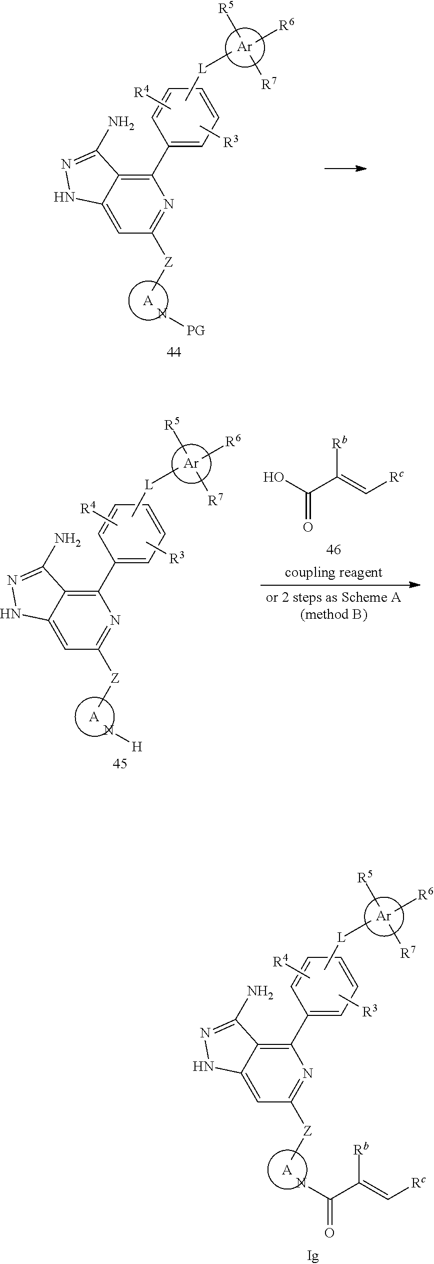

Compounds of formula (40) and formula (41) can be heated in DMF or another suitable solvent to afford compounds of formula (42). Chlorination to afford compounds (43) can be accomplished by the action of POCl₃. Following chlorination, heating of compounds of formula (43) with hydrazine in a solution such as DMF, DMA, BuOH, PrOH, or other solvent affords compounds of formula (44). Deprotection of a protecting group through conditions known in the art affords compounds (45). Coupling of compounds (45) and acids of formula (46) with PyBrOP or another amino acid coupling reagent known in the art, yields compounds (Ig). Alternatively, compounds of formula (Ig) can be prepared first by coupling cyanoacetic acid, followed by condensation with an appropriate aldehyde as described in Method (B) above.

### Utility

The compounds of Formula (I′) or (I) are kinase inhibitors, in particular BTK and hence are useful in the treatment of autoimmune disease, e.g., inflammatory bowel disease, arthritis, lupus, rheumatoid arthritis, psoriatic arthritis, osteoarthritis, Still's disease, juvenile arthritis, diabetes, myasthenia gravis, Hashimoto's thyroiditis, Ord's thyroiditis, Graves' disease, Sjogren's syndrome, multiple sclerosis, Guillain-Barre syndrome, acute disseminated encephalomyelitis, Addison's disease, opsoclonus-myoclonus syndrome, ankylosing spondylitisis, antiphospholipid antibody syndrome, aplastic anemia, autoimmune hepatitis, coeliac disease, Goodpasture's syndrome, idiopathic thrombocytopenic purpura, optic neuritis, scleroderma, primary biliary cirrhosis, Reiter's syndrome, Takayasu's arteritis, temporal arteritis, warm autoimmune hemolytic anemia, Wegener's granulomatosis, psoriasis, alopecia universalis, Behcet's disease, chronic fatigue, dysautonomia, endometriosis, interstitial cystitis, neuromyotonia, scleroderma, or vulvodynia.

The compounds of Formula (I′) or (I) are also useful in the treatment of In another embodiment of this aspect, the patient in need is suffering from a heteroimmune condition or disease, e.g., graft versus host disease, transplantation, transfusion, anaphylaxis, allergy, type I hypersensitivity, allergic conjunctivitis, allergic rhinitis, or atopic dermatitis.

In another embodiment of this aspect, the patient in need is suffering from an inflammatory disease, e.g., asthma, appendicitis, blepharitis, bronchiolitis, bronchitis, bursitis, cervicitis, cholangitis, cholecystitis, colitis, conjunctivitis, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, hepatitis, hidradenitis suppurativa, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, proctitis, prostatitis, pyelonephritis, rhinitis, salpingitis, sinusitis, stomatitis, synovitis, tendonitis, tonsillitis, uveitis, vaginitis, vasculitis, or vulvitis.

In another embodiment of this aspect, the patient is suffering from inflammatory skin disease which includes, by way of example, dermatitis, contact dermatitis, eczema, urticaria, rosacea, and scarring psoriatic lesions in the skin, joints, or other tissues or organs.

In yet another embodiment of this aspect, the subject in need is suffering from a cancer. In one embodiment, the cancer is a B-cell proliferative disorder, e.g., diffuse large B cell lymphoma, follicular lymphoma, chronic lymphocytic lymphoma, chronic lymphocytic leukemia, B-cell prolymphocytic leukemia, lymphoplamascytic lymphoma/Waldenstrom macroglobulinemia, splenic marginal zone lymphoma, plasma cell myeloma, plasmacytoma, extranodal marginal zone B cell lymphoma, nodal marginal zone B cell lymphoma, mantle cell lymphoma, mediastinal (thymic) large B cell lymphoma, intravascular large B cell lymphoma, primary effusion lymphoma, burkitt lymphoma/leukemia, or lymphomatoid granulomatosis. In some embodiments, the compound of Formula (I′) or (I) is administered in combination with another an anti-cancer agent e.g., the anti-cancer agent is an inhibitor of mitogen-activated protein kinase signaling, e.g., U0126, PD98059, PD184352, PD0325901, ARRY-142886, SB239063, SP600125, BAY 43-9006, wortmannin, or LY294002.

In yet another embodiment, the patient in need is suffering from a thromboembolic disorder, e.g., myocardial infarct, angina pectoris, reocclusion after angioplasty, restenosis after angioplasty, reocclusion after aortocoronary bypass, restenosis after aortocoronary bypass, stroke, transitory ischemia, a peripheral arterial occlusive disorder, pulmonary embolism, or deep venous thrombosis.

In a fourth aspect, the disclosure is directed to use of compound of Formula (I′) or (I) (and any embodiments thereof described herein) for use as a medicament. In one embodiment, the use of compound of Formula (I′) or (I) is for treating inflammatory disease or proliferative diseases.

In a fifth aspect is the use of a compound of Formula (I′) or (I) in the manufacture of a medicament for treating an inflammatory disease in a patient in which the activity of BTK or other tyrosine kinases contributes to the pathology and/or symptoms of the disease. In one embodiment of this aspect, the tyrosine kinase protein is BTK. In another embodiment of this aspect, the inflammatory disease is respiratory, cardiovascular, or proliferative diseases. In any of the aforementioned aspects involving the treatment of proliferative disorders, including cancer, are further embodiments comprising administering the compound of Formula (I′) or (I) in combination with at least one additional agent selected from the group consisting of alemtuzumab, arsenic trioxide, asparaginase (pegylated or non-), bevacizumab, cetuximab, platinum-based compounds such as cisplatin, cladribine, daunorubicin/doxorubicin/idarubicin, irinotecan, fludarabine, 5-fluorouracil, gemtuzamab, methotrexate, Paclitaxel™, taxol, temozolomide, thioguanine, or classes of drugs including hormones (an antiestrogen, an antiandrogen, or gonadotropin releasing hormone analogues, interferons such as alpha interferon, nitrogen mustards such as busulfan or melphalan or mechlorethamine, retinoids such as tretinoin, topoisomerase inhibitors such as irinotecan or topotecan, tyrosine kinase inhibitors such as gefinitinib or imatinib, or agents to treat signs or symptoms induced by such therapy including allopurinol, filgrastim, granisetron/ondansetron/palonosetron, dronabinol. When combination therapy is used, the agents can be administered simultaneously or sequentially.

### Testing

The kinase inhibitory activity of the compounds, including BTK, of the present disclosure can be tested using the in vitro and in vivo assays described in Biological Examples 1-7 below. The ability of the compounds of the disclosure to form a reversible covalent bond with a cysteine residue of a kinase, preferably Cys481 of BTK (UniprotKB Sequence ID Q06187), can be determined by the assays described in Example 8-11 below

### Administration and Pharmaceutical Composition

In general, the compounds of this disclosure will be administered in a therapeutically effective amount by any of the accepted modes of administration for agents that serve similar utilities. Therapeutically effective amounts of compounds of Formula (I′) or (I) may range from about 0.01 to about 500 mg per kg patient body weight per day, which can be administered in single or multiple doses. Preferably, the dosage level will be about 0.1 to about 250 mg/kg per day; more preferably about 0.5 to about 100 mg/kg per day. A suitable dosage level may be about 0.01 to about 250 mg/kg per day, about 0.05 to about 100 mg/kg per day, or about 0.1 to about 50 mg/kg per day. Within this range the dosage can be about 0.05 to about 0.5, about 0.5 to about 5 or about 5 to about 50 mg/kg per day. For oral administration, the compositions are preferably provided in the form of tablets containing about 1.0 to about 1000 milligrams of the active ingredient, particularly about 1.0, 5.0, 10, 15, 20, 25, 50, 75, 100, 150, 200, 250, 300, 400, 500, 600, 750, 800, 900, and 1000 milligrams of the active ingredient. The actual amount of the compound of this disclosure, i.e., the active ingredient, will depend upon numerous factors such as the severity of the disease to be treated, the age and relative health of the subject, the potency of the compound being utilized, the route and form of administration, and other factors.

In general, compounds of this disclosure will be administered as pharmaceutical compositions by any one of the following routes: oral, systemic (e.g., transdermal, intranasal or by suppository), or parenteral (e.g., intramuscular, intravenous or subcutaneous) administration. The preferred manner of administration is oral using a convenient daily dosage regimen, which can be adjusted according to the degree of affliction. Compositions can take the form of tablets, pills, capsules, semisolids, powders, sustained release formulations, solutions, suspensions, elixirs, aerosols, or any other appropriate compositions.

The choice of formulation depends on various factors such as the mode of drug administration (e.g., for oral administration, formulations in the form of tablets, pills or capsules are preferred) and the bioavailability of the drug substance. Recently, pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of in general, a compound of formula (I′) or (I) in combination with at least one pharmaceutically acceptable excipient. Acceptable excipients are non-toxic, aid administration, and do not adversely affect the therapeutic benefit of the compound of formula (I′) or (I). Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

Compressed gases may be used to disperse a compound of this disclosure in aerosol form. Inert gases suitable for this purpose are nitrogen, carbon dioxide, etc.

Other suitable pharmaceutical excipients and their formulations are described in Remington's Pharmaceutical Sciences, edited by E. W. Martin (Mack Publishing Company, 20th ed., 2000).

The level of the compound in a formulation can vary within the full range employed by those skilled in the art. Typically, the formulation will contain, on a weight percent (wt %) basis, from about 0.01-99.99 wt % of a compound of formula (I′) or (I) based on the total formulation, with the balance being one or more suitable pharmaceutical excipients. Preferably, the compound is present at a level of about 1-80 wt %.

The compounds of the present disclosure may be used in combination with one or more other drugs in the treatment of diseases or conditions for which compounds of the present disclosure or the other drugs may have utility, where the combination of the drugs together are safer or more effective than either drug alone. Such other drug(s) may be administered, by a route and in an amount commonly used therefore, contemporaneously or sequentially with a compound of the present disclosure. When a compound of the present disclosure is used contemporaneously with one or more other drugs, a pharmaceutical composition in unit dosage form containing such other drugs and the compound of the present disclosure is preferred. However, the combination therapy may also include therapies in which the compound of the present disclosure and one or more other drugs are administered on different overlapping schedules. It is also contemplated that when used in combination with one or more other active ingredients, the compounds of the present disclosure and the other active ingredients may be used in lower doses than when each is used singly.

Accordingly, the pharmaceutical compositions of the present disclosure also include those that contain one or more other active ingredients, in addition to a compound of the present disclosure.

The above combinations include combinations of a compound of the present disclosure not only with one other active compound, but also with two or more other active compounds. Likewise, compounds of the present disclosure may be used in combination with other drugs that are used in the prevention, treatment, control, amelioration, or reduction of risk of the diseases or conditions for which compounds of the present disclosure are useful. Such other drugs may be administered, by a route and in an amount commonly used therefore, contemporaneously or sequentially with a compound of the present disclosure. When a compound of the present disclosure is used contemporaneously with one or more other drugs, a pharmaceutical composition containing such other drugs in addition to the compound of the present disclosure is preferred. Accordingly, the pharmaceutical compositions of the present disclosure also include those that also contain one or more other active ingredients, in addition to a compound of the present disclosure. The weight ratio of the compound of the present disclosure to the second active ingredient may be varied and will depend upon the effective dose of each ingredient. Generally, an effective dose of each will be used.

Where the subject is suffering from or at risk of suffering from an autoimmune disease, an inflammatory disease, or an allergy disease, a compound of Formula (I′) or (I) can be used in with one or more of the following therapeutic agents in any combination: immunosuppressants (e.g., tacrolimus, cyclosporin, rapamicin, methotrexate, cyclophosphamide, azathioprine, mercaptopurine, mycophenolate, or FTY720), glucocorticoids (e.g., prednisone, cortisone acetate, prednisolone, methylprednisolone, dexamethasone, betamethasone, triamcinolone, beclometasone, fludrocortisone acetate, deoxycorticosterone acetate, aldosterone), non-steroidal anti-inflammatory drugs (e.g., salicylates, arylalkanoic acids, 2-arylpropionic acids, N-arylanthranilic acids, oxicams, coxibs, or sulphonanilides), Cox-2-specific inhibitors (e.g., valdecoxib, celecoxib, or rofecoxib), leflunomide, gold thioglucose, gold thiomalate, aurofin, sulfasalazine, hydroxychloroquinine, minocycline, TNF-.alpha. binding proteins (e.g., infliximab, etanercept, or adalimumab), abatacept, anakinra, interferon-.beta., interferon-.gamma., interleukin-2, allergy vaccines, antihistamines, antileukotrienes, beta-agonists, theophylline, or anticholinergics.

Where the subject is suffering from or at risk of suffering from a B-cell proliferative disorder (e.g., plasma cell myeloma), the subject can be treated with a compound of Formula (I′) or (I) in any combination with one or more other anti-cancer agents. In some embodiments, one or more of the anti-cancer agents are proapoptotic agents. Examples of anti-cancer agents include, but are not limited to, any of the following: gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec™), geldanamycin, 17-N-Allylamino-17-Demethoxygeldanamycin (17-AAG), flavopiridol, LY294002, bortezomib, trastuzumab, BAY 11-7082, PKC412, or PD184352, Taxol™, also referred to as “paclitaxel”, which is a well-known anti-cancer drug which acts by enhancing and stabilizing microtubule formation, and analogs of Taxol™., such as Taxotere™. Compounds that have the basic taxane skeleton as a common structure feature, have also been shown to have the ability to arrest cells in the G2-M phases due to stabilized microtubules and may be useful for treating cancer in combination with the compounds described herein.

Further examples of anti-cancer agents for use in combination with a compound of Formula (I′) or (I) include inhibitors of mitogen-activated protein kinase signaling, e.g., U0126, PD98059, PD184352, PD0325901, ARRY-142886, SB239063, SP600125, BAY 43-9006, wortmannin, or LY294002; Syk inhibitors; mTOR inhibitors; and antibodies (e.g., rituxan).

Other anti-cancer agents that can be employed in combination with a compound of Formula (I′) or (I) include Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Other anti-cancer agents that can be employed in combination with a compound of Formula (I′) or (I) include: 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflomithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; fmasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; R.sub.11 retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

Yet other anticancer agents that can be employed in combination with a compound of Formula (I′) or (I) include alkylating agents, antimetabolites, natural products, or hormones, e.g., nitrogen mustards (e.g., mechloroethamine, cyclophosphamide, chlorambucil, etc.), alkyl sulfonates (e.g., busulfan), nitrosoureas (e.g., carmustine, lomusitne, etc.), or triazenes (decarbazine, etc.). Examples of antimetabolites include but are not limited to folic acid analog (e.g., methotrexate), or pyrimidine analogs (e.g., Cytarabine), purine analogs (e.g., mercaptopurine, thioguanine, pentostatin).

Examples of natural products useful in combination with a compound of Formula (I′) or (I) include but are not limited to vinca alkaloids (e.g., vinblastin, vincristine), epipodophyllotoxins (e.g., etoposide), antibiotics (e.g., daunorubicin, doxorubicin, bleomycin), enzymes (e.g., L-asparaginase), or biological response modifiers (e.g., interferon alpha).

Examples of alkylating agents that can be employed in combination a compound of Formula (I′) or (I) include, but are not limited to, nitrogen mustards (e.g., mechloroethamine, cyclophosphamide, chlorambucil, melphalan, etc.), ethylenimine and methylmelamines (e.g., hexamethlymelamine, thiotepa), alkyl sulfonates (e.g., busulfan), nitrosoureas (e.g., carmustine, lomusitne, semustine, streptozocin, etc.), or triazenes (decarbazine, etc.). Examples of antimetabolites include, but are not limited to folic acid analog (e.g., methotrexate), or pyrimidine analogs (e.g., fluorouracil, floxuridine, Cytarabine), purine analogs (e.g., mercaptopurine, thioguanine, pentostatin.

Examples of hormones and antagonists useful in combination a compound of Formula (I′) or (I) include, but are not limited to, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), gonadotropin releasing hormone analog (e.g., leuprolide). Other agents that can be used in the methods and compositions described herein for the treatment or prevention of cancer include platinum coordination complexes (e.g., cisplatin, carboblatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide).

Examples of anti-cancer agents which act by arresting cells in the G2-M phases due to stabilized microtubules and which can be used in combination with an irreversible BTK inhibitor compound include without limitation the following marketed drugs and drugs in development: Erbulozole (also known as R-55104), Dolastatin 10 (also known as DLS-10 and NSC-376128), Mivobulin isethionate (also known as CI-980), Vincristine, NSC-639829, Discodermolide (also known as NVP-XX-A-296), ABT-751 (Abbott, also known as E-7010), Altorhyrtins (such as Altorhyrtin A and Altorhyrtin C), Spongistatins (such as Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (also known as LU-103793 and NSC-D-669356), Epothilones (such as Epothilone A, Epothilone B, Epothilone C (also known as desoxyepothilone A or dEpoA), Epothilone D (also referred to as KOS-862, dEpoB, and desoxyepothilone B), Epothilone E, Epothilone F, Epothilone B N-oxide, Epothilone A N-oxide, 16-aza-epothilone B, 21-aminoepothilone B (also known as BMS-310705), 21-hydroxyepothilone D (also known as Desoxyepothilone F and dEpoF), 26-fluoroepothilone), Auristatin PE (also known as NSC-654663), Soblidotin (also known as TZT-1027), LS-4559-P (Pharmacia, also known as LS-4577), LS-4578 (Pharmacia, also known as LS-477-P), LS-4477 (Pharmacia), LS-4559 (Pharmacia), RPR-112378 (Aventis), Vincristine sulfate, DZ-3358 (Daiichi), FR-182877 (Fujisawa, also known as WS-9885B), GS-164 (Takeda), GS-198 (Takeda), KAR-2 (Hungarian Academy of Sciences), BSF-223651 (BASF, also known as ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (also known as LY-355703), AC-7739 (Ajinomoto, also known as AVE-8063A and CS-39.HCl), AC-7700 (Ajinomoto, also known as AVE-8062, AVE-8062A, CS-39-L-Ser.HCl, and RPR-258062A), Vitilevuamide, Tubulysin A, Canadensol, Centaureidin (also known as NSC-106969), T-138067 (Tularik, also known as T-67, TL-138067 and TI-138067), COBRA-1 (Parker Hughes Institute, also known as DDE-261 and WHI-261), H10 (Kansas State University), H16 (Kansas State University), Oncocidin A1 (also known as BTO-956 and DIME), DDE-313 (Parker Hughes Institute), Fijianolide B. Laulimalide, SPA-2 (Parker Hughes Institute), SPA-1 (Parker Hughes Institute, also known as SPIKET-P), 3-IAABU (Cytoskeleton/Mt. Sinai School of Medicine, also known as MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, also known as MF-191), TMPN (Arizona State University), Vanadocene acetylacetonate, T-138026 (Tularik), Monsatrol, Inanocine (also known as NSC-698666), 3-1AABE (Cytoskeleton/Mt. Sinai School of Medicine), A-204197 (Abbott), T-607 (Tuiarik, also known as T-900607), RPR-115781 (Aventis), Eleutherobins (such as Desmethyleleutherobin, Desaetyleleutherobin, Isoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (also known as NSCL-96F037), D-68838 (Asta Medica), D-68836 (Asta Medica), Myoseverin B, D-43411 (Zentaris, also known as D-81862), A-289099 (Abbott), A-318315 (Abbott), HTI-286 (also known as SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi).

Where the subject is suffering from or at risk of suffering from a thromboembolic disorder (e.g., stroke), the subject can be treated with a compound of Formula (I′) or (I) in any combination with one or more other anti-thromboembolic agents. Examples of anti-thromboembolic agents include, but are not limited any of the following: thrombolytic agents (e.g., alteplase anistreplase, streptokinase, urokinase, or tissue plasminogen activator), heparin, tinzaparin, warfarin, dabigatran (e.g., dabigatran etexilate), factor Xa inhibitors (e.g., fondaparinux, draparinux, rivaroxaban, DX-9065a, otamixaban, LY517717, or YM150), ticlopidine, clopidogrel, CS-747 (prasugrel, LY640315), ximelagatran, or BIBR 1048.

### EXAMPLES

The following preparations of compounds of Formula (I′) or (I) and intermediates (References) are given to enable those skilled in the art to more clearly understand and to practice the present disclosure. They should not be considered as limiting the scope of the disclosure, but merely as being illustrative and representative thereof. The [symbol] line in the compounds below denotes that the compounds are isolated as an undefined mixture of (E) and (Z) isomers.

### Synthetic Examples

### Reference 1

### Synthesis of 5-(3-(1,3-dioxolan-2-yl)phenyl)-4-hydroxy-6-oxo-2-(4-phenoxyphenyl)-1,6-dihydropyridine-3-carbonitrile

### Intermediate C

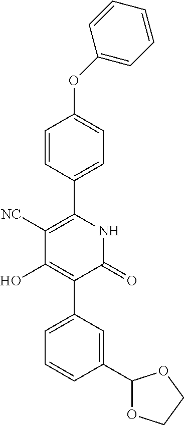 [SMILES: N#Cc1c(-c2ccc(Oc3ccccc3)cc2)[nH]c(=O)c(-c2cccc(C3OCCO3)c2)c1O]

Diethyl 2-(3-(1,3-dioxolan-2-yl)phenyl)malonate (8 mmol) [can be prepared according to a procedure of Beare et al. Journal of Organic Chemistry 2002 67 (2) 541] is combined with 3-amino-3-(4-phenoxyphenyl)acrylonitrile (see WO 2011/019780) and microwaved for 20 minutes. After cooling to room temperature, the mixture is poured into saturated ammonium chloride and extracted with EtOAc. The EtOAc is washed with saturated ammonium chloride, water, and brine. The organic phase is dried over anhydrous magnesium sulfate. Filtration and evaporation gives a residue that is purified by flash chromatography.

### Reference 2

### Synthesis of 5-(3-(1,3-dioxolan-2-yl)phenyl)-6-chloro-4-hydroxy-2-(4-phenoxyphenyl)nicotinonitrile

### Intermediate D

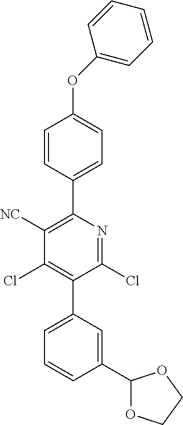 [SMILES: N#Cc1c(-c2ccc(Oc3ccccc3)cc2)nc(Cl)c(-c2cccc(C3OCCO3)c2)c1Cl]

A mixture of 5-(3-(1,3-dioxolan-2-yl)phenyl)-4-hydroxy-6-oxo-2-(4-phenoxyphenyl)-1,6-dihydropyridine-3-carbonitrile C (0.5 mmol) and phosphoryl trichloride (1 mL) is heated to 100° C. for 3 h. The mixture is then concentrated and treated with a slow addition of water to give a suspension. The solid is collected by filtration, washed with water and dried under vacuum to give Intermediate D.

### Reference 3

### Synthesis of 7-(3-(1,3-dioxolan-2-yl)phenyl)-6-chloro-4-(4-phenoxyphenyl)-1H-pyrazolo[4,3-c]pyridin-3-amine

### Intermediate E

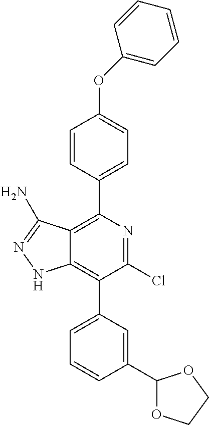 [SMILES: Nc1n[nH]c2c(-c3cccc(C4OCCO4)c3)c(Cl)nc(-c3ccc(Oc4ccccc4)cc3)c12]

A 35% aqueous solution of hydrazine (1.5 mmol) is added to a solution of 5-(3-(1,3-dioxolan-2-yl)phenyl)-6-chloro-4-hydroxy-2-(4-phenoxyphenyl)nicotinonitrile D (0.31 mmol) in ethanol (2 mL). After 18 h at 80° C., the mixture is diluted with EtOAc, washed with water, brine, dried over anhydrous magnesium sulfate, filtered and concentrated. The residue is purified using flash chromatography to give Intermediate E.

### Reference 4

### Synthesis of 7-(3-(1,3-dioxolan-2-yl)phenyl)-4-(4-phenoxyphenyl)-1H-pyrazolo[4,3-c]-pyridin-3-amine

### Intermediate F

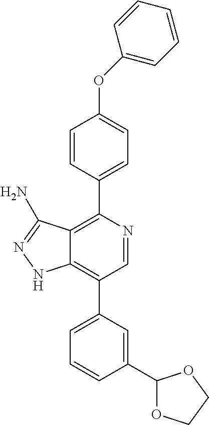 [SMILES: Nc1n[nH]c2c(-c3cccc(C4OCCO4)c3)cnc(-c3ccc(Oc4ccccc4)cc3)c12]

7-(3-(1,3-Dioxolan-2-yl)phenyl)-6-chloro-4-(4-phenoxyphenyl)-1H-pyrazolo-[4,3c]pyridin-3-amine E (5 mmol) and hydrazine (0.1 mol) in THF is heated for 5 days under reflux. After addition of copper (II) sulfate pentahydrate (25 mmol) and water, the mixture is heated under reflux for 45 minutes. The reaction mixture is taken up in EtOAc, washed with water, brine, dried over anhydrous magnesium sulfate, filtered and concentrated. The residue is purified using flash chromatography to give Intermediate F.

### Reference 5

### Synthesis of 3-(3-amino-4-(4-phenoxyphenyl)-1H-pyrazolo[4,3-c]pyridin-7-yl)benzaldehyde

### Intermediate G

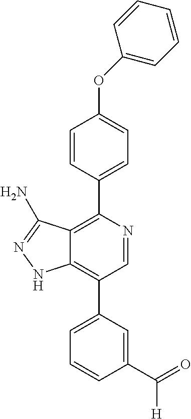 [SMILES: Nc1n[nH]c2c(-c3cccc(C=O)c3)cnc(-c3ccc(Oc4ccccc4)cc3)c12]

To a solution of 7-(3-(1,3-dioxolan-2-yl)phenyl)-4-(4-phenoxyphenyl)-1H-pyrazolo[4,3-c]pyridin-3-amine F (25 mmol) in 50 mL MeOH is added 12 mL of 2N HCl (25 mmol). The mixture is refluxed overnight, then diluted with water and extracted with EtOAc. The organic layers are combined, dried over anhydrous sodium sulfate, filtered, and evaporated in vacuo to give Intermediate G.

### Example 1

### Synthesis of 3-(3-(3-amino-4-(4-phenoxyphenyl)-1H-pyrazolo[4,3-c]pyridin-7-yl)phenyl)-2-cyano-N,N-dimethylacrylamide

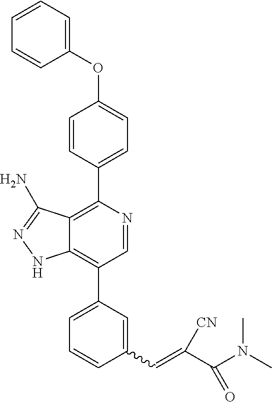 [SMILES: CN(C)C(=O)C(C#N)=Cc1cccc(-c2cnc(-c3ccc(Oc4ccccc4)cc3)c3c(N)n[nH]c23)c1]

To a mixture of 3-(3-amino-4-(4-phenoxyphenyl)-1H-pyrazolo[4,3-c]pyridin-7-yl)benzaldehyde G (1 mmol), in 40 mL ethanol is added 2-cyano-N,N-dimethylacetamide (1.1 mmol), 4 drops of piperidine, and 2 drops HOAc. The resulting mixture is refluxed overnight, and then concentrated and purified by Prep-HPLC providing titled compound.

### Reference 6

### Synthesis of tert-butyl 2-((5-cyano-4-hydroxy-2-oxo-6-(4-phenoxyphenyl)-1,2-dihydropyridin-3-yl)methyl)pyrrolidine-1-carboxylate

### Intermediate I

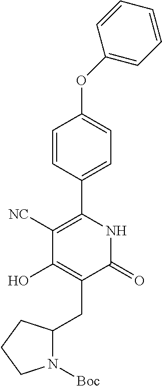 [SMILES: CC(C)(C)OC(=O)N1CCCC1Cc1c(O)c(C#N)c(-c2ccc(Oc3ccccc3)cc2)[nH]c1=O]

Diethyl 2-(1-(tert-butoxycarbonyl)pyrrolidin-2-yl)malonate H is prepared according to a literature procedure (Clemo, G. R. et. al., J. Chem. Soc. 1950 1140). Following conditions described for Intermediate C, H is reacted with 3-amino-3-(4-phenoxyphenyl)acrylonitrile under microwave conditions to give Intermediate I.

### Reference 7

### Synthesis of tert-butyl 2-((2,4-dichloro-5-cyano-6-(4-phenoxyphenyl)pyridin-3-yl)methyl)pyrrolidine-1-carboxylate

### Intermediate J

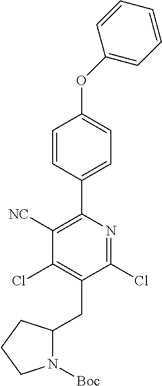 [SMILES: CC(C)(C)OC(=O)N1CCCC1Cc1c(Cl)nc(-c2ccc(Oc3ccccc3)cc2)c(C#N)c1Cl]

Using methodology described for the synthesis of Intermediate D, Intermediate I is treated with phosphoryl chloride under elevated temperature to give J.

### Reference 8

### Synthesis of tert-butyl 2-((3-amino-6-chloro-4-(4-phenoxyphenyl)-1H-pyrazolo[4,3-c]pyridin-7-yl)methyl)pyrrolidine-1-carboxylate

### Intermediate K

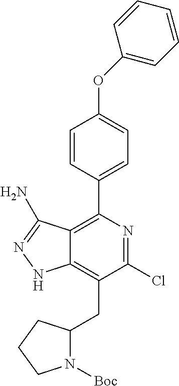 [SMILES: CC(C)(C)OC(=O)N1CCCC1Cc1c(Cl)nc(-c2ccc(Oc3ccccc3)cc2)c2c(N)n[nH]c12]

Following conditions described for Intermediate E, Intermediate J is reacted with hydrazine to give Intermediate K.

### Reference 9

### Synthesis of tert-butyl 2-((3-amino-4-(4-phenoxyphenyl)-1H-pyrazolo[4,3-c]pyridin-7-yl)methyl)pyrrolidine-1-carboxylate

### Intermediate L

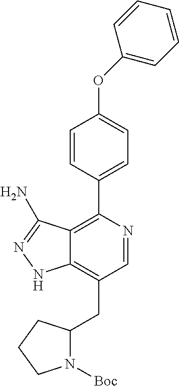 [SMILES: CC(C)(C)OC(=O)N1CCCC1Cc1cnc(-c2ccc(Oc3ccccc3)cc2)c2c(N)n[nH]c12]

Following conditions described for Intermediate F, 3-amino-5-aza-6-chloroindazole is reacted with hydrazine in the presence of copper (II) sulfate to give Intermediate L.

### Reference 10

### Synthesis of 4-(4-phenoxyphenyl)-7-(pyrrolidin-2-ylmethyl)-1H-pyrazolo[4,3-c]pyridin-3-amine

### Intermediate M

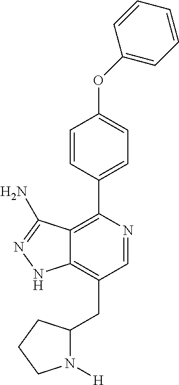 [SMILES: Nc1n[nH]c2c(CC3CCCN3)cnc(-c3ccc(Oc4ccccc4)cc3)c12]

A mixture of tert-butyl 2-((3-amino-4-(4-phenoxyphenyl)-1H-pyrazolo[4,3-c]pyridin-7-yl)methyl)pyrrolidine-1-carboxylate L (1.4 mmol) in dichloromethane (100 mL) and trifluoroacetic acid (20 mL) is stirred at room temperature for 12 h. The reaction mixture is then concentrated under vacuum to give 4-(4-phenoxyphenyl)-7-(pyrrolidin-2-ylmethyl)-1H-pyrazolo[4,3-c]pyridin-3-amine M.

### Example 2

### Synthesis of (2-((3-amino-4-(4-phenoxyphenyl)-1H-pyrazolo[4,3-c]pyridin-7-yl)methyl)pyrrolidine-1-carbonyl)-3-cyclopropylacrylonitrile

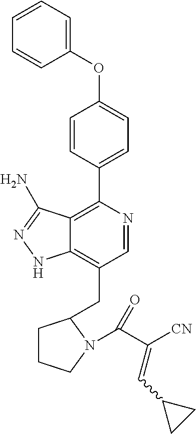 [SMILES: N#CC(=CC1CC1)C(=O)N1CCCC1Cc1cnc(-c2ccc(Oc3ccccc3)cc2)c2c(N)n[nH]c12]

### Step 1

A mixture of 4-(4-phenoxyphenyl)-7-(pyrrolidin-2-ylmethyl)-1H-pyrazolo[4,3-c]pyridin-3-amine M (1.5 mmol), carbonyldiimidazole (2.25 mmol) and 2-cyanoacetic acid (2.24 mmol) in dichloromethane (100 mL) is stirred at room temperature for 24 h. The reaction mixture is diluted with 100 mL of dichloromethane and washed with saturated aqueous ammonium chloride. The organic layer is dried over anhydrous sodium sulfate and concentrated under vacuum. The residue is purified by flash chromatography to give 3-(2-((3-amino-4-(4-phenoxyphenyl)-1H-pyrazolo[4,3-c]pyridin-7-yl)methyl)pyrrolidin-1-yl)-3-oxopropanenitrile which is used immediately in the next step.

### Step 2

A mixture of 3-(2-((3-amino-4-(4-phenoxyphenyl)-1H-pyrazolo[4,3-c]pyridin-7-yl)methyl)pyrrolidin-1-yl)-3-oxopropanenitrile (0.26 mmol), piperidine (0.28 mmol), and cyclopropanecarbaldehyde (0.40 mmol) in methanol (8 mL) is stirred in a sealed tube at room temperature for 24 hr. The resulting mixture is concentrated under vacuum and the residue is purified by flash chromatography to give title compound.

### Reference 11

### Synthesis of 6-chloro-4-(4-phenoxyphenyl)-1H-pyrazolo[4,3-c]pyridin-3-amine

### Intermediate P

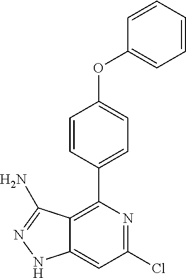 [SMILES: Nc1n[nH]c2cc(Cl)nc(-c3ccc(Oc4ccccc4)cc3)c12]

Following conditions described for Intermediate C, but substituting diethyl 2-(3-(1,3-dioxolan-2-yl)phenyl)malonate with diethylmalonate provides 4-hydroxy-6-oxo-2-(4-phenoxyphenyl)-1,6-dihydropyridine-3-carbonitrile which is converted to 4,6-dichloro-2-(4-phenoxyphenyl)-nicotinonitrile following the procedure described for Intermediate D.

Following conditions described for Intermediate E, 4,6-dichloro-2-(4-phenoxyphenyl)-nicotinonitrile O is reacted with aqueous hydrazine to give 6-chloro-4-(4-phenoxyphenyl)-1H-pyrazolo[4,3-c]pyridin-3-amine P.

### Reference 12

### Synthesis of 3-((3-amino-4-(4-phenoxyphenyl)-1H-pyrazolo[4,3-c]pyridin-6-yl)-amino)benzaldehyde

### Intermediate Q

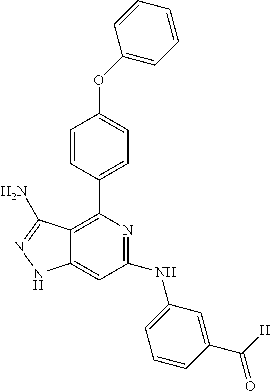 [SMILES: Nc1n[nH]c2cc(Nc3cccc(C=O)c3)nc(-c3ccc(Oc4ccccc4)cc3)c12]

To a solution of 3,1-(1,3-dioxolan-2-yl)benzenamine (50 mmol) and 6-chloro-4-(4-phenoxyphenyl)-1H-pyrazolo[4,3-c]pyridin-3-amine P (50 mmol) in 100 mL methanol is added 25 mL 2N HCl. The resulting mixture is refluxed overnight, then cooled to room temperature, diluted with water and extracted with EtOAc. The organic layers are combined, dried over anhydrous sodium sulfate, filtered and evaporated in vacuo. The residue is purified by flash chromatography to give the title compound Q.

### Example 3

### Synthesis of 3-(3-((3-amino-4-(4-phenoxyphenyl)-1H-pyrazolo[4,3-c]pyridin-6-yl)amino)phenyl)-2-cyano-N,N-dimethylacrylamide

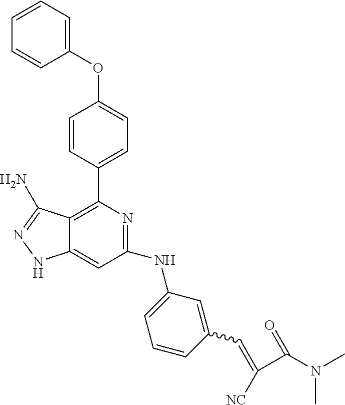 [SMILES: CN(C)C(=O)C(C#N)=Cc1cccc(Nc2cc3[nH]nc(N)c3c(-c3ccc(Oc4ccccc4)cc3)n2)c1]

Using methodology described for the synthesis of Example 1, treatment of 3-((3-amino-4-(4-phenoxyphenyl)-1H-pyrazolo[4,3-c]pyridin-6-yl)amino)benzaldehyde Intermediate Q with cyanoacetamide provides the title compound.

### Reference 13

### Synthesis of 4-hydroxy-2-oxo-6-(4-phenoxyphenyl)-1,2-dihydropyrimidine-5-carbonitrile

### Intermediate R

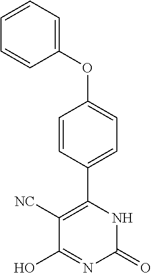 [SMILES: N#Cc1c(O)nc(=O)[nH]c1-c1ccc(Oc2ccccc2)cc1]

Intermediate R is prepared following a procedure detailed in WO 2011/019780.

### Reference 14

### Synthesis of 2-chloro-4-hydroxy-6-(4-phenoxyphenyl)pyrimidine-5-carbonitrile

### Intermediate S

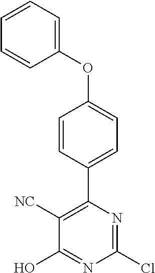 [SMILES: N#Cc1c(O)nc(Cl)nc1-c1ccc(Oc2ccccc2)cc1]

Using methodology described for the synthesis of Intermediate D, 4-hydroxy-2-oxo-6-(4-phenoxyphenyl)-1,2-dihydropyrimidine-5-carbonitrile R is treated with phosphoryl chloride under elevated temperature to give the title compound S.

### Reference 15

### Synthesis of 6-chloro-4-(4-phenoxyphenyl)-1H-pyrazolo[3,4-d]pyrimidin-3-amine

### Intermediate T

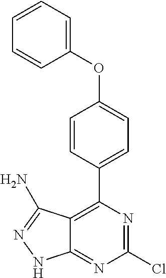 [SMILES: Nc1n[nH]c2nc(Cl)nc(-c3ccc(Oc4ccccc4)cc3)c12]

Following conditions described for Intermediate E, 2-chloro-4-hydroxy-6-(4-phenoxyphenyl)pyrimidine-5-carbonitrile S is reacted with hydrazine to give the title compound T.

### Reference 16

### Synthesis of (3-((3-amino-4-(4-phenoxyphenyl)-1H-pyrazolo[3,4-d]pyrimidin-6-yl)amino)phenyl)methanol

### Intermediate U

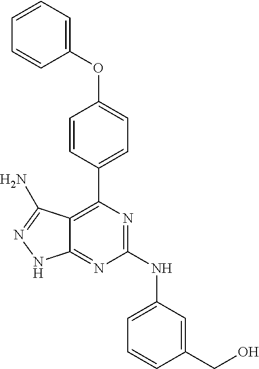 [SMILES: Nc1n[nH]c2nc(Nc3cccc(CO)c3)nc(-c3ccc(Oc4ccccc4)cc3)c12]

To a solution of 6-chloro-4-(4-phenoxyphenyl)-1H-pyrazolo[3,4-d]pyrimidin-3-amine T (3 mmol) in tert-butanol (10 mL) is added (3-aminophenyl)methanol (3.6 mmol), and 1 drop concentrated H₂SO₄. The mixture is refluxed overnight, then diluted with 20 mL water and extracted with EtOAc. The organic phase is dried over anhydrous sodium sulfate, filtered, and concentrated in vacuo. Chromatography gives the title compound U.

### Reference 17

### Synthesis of 3-((3-amino-4-(4-phenoxyphenyl)-1H-pyrazolo[3,4-d]pyrimidin-6-yl)amino)benzaldehyde

### Intermediate V

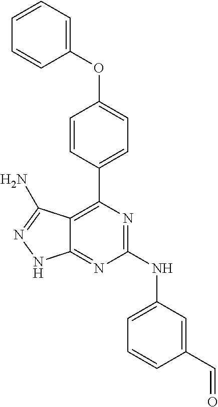 [SMILES: Nc1n[nH]c2nc(Nc3cccc(C=O)c3)nc(-c3ccc(Oc4ccccc4)cc3)c12]

To a solution of (3-((3-amino-4-(4-phenoxyphenyl)-1H-pyrazolo[3,4-d]pyrimidin-6-yl)amino)phenyl)methanol U (2.1 mmol) in absolute dioxane (100 mL) is added MnO₂ (21 mmol). The mixture is refluxed overnight. The solids are filtered off and the filtrate concentrated. The residue is purified by chromatography providing the title compound V.

### Example 4

### Synthesis of 3-(3-((3-amino-4-(4-phenoxyphenyl)-1H-pyrazolo[3,4-d]pyrimidin-6-yl)amino)phenyl)-2-cyano-N,N-dimethylacrylamide

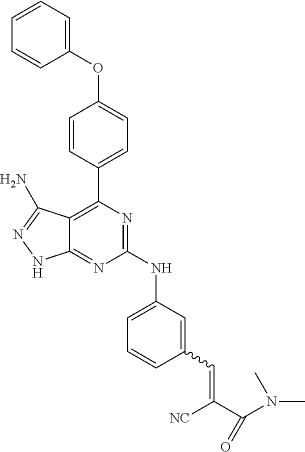 [SMILES: CN(C)C(=O)C(C#N)=Cc1cccc(Nc2nc(-c3ccc(Oc4ccccc4)cc3)c3c(N)n[nH]c3n2)c1]

Using methodology described for the synthesis of Example 1, treatment of the aldehyde Intermediate V with 2-cyano-N,N-dimethylacetamide produces Example 4.

### Reference 18

### Synthesis of 3-chloro-5-(4-phenoxyphenyl)isonicotinonitrile

### Intermediate W

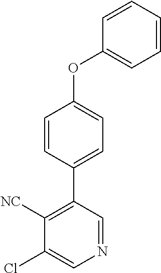 [SMILES: N#Cc1c(Cl)cncc1-c1ccc(Oc2ccccc2)cc1]

Following a procedure outlined in WO 2011/019780, 3,5-dichloroisonicotinonitrile (11 mmol), 3-phenoxyphenylboronic acid (12 mmol), potassium phosphate (21 mmol) and Pd(Ph₃P)₄, is put under a nitrogen atmosphere, and 30 mL DMF is added. The mixture is heated to 110° C. overnight, then cooled to room temperature, diluted with EtOAc, washed with water, and brine. The organics were combined and dried over anhydrous sodium sulfate. The product is purified by chromatography.

### Reference 19

### Synthesis of 2,3-dichloro-5-(4-phenoxyphenyl)isonicotinonitrile

### Intermediate X

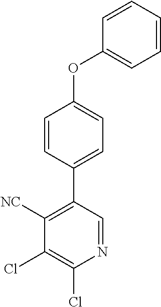 [SMILES: N#Cc1c(-c2ccc(Oc3ccccc3)cc2)cnc(Cl)c1Cl]

Intermediate X is obtained using a two-step procedure detailed in WO 2011/019780, by N-oxidation of 3-chloro-5-(4-phenoxyphenyl)isonicotinonitrile W with m-CPBA and chlorination with POCl₃ of the activated pyridine N-oxide to give a mixture of dichloropyridine isomers. 2,3-Dichloro-5-(3-methoxyphenyl)isonicotinotrile is isolated by recrystallization with a 1:1 ethyl acetate/hexanes mixture.

### Reference 20

### Synthesis of ethyl 3-chloro-4-cyano-5-(4-phenoxyphenyl)-[2,4′-bipyridine]-2′-carboxylate

### Intermediate Y

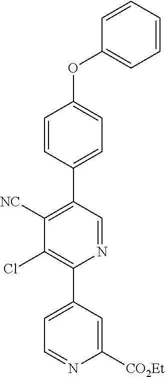 [SMILES: CCOC(=O)c1cc(-c2ncc(-c3ccc(Oc4ccccc4)cc3)c(C#N)c2Cl)ccn1]

A mixture of the 2,3-dichloro-5-(3-methoxyphenyl)isonicotinonitrile (13 mmol), 4-phenoxyphenyl)boronic acid (17 mmol), sodium carbonate (26 mmol), 1,4-dioxane (300 mL), water (75 mL) and Pd(PPh₃)₄ (0.4 mmol) under an inert atmosphere of nitrogen is stirred for 60 h at 90° C. The reaction mixture is then cooled to room temperature and the mixture concentrated under vacuum. The residue is then washed with water and the resulting solids collected by filtration to provide Intermediate Y.

### Reference 21

### Synthesis of ethyl 4-(3-amino-4-(4-phenoxyphenyl)-1H-pyrazolo[3,4-c]pyridin-7-yl)picolinate

### Intermediate Z

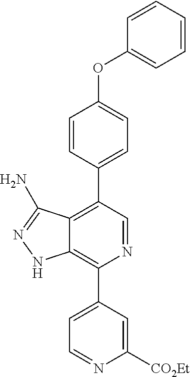 [SMILES: CCOC(=O)c1cc(-c2ncc(-c3ccc(Oc4ccccc4)cc3)c3c(N)n[nH]c23)ccn1]

Following conditions described for Intermediate E, ethyl 3-chloro-4-cyano-5-(4-phenoxyphenyl)-[2,4′-bipyridine]-2′-carboxylate Y is reacted with aqueous hydrazine to give Intermediate Z.

### Reference 22

### Synthesis of (4-(3-amino-4-(4-phenoxyphenyl)-1H-pyrazolo[3,4-c]pyridin-7-yl)pyridin-2-yl)methanol

### Intermediate AA

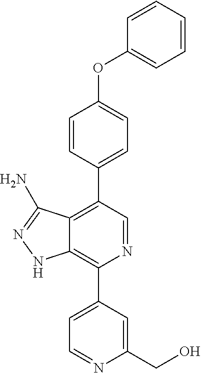 [SMILES: Nc1n[nH]c2c(-c3ccnc(CO)c3)ncc(-c3ccc(Oc4ccccc4)cc3)c12]

Intermediate Z (4.9 mmol) is dissolved in dry THF (125 mL) and treated at 0° C. with diisobutylaluminum hydride (DIBAH) solution (1.0 M in THF; 10 minute addition time) and is stirred for 2 h at room temperature. Additional DIBAH is added at 0° C. and stirring continues at room temperature for 1.5 h. Aqueous work-up and extraction with EtOAc, is followed by drying over anhydrous sodium sulfate, filtration and concentration under reduced pressure which provides the Intermediate AA which is used in the next step without further purification.

### Reference 23

### Synthesis of 2-(4-(3-amino-4-(4-phenoxyphenyl)-1H-pyrazolo[3,4-c]pyridin-7-yl)pyridin-2-yl)acetonitrile

### Intermediate BB

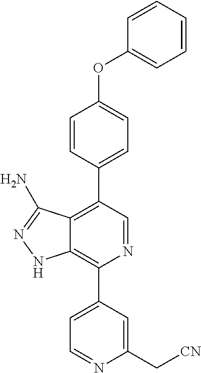 [SMILES: N#CCc1cc(-c2ncc(-c3ccc(Oc4ccccc4)cc3)c3c(N)n[nH]c23)ccn1]

### Step 1

To (4-(3-amino-4-(4-phenoxyphenyl)-1H-pyrazolo[3,4-c]pyridin-7-yl)pyridin-2-yl)methanol AA (10 mmol), DMAP (0.5 mmol) and Et₃N (21 mmol) in dichloromethane (25 mL) is added TosCl (11.5 mmol) in portions. The mixture is stirred at room temperature overnight. The volatile phase is removed under reduced pressure and the residue purified by chromatography to provide 7-(2-(chloromethyl)pyridin-4-yl)-4-(4-phenoxyphenyl)-1H-pyrazolo[3,4-c]pyridin-3-amine.

### Step 2

7-(2-(Chloromethyl)pyridin-4-yl)-4-(4-phenoxyphenyl)-1H-pyrazolo[3,4-c]pyridin-3-amine (4 mmol) and KCN (4.6 mmol) in DMSO (20 mL) is stirred at room temperature for 3 h, quenched with water (50 mL), and extracted with EtOAc. The organic phases are combined, washed with brine, dried over sodium sulfate, filtered and concentrated in vacuo. The residue is purified by chromatography providing the desired compound BB.

### Example 5

### Synthesis of 2-(4-(3-amino-4-(4-phenoxyphenyl)-1H-pyrazolo[3,4-c]pyridin-7-yl)pyridin-2-yl)-3-cyclopropylacrylonitrile

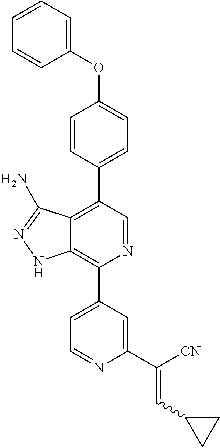 [SMILES: N#CC(=CC1CC1)c1cc(-c2ncc(-c3ccc(Oc4ccccc4)cc3)c3c(N)n[nH]c23)ccn1]

A solution of 2-(4-(3-amino-4-(4-phenoxyphenyl)-1H-pyrazolo[3,4-c]pyridin-7-yl)pyridin-2-yl)acetonitrile BB (1.4 mmol), cyclopropanecarbaldehdye (2.8 mmol), piperidine (1.4 mmol) and HOAc (0.1 mL) in ethanol (20 mL) is refluxed for 2 h. The volatile phase is removed under reduced pressure and the residue submitted to flash chromatography to provide title compound.

### Reference 24

### Synthesis of tert-butyl 3-(3-(4-(3-amino-1H-indazol-4-yl)phenyl)ureido)piperidine-1-carboxylate

### Intermediate DD

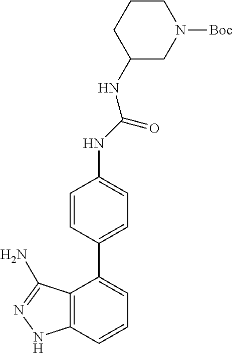 [SMILES: CC(C)(C)OC(=O)N1CCCC(NC(=O)Nc2ccc(-c3cccc4[nH]nc(N)c34)cc2)C1]

4-(4-Aminophenyl)-1H-indazol-3-amine (0.15 mmol) [prepared according to a published method in WO 2004/113304] is taken up in dichloromethane and cooled to 0° C. This mixture is treated with Boc-protected 3-isocyanatopiperidine and stirred at room temperature overnight. The mixture is concentrated in vacuo and purified by prep HPLC to provide title compound DD.

### Reference 25

### Synthesis of 1-(4-(3-amino- H-indazol-4-yl)phenyl)-3-(piperidin-3-yl)urea

### Intermediate EE

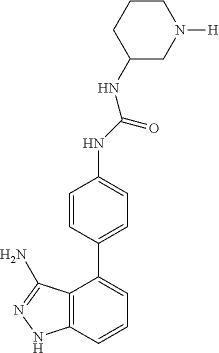 [SMILES: Nc1n[nH]c2cccc(-c3ccc(NC(=O)NC4CCCNC4)cc3)c12]

Following the procedure described for the preparation of Intermediate M, the Boc group on compound DD is removed using trifluoroacetic acid to give the title compound.

### Example 6

### Synthesis of 1-(4-(3-amino-1H-indazol-4-yl)phenyl)-3-(1-(2-cyano-4,4-dimethylpent-2-enoyl)piperidin-3-yl)urea

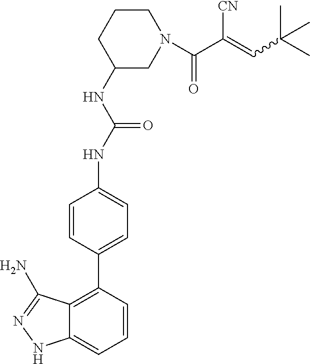 [SMILES: CC(C)(C)C=C(C#N)C(=O)N1CCCC(NC(=O)Nc2ccc(-c3cccc4[nH]nc(N)c34)cc2)C1]

### Step 1

A suspension of pivalaldehyde (0.05 mol), 2-cyanoacetic acid (0.05 mol) and sodium hydroxide (0.1 mol) in methanol (100 mL) is stirred for 2 hours at 40° C. The resulting solution is diluted with 200 mL of EtOAc, the pH adjusted to 4-5 with 2 M HCl, the organic layer is washed with water, and the organic phase combined and dried over sodium sulfate. After filtration, the mixture is concentrated to give 2-cyano-4,4-dimethylpent-2-enoic acid as white solid.

### Step 2

Oxalyl dichloride (0.01 mol) is added dropwise to a solution of 2-cyano-4,4-dimethylpent-2-enoic acid (0.01 mol), with 3 drops of DMF in 40 mL of dichloromethane. The resulting mixture is stirred for 1.5 hours. The solution is concentrated under vacuum to give 2-cyano-4,4-dimethylpent-2-enoyl chloride as a brown oil, which is used directly in the next step.

### Step 3

To the oil residue of 2-cyano-4,4-dimethylpent-2-enoic acid (10 mmol) is added 1-(4-(3-amino-1H-indazol-4-yl)phenyl)-3-(piperidin-3-yl)urea EE (2 mmol) and pyridine (10 mL). The resulting mixture is stirred for 5 hours at 40° C. The reaction mixture is diluted with EtOAc (200 mL) and the organic layer washed with water. The organic phase is dried over sodium sulfate, filtered and concentrated under vacuum to give the title compound.

### Reference 26

### Synthesis of 1-(4-(3-amino-1H-indazol-4-yl)phenyl)-3-(2-methylpyridin-4-yl)urea

### Intermediate FF

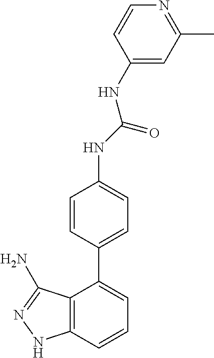 [SMILES: Cc1cc(NC(=O)Nc2ccc(-c3cccc4[nH]nc(N)c34)cc2)ccn1]

Using the procedure described for Intermediate DD, and using 4-isocyanato-2-methylpyridine in place of 3-isocyanatopiperidine, the title compound FF is prepared.

### Reference 27

### Synthesis of (4-(3-(4-(3-amino-1H-indazol-4-yl)phenyl)ureido)pyridin-2-yl)methyl acetate

### Intermediate GG

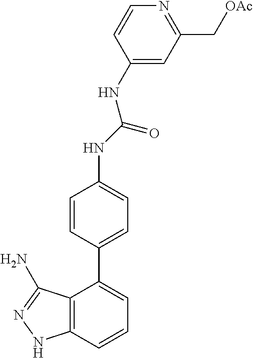 [SMILES: CC(=O)OCc1cc(NC(=O)Nc2ccc(-c3cccc4[nH]nc(N)c34)cc2)ccn1]

### Step 1

To 1-(4-(3-amino-1H-indazol-4-yl)phenyl)-3-(2-methylpyridin-4-yl)urea FF (11 mmol) in 25 mL dichloromethane, add mCPBA (13 mmol) in portions. The reaction mixture is stirred at room temperature overnight, then washed with saturate sodium carbonate and brine sequentially. The organic phase is collected and dried over anhydrous sodium sulfate, then filtered and concentrated in vacuo. This 1-(4-(3-amino-1H-indazol-4-yl)phenyl)-3-(2-methylpyridin-4-yl)urea N-oxide which is used directly in the next step without further purification.

### Step 2

A solution of 1-(4-(3-amino-1H-indazol-4-yl)phenyl)-3-(2-methylpyridin-4-yl)urea N-oxide (9 mmol) in acetic anhydride (10 mL) is refluxed for 2 h. The solution is then poured into ice water and the resulting solution extracted with EtOAc. The organic phase is washed with brine, dried over anhydrous sodium sulfate, filtered and concentrated. The residue is submitted to flash chromatography providing title compound GG.

### Reference 28

### Synthesis of 1-(4-(3-amino-1H-indazol-4-yl)phenyl)-3-(2-(cyanomethyl)pyridin-4-yl)urea

### Intermediate HH

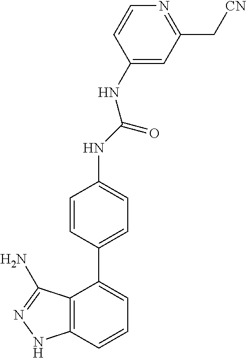 [SMILES: N#CCc1cc(NC(=O)Nc2ccc(-c3cccc4[nH]nc(N)c34)cc2)ccn1]

(4-(3-(4-(3-Amino-1H-indazol-4-yl)phenyl)ureido)pyridin-2-yl)methyl acetate (4-(3-(4-(3-amino-1H-indazol-4-yl)phenyl)ureido)pyridin-2-yl)methyl acetate GG (3 mmol) and KCN (3.3 mmol) in DMSO (10 mL) are stirred at room temperature for 3 h, quenched with water (50 mL), and extracted with EtOAc. The organic phases are combined, washed with brine, dried over sodium sulfate, filtered and concentrated in vacuo. The residue is purified by chromatography providing the title compound HH.

### Example 7

### Synthesis of 1-(4-(3-amino-1H-indazol-4-yl)phenyl)-3-(2-(1-cyano-2-cyclopropylvinyl)-pyridin-4-yl)urea

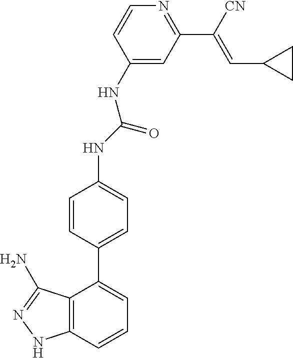 [SMILES: N#C/C(=C\C1CC1)c1cc(NC(=O)Nc2ccc(-c3cccc4[nH]nc(N)c34)cc2)ccn1]

A solution of 1-(4-(3-amino-1H-indazol-4-yl)phenyl)-3-(2-(cyanomethyl)pyridin-4-yl)urea HH (2.1 mmol), cyclopropanecarbaldehdye (3.5 mmol), piperidine (2.1 mmol) and HOAc (0.1 mL) in ethanol (20 mL) is refluxed for 2 h. The volatile phase is removed under reduced pressure and the residue submitted to flash chromatography to provide the title compound.

### Example 8

### Synthesis of 2-(4-(3-amino-4-(4-phenoxyphenyl)-1H-pyrazolo[4,3-c]pyridin-6-yl)piperidine-1-carbonyl)-3-cyclopropylacrylonitrile

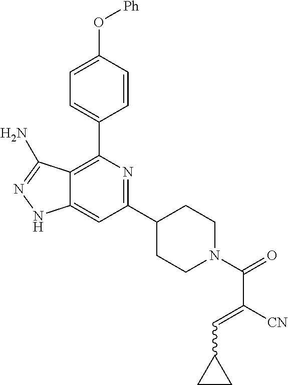 [SMILES: N#CC(=CC1CC1)C(=O)N1CCC(c2cc3[nH]nc(N)c3c(-c3ccc(Oc4ccccc4)cc3)n2)CC1]

### Step 1

To a 100 mL single necked round bottom flask, piperidine-4-carboxylic acid (5.0 g, 38.7 mmol) was taken in water (20 mL). NaOH (3.4 g) was slowly added at RT. The reaction mixture was cooled to 0° C. followed by slow addition of benzylchloroformate (50% in toluene) (14.2 mL, 42.6 mmol)) at the same temperature. After completion of the addition, the reaction mixture was warmed up to RT and stirred for 4 h. The completion of the reaction was monitored on TLC using MeOH:MDC (1:9) as a mobile phase. After completion of the reaction, the reaction mixture was acidify using 6N HCl and the product was extracted in ethyl acetate. The combined organics were washed with water, dried over sodium sulphate and evaporated to afford yellowish oil (crude). The crude compound was purified using column purification by eluting the compound with 3-5% MeOH in MDC to give 1-((benzyloxy)carbonyl)piperidine-4-carboxylic acid 7.8 g

### Step 2

To a 250 mL single necked round bottom flask, 1-((benzyloxy)carbonyl)piperidine-4-carboxylic acid (5.0 g, 19 mmol) was taken in methylene dichloride (50 mL) and DMF (0.1 mL). The reaction mixture was cooled to 0 to 10° C. and oxalyl chloride (2.12 mL, 24.7 mmol) was slowly added. After addition was complete, the reaction mixture was warmed up to RT and stirred for 1 h. The completion of the reaction was monitored by methyl ester formation with MeOH on TLC using MeOH:MDC (1:9) as a mobile phase.

After completion of the reaction, the reaction mixture was evaporated in vacuum under N₂ and toluene was added and distilled to remove traces of oxalyl chloride. The RBF was removed from vacuum under N₂ and the benzyl 4-(chlorocarbonyl)piperidine-1-carboxylate obtained was dissolved in THF and stored.

### Step 3

To a 250 mL three necked round bottom flask, n-BuLi (23% solution in hexane) (18.52 mL) was added to a solution of diisopropyl amine (8.13 mL) in THF (50 mL) at −78° C. The reaction mixture was warmed to 0° C. and stirred for 30 min and again cooled to −78° C. Dry ethyl acetate (6.5 mL) was added and after 30 min at −78° C., a solution of benzyl 4-(chlorocarbonyl)piperidine-1-carboxylate in THF (10 mL) was added and stirred for 1 h at −78° C. The completion of the reaction was monitored on TLC using ethyl acetate:hexanes (3:7) as a mobile phase. After completion of the reaction, the reaction mixtures was warmed to RT and acidify using 1N HCl. The product was extracted in ethyl acetate. The combined organics were washed with water, dried over sodium sulphate and evaporated to afford yellowish oil (crude) product which was purified using column purification by eluting with 10-15% ethyl acetate in hexane to give benzyl 4-(3-ethoxy-3-oxopropanoyl)piperidine-1-carboxylate 7.0 g.

### Step 4

To a 35 mL microwave vessel, 3-amino-3-(4-phenoxyphenyl)acrylonitrile (1.0 g, 4.2 mmol) and benzyl 4-(3-ethoxy-3-oxopropanoyl)piperidine-1-carboxylate (3.5 g, 10.5 mmol) were taken in N,N-dimethylacetamide (7.5 mL) and heated at 250° C. for 10 min in microwave. The completion of the reaction was monitored on TLC using ethyl acetate:hexanes (3:7) as a mobile phase. The above reaction was repeated and combined. The combined reaction mixture was cooled to the room temperature, diluted with water and the solid was filtered and washed with water to get benzyl 4-(5-cyano-4-oxo-6-(4-phenoxyphenyl)-1,4-dihydropyridin-2-yl)piperidine-1-carboxylate 1.8 g.

### Step 5

To a 35 mL sealed tube, benzyl 4-(5-cyano-4-oxo-6-(4-phenoxyphenyl)-1,4-dihydropyridin-2-yl)piperidine-1-carboxylate (1.6 g, 3.17 mmol) and phosphorus oxychloride (0.9 mL, 9.5 mmol) were taken in dry N,N-dimethylformamide (9.5 mL). The solution degassed and heated at 90° C. for 50 min. The completion of the reaction was monitored on TLC using ethyl acetate:hexanes (3:7) as a mobile phase. After completion of the reaction, the reaction mixture was cooled to the room temperature and quenched with saturated sodium bicarbonate solution, followed by extraction with ethyl acetate. The combined organics were dried over sodium sulphate, concentrated to give the crude product which was purified using column purification by eluting with 8-10% ethyl acetate in hexanes to give benzyl 4-(4-chloro-5-cyano-6-(4-phenoxyphenyl)pyridin-2-yl)piperidine-1-carboxylate 1.4 g.

### Step 6

To a 10 mL sealed tube, benzyl 4-(4-chloro-5-cyano-6-(4-phenoxyphenyl)pyridin-2-yl)piperidine-1-carboxylate (0.9 g, 1.72 mmol) and hydrazine hydrate (0.34 mL, 6.87) were taken in dry N,N-dimethylformamide (3 mL). The solution was degassed and heated at 110° C. for 90 min. After cooling to room temperature, TFA (0.92 mL) was added and the mixture was stirred at room temperature for 1.5 h. The completion of the reaction was monitored on TLC using MeOH:MDC (1:9) as a mobile phase. After completion of the reaction, the reaction mixture was quenched with saturated sodium bicarbonate solution, followed by extraction with ethyl acetate. The combined organics were dried over sodium sulphate, concentrated to give the crude product which was purified using column purification by eluting the compound with 5-8% MeOH in MDC to give benzyl 4-(3-amino-4-(4-phenoxyphenyl)-1H-pyrazolo[4,3-c]pyridin-6-yl)piperidine-1-carboxylate 0.60 g.

### Step 7

To a 25 mL round bottom flask, benzyl 4-(3-amino-4-(4-phenoxyphenyl)-1H-pyrazolo[4,3-c]pyridin-6-yl)piperidine-1-carboxylate (0.15 g) was taken in MeOH (1.5 mL) and 6N HCl (2.5 mL) was slowly added at RT and the reaction mixture was refluxed for 3 h. The completion of the reaction was monitored on TLC using MeOH:MDC (1:9) as a mobile phase. After completion of the reaction, reaction mixture was extracted with EtOAc and the organic layer was discarded and the pH of aqueous layer was adjusted to around 11 using addition of aq. NaOH solution at 0-10° C. followed by extraction with ethyl acetate. The combined organics were dried over sodium sulphate, concentrated to give 4-(4-phenoxyphenyl)-6-(piperidin-4-yl)-1H-pyrazolo[4,3-c]pyridin-3-amine 80 mg, which was used as such in next step.

### Step 8

To a 25 mL round bottom flask, 4-(4-phenoxyphenyl)-6-(piperidin-4-yl)-1H-pyrazolo[4,3-c]pyridin-3-amine (0.044 g, 0.4 mmol) and DIPEA (0.068 mL, 1.4 mmol) were taken in dry MDC (1 mL) and the reaction mixture was cooled to 0° C. To the reaction mixture, TBTU (0.175 g, 0.5 mmol) in DMF (0.5 mL) was added dropwise under N₂ atm. After addition was complete the reaction mixture was warmed up to room temperature and 2-cyanoacetic acid (0.20 g) and DIPEA (0.2 mL) dissolved in MDC (2 mL) were added at RT and the reaction mixture was stirred for 16 h. The completion of the reaction was monitored on TLC using MeOH:MDC (1:9) as a mobile phase. After completion of the reaction, the reaction mixture was diluted with MDC (10 mL) and washed with brine. The organic layer was separated and dried over sodium sulphate, concentrated to give the crude product which was purified using column purification by eluting with 2-3% MeOH in MDC to give 3-(4-(3-amino-4-(4-phenoxyphenyl)-1H-pyrazolo[4,3-c]pyridin-6-yl)piperidin-1-yl)-3-oxopropanenitrile 85 mg.

### Step 9

To a 25 mL round bottom flask, 3-(4-(3-amino-4-(4-phenoxyphenyl)-1H-pyrazolo[4,3-c]pyridin-6-yl)piperidin-1-yl)-3-oxopropanenitrile (0.085 g, 0.1 mmol) was taken in dry MeOH (3 mL). Cyclopropylaldehyde (0.015 g, 0.2 mmol) and piperidine (0.0031 g, 0.04 mmol) were added to above under N₂ atm at RT. The reaction mixture was stirred for 1.5 h at RT. The completion of the reaction was monitored on TLC using MeOH: MDC (1:9) as a mobile phase. After completion of the reaction, the reaction mixture was concentrated under vacuum to get the crude product which was purified using column purification by eluting with 0.6-1% MeOH in MDC to give the title compound 15.0 mg. MS (pos. ion) m/z: 505 (M+1).

### Example 9

### Synthesis of 2-(3-(3-amino-4-(4-phenoxyphenyl)-1H-pyrazolo[4,3-c]pyridin-6-yl)piperidine-1-carbonyl)-3-cyclopropylacrylonitrile

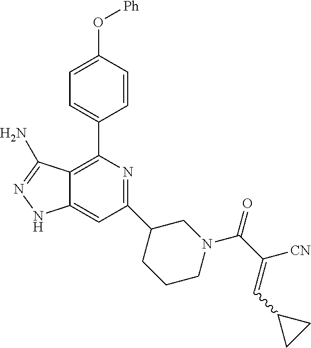 [SMILES: N#CC(=CC1CC1)C(=O)N1CCCC(c2cc3[nH]nc(N)c3c(-c3ccc(Oc4ccccc4)cc3)n2)C1]

### Step 1

Proceeding as described in Example 8, Steps 1-3 above but substituting, piperidine-4-carboxylic acid with piperidine-3-carboxylic acid gave benzyl 3-(3-ethoxy-3-oxopropanoyl)-piperidine-1-carboxylate.

### Step 2

To a 500 mL three necked round bottom flask, n-BuLi (23% solution in hexane) (20.0 mL, 71.8 mmol) was added to a solution of diisopropylamine (7.3 mL, 51.3 mmol) in THF (165 mL) at −78° C. The reaction mixture was warmed to 0° C. and stirred for 20 min and again cooled to −78° C. Acetonitrile (2.7 mL, 51.2 mmol) was added to give a white cloudy solution. After 30 min at −78° C., a solution of 4-phenoxybenzonitrile (10.0 g, 51.2 mmol) in THF (35 mL) was added and stirred for 1.5 h at −78° C. The completion of the reaction was monitored by TLC using ethyl acetate:hexanes (3:7) as a mobile phase. After completion of the reaction, the reaction mixture was warmed to RT and quenched with saturated ammonium chloride solution, water, and 1N HCl (25 mL) followed by extraction with ethyl acetate. The combined organics were washed with 0.1N HCl, followed by brine, dried over sodium sulphate, concentrated to give the crude product which was purified using column purification by eluting the compound with 20-30% ethyl acetate in hexanes to give 3-amino-3-(4-phenoxyphenyl)acrylonitrile.

3-Amino-3-(4-phenoxyphenyl)acrylonitrile was reacted with benzyl 3-(3-ethoxy-3-oxopropanoyl)piperidine-1-carboxylate as described in Example 8, step 4 above to give 3-(5-cyano-4-oxo-6-(4-phenoxyphenyl)-1,4-dihydropyridin-2-yl)piperidine-1-carboxylate which was converted to 2-(3-(3-amino-4-(4-phenoxyphenyl)-1H-pyrazolo[4,3-c]pyridin-6-yl)piperidine-1-carbonyl)-3-cyclopropylacrylonitrile following Steps 5-9, Example 8 above.

MS (pos. ion) m/z: 505 (M+1).

### Example 10

### Synthesis of 2-(2-((3-amino-4-(4-phenoxyphenyl)-1H-pyrazolo[4,3-c]pyridin-6-yl)-methyl)pyrrolidine-1-carbonyl)-3-cyclopropylacrylonitrile

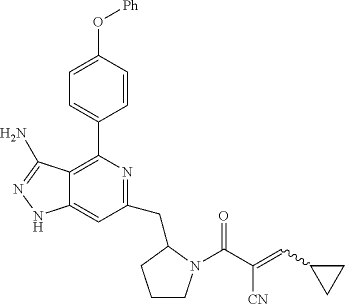 [SMILES: N#CC(=CC1CC1)C(=O)N1CCCC1Cc1cc2[nH]nc(N)c2c(-c2ccc(Oc3ccccc3)cc2)n1]

### Step 1

To a 100 mL single necked round bottom flask, 2-(pyrrolidin-2-yl)acetic acid hydrochloride (2.5 g, 15.09 mmol) was taken in water (16 mL). NaOH (2.65 g) was slowly added at RT. The reaction mixture was cooled to 0° C. followed by slow addition of benzylchloroformate (50% in toluene) (10.3 mL, 30.18 mmol) at the same temperature. After completion of the addition, the reaction mixture was warmed up to RT and stirred for 4 h. The completion of the reaction was monitored on TLC using MeOH:MDC (1:9) as a mobile phase. After completion of the reaction, the reaction mixture was acidify using 6N HCl and the product was extracted in ethyl acetate. The combined organics were washed with water, dried over sodium sulphate and evaporated to afford yellowish oil (crude) product which was purification by column chromatography by eluting the compound with 3-4% MeOH in MDC to give 2-(1-((benzyloxy)carbonyl)pyrrolidin-2-yl)acetic acid 3.5 g.

### Step 2

To a 250 mL single necked round bottom flask, 2-(1-((benzyloxy)carbonyl)pyrrolidin-2-yl)acetic acid (2.5 g) was taken in MDC (10 mL) and DMF (0.5 mL). The reaction mixture was cooled to 0 to 10° C. and oxalyl chloride (0.89 mL) was slowly added. After addition, the reaction mixture was warmed up to RT and stirred for 1 h. The completion of the reaction was monitored by methyl ester formation with MeOH on TLC using MeOH:MDC (1:9) as a mobile phase. After completion of the reaction, the reaction mixture was evaporated in vacuum under N₂ and toluene was added and distilled to remove traces of oxalyl chloride. The RBF was removed from vacuum under N₂ and crude benzyl 2-(2-chloro-2-oxoethyl)pyrrolidine-1-carboxylate obtained was dissolved in THF and stored.

### Step 3

To a 250 mL three necked round bottom flask, n-BuLi (23% solution in hexane) (7.79 mL) was added to a solution of diisopropyl amine (3.38 mL) in THF (20 mL) at −78° C. The reaction mixture was warmed to 0° C. and stirred for 30 min and again cooled to −78° C. Dry ethyl acetate (5.2 mL) was added and after 30 min at −78° C., a solution of benzyl 2-(2-chloro-2-oxoethyl)pyrrolidine-1-carboxylate in THF (5 mL) was added and stirred for 1 h at −78° C. The completion of the reaction was monitored on TLC using Ethyl acetate:Hexanes (3:7) as a mobile phase. After completion of the reaction, the reaction mixtures was warmed to RT and acidify using 1N HCl. The product was extracted in ethyl acetate. The combined organics were washed with water, dried over sodium sulphate and evaporated to afford yellowish oil (crude) product which was purified using column purification by eluting the compound with 12-15% ethyl acetate in hexanes to give benzyl 2-(4-ethoxy-2,4-dioxobutyl)-pyrrolidine-1-carboxylate 2.0 g.

### Step 4

To a 10 mL microwave vessel, 3-amino-3-(4-phenoxyphenyl)acrylonitrile (0.25 g, 1.05 mmol) and benzyl 2-(4-ethoxy-2,4-dioxobutyl)pyrrolidine-1-carboxylate (0.6 g, 1.79 mmol) were taken in N,N-dimethylacetamide (2 mL) and heated at 250° C. for 10 min in microwave. The completion of the reaction was monitored on TLC using MeOH:MDC (0.5:9.5) as a mobile phase. After completion of the reaction, the reaction mixture was cooled to the room temperature, diluted with water and extracted with ethyl acetate. The combined organics were washed with water, dried over sodium sulphate and evaporated to afford crude product. The above reaction was repeated six times. The crude product was combined and purified using column purification by eluting the compound with 0.5-1% MeOH in MDC to give benzyl 2-((5-cyano-4-oxo-6-(4-phenoxyphenyl)-1,4-dihydropyridin-2-yl)methyl)pyrrolidine-1-carboxylate 0.9 g which was converted to the title compound as described in Example 8, Steps 5-9 above. MS (pos. ion) m/z: 505 (M+1).

Using the above method, 2-(3-((3-amino-4-(4-phenoxyphenyl)-1H-pyrazolo[4,3-c]pyridin-6-yl)methyl)-pyrrolidine-1-carbonyl)-4-methylpent-2-enenitrile MS (pos. ion) m/z: 505 (M+1); (Example 10A).

### Example 11

### Synthesis of (R)-2-(3-(3-amino-4-(4-phenoxyphenyl)-1H-pyrazolo[4,3-c]pyridin-6-yl)piperidine-1-carbonyl)-4-methylpent-2-enenitrile

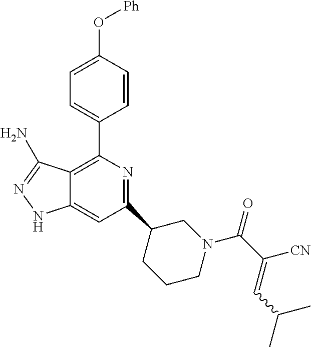 [SMILES: CC(C)C=C(C#N)C(=O)N1CCC[C@@H](c2cc3[nH]nc(N)c3c(-c3ccc(Oc4ccccc4)cc3)n2)C1]

### Step 1

Proceeding as described in Example 8, Steps 1-3 above but substituting piperidine-4-carboxylic acid with (R)-piperidine-3-carboxylic acid gave (R)-benzyl 3-(3-ethoxy-3-oxopropanoyl)piperidine-1-carboxylate.

### Step 2

As described below, the method of Example 8, Step 4 was modified slightly by running the reaction at 150° C. to afford enantio enriched compounds.

To a 35 mL microwave vessel, 3-amino-3-(4-phenoxyphenyl)acrylonitrile (0.25 g, 0.00105 mole) and (R)-benzyl 3-(3-ethoxy-3-oxopropanoyl)piperidine-1-carboxylate (0.706 g, 0.002118 mole) were taken in dry N,N-dimethylacetamide (2 mL) and heated at 150° C. for 30 minutes and further for 15 minutes in microwave at the same temperature. The completion of the reaction was monitored on TLC using MeOH:DCM (1:9) as a mobile phase. After completion of the reaction, the reaction mixture was cooled to the room temperature, diluted with water (20 mL) and extracted with ethyl acetate. The combined organics were washed with water, dried over sodium sulfate and evaporated to afford black oil (crude) product which was subjected to column purification. The pure compound was eluted using 0.5% MeOH in DCM to yield 0.125 g pure (R)-benzyl 3-(5-cyano-4-oxo-6-phenyl-1,4-dihydropyridin-2-yl)piperidine-1-carboxylate with 23.43% yield.

### Steps 3

(R)-Benzyl 3-(5-cyano-4-oxo-6-phenyl-1,4-dihydropyridin-2-yl)piperidine-1-carboxylate was converted to (R)-4-(4-phenoxyphenyl)-6-(piperidin-3-yl)-1H-pyrazolo[4,3-c]pyridin-3-amine following Steps 5-7, Example 8 above.

### Step 4

To a 25 mL round bottom flask, (R)-4-(4-phenoxyphenyl)-6-(piperidin-3-yl)-1H-pyrazolo[4,3-c]pyridin-3-amine (0.1 g, 0.00025 mole) and 2-cyano-4-methylpent-2-enoic acid (0.046 g, 0.00033 mole) were taken in dry DCM (1 mL) and the solution was cooled to 0° C. under argon atmosphere. To above, PyBrOP (0.133 g, 0.000285 mole) was added slowly followed by triethylamine (0.1 mL, 0.000778 mole) at 0° C. The completion of the reaction was monitored on TLC using MeOH:DCM (1:9) as a mobile phase. After completion of the reaction the reaction, the crude material was directly loaded onto a column packed with 100-200 mesh sized silica eluting with neat ethyl acetate to afford 38 mg of the title compound. MS (pos. ion) m/z: 507 (M+1).

Using the above method, following compounds were prepared:
(S)-2-(3-(3-amino-4-(4-phenoxyphenyl)-1H-pyrazolo[4,3-c]pyridin-6-yl)piperidine-1-carbonyl)-4-methylpent-2-enenitrile, MS (pos. ion) m/z: 507 (M+1) (Example 11A);
(S)-2-(2-(3-amino-4-(4-phenoxyphenyl)-1H-pyrazolo[4,3-c]pyridin-6-yl)pyrrolidine-1-carbonyl)-4-methylpent-2-enenitrile, MS (pos. ion) m/z: 507 (M+1) (11B); and
(R)-2-(2-(3-amino-4-(4-phenoxyphenyl)-1H-pyrazolo[4,3-c]pyridin-6-yl)pyrrolidine-1-carbonyl)-4-methylpent-2-enenitrile, MS (pos. ion) m/z: 507 (M+1) (11C).

### BIOLOGICAL EXAMPLES

### Example 1

### Btk Enzymatic Activity Assay

A Caliper-based kinase assay (Caliper Life Sciences, Hopkinton, Mass.) was used to measure inhibition of Btk kinase activity of a compound of Formula (I). Serial dilutions of test compounds were incubated with human recombinant Btk (2 nM), ATP (40 μM) and a phosphoacceptor peptide substrate FAM-GEEPLYWSFPAKKK-NH₂ (1 μM) at room temperature for 3 h. The reaction was then terminated with EDTA, final concentration 20 mM and the phosphorylated reaction product was quantified on a Caliper Desktop Profiler (Caliper LabChip 3000). Percent inhibition was calculated for each compound dilution and the concentration that produced 50% inhibition was calculated. The IC₅₀ for the compounds of the disclosure is provided in table below.

| Example # | IC₅₀ (um) |
|---|---|
| 8 | 0.0020 |
| 9 | 0.0011 |
| 10 | 0.0020 |
| 10A | 0.0012 |
| 11 | 0.0093 |
| 11A | 0.0155 |
| 11B | 0.0130 |
| 11C | 0.0136 |

### Example 2

### Tyrosine Kinase TR-FRET Assay

Inhibition of tyrosine kinase enzymatic activity by compounds is measured using time-resolved fluorescence resonance energy transfer (TR-FRET) (Invitrogen pamphlet: Optimization of a LanthaScreen Kinase assay for BTK). Here, a signal is observed only when a Europium-coupled phophotyrosine antibody binds the phosphorylated peptide. Compounds are first prepared in 100% DMSO and serially diluted 10 times via 3-fold dilution. 2.5 μl of inhibitor at 4-fold the final assay concentration is next transferred to the 384-well assay plate (Corning Catalog #3676). A solution of 2-fold the final concentration of appropriate kinase enzyme and Alexafluor 647-coupled peptide substrate (Invitrogen Catalog #5693) is next prepared in advance in a kinase buffer of 50 mM Hepes pH 7.5, 10 mM MgCl2, and 1 mM EGTA. For this solution, the final concentration of the appropriate kinase and peptide is typically 1 nM and 100 nM, respectively. 5 μL of this 2-fold mix of kinase and peptide is added as the second step of the procedure to the 384-well assay plate. To initiate the enzymatic reaction, 2.5 μl of a 4-fold excess ATP solution in kinase buffer is added to the 384-well assay plate. Final ATP concentration is typically set to the Km for ATP. The reaction is allowed to proceed for 60 minutes. During the kinase reaction, a stop solution consisting of EDTA and a Europium-containing phosphotyrosine antibody (Invtrogen Catalog #5692) is prepared in TR-FRET dilution buffer (Invitrogen Catalog #3574). The stop solution contains an EDTA concentration of 20 mM and an antibody concentration of 4 nM. After the 60 minute reaction, 10 μl of the stop solution is added to all wells. Each well is mixed and incubated for 30 minutes at room temperature. Plates are read on a Perkin Elmer Envision TR-FRET plate reader under LanthaScreen settings. Excitation wavelength is 337 nm and Emission wavelengths are 620 nm and 665 nm. Data are acquired as the ratio of emission at 665 nm/emission at 620 nm and plotted as a function of compound concentration to ascertain compound potency. Here, a signal is observed only when a Europium-coupled phophotyrosine antibody binds the phosphorylated peptide.

### Example 3

### BTK Radiometric Enzyme Assay

BTK activity is measured by product formation based on the incorporation of ³³PO4 from [33P]ATP into a biotin-tagged substrate peptide (see Dinh M., et. al. Activation mechanism and steady state kinetics of Bruton's tyrosine kinase. J. Biol. Chem. 282:8768-76. 2007). The peptide Is isolated from unreacted [³³P]ATP using streptavidin-coated beads. Each well of a 96-well V bottom plate (Greiner, Monroe, N.C.), contains assay buffer (8 mM imidazole, pH 7.2, 8 mM glycerol 2-phosphate, 200 uM EGTA, 20 mM MgCl2, 1 mM MnCl2, 0.1 mg/mL bovine serum albumin, and 2 mM dithiothreitol) which was combined to 40 ul with a mixture of substrates dissolved in assay buffer such that the final concentrations were 1 uCi of [³³P]ATP, 100 uM ATP, and peptide substrate (biotin-Aca-AAAEEIYGEI-NH2). Initiation of the reaction is by addition of BTK to a final concentration of 10 nM. The reaction is incubated at 30° C. for 15 min. The reaction is stopped by transferring 25 ul of sample to a 96-well 1.2-um hydrophilic polyvinylidene difluoride filter plate (Millipore, Billerica, Mass.) containing 10% streptavidin-Sepharose beads (GE Healthcare) dissolved in phosphate-buffered saline plus 50 mM EDTA. Filter plates are washed with 2 M NaCl, then with 2 M NaCl with 1% phosphoric acid, and then with H₂O. Plates were allowed to dry and microscint-20 (PerkinElmer Life Sciences, Boston, Mass.) was added. The [³³P]phosphoproduct is detected by a top-count scintillation counter. The enzyme activity is calculated for each data point. The corrected number of counts in each well is determined by subtracting the background counts from the measured counts. This value is then divided by the total number of counts that were originally present in the solution (determined by spotting and counting an equivalent volume of unwashed sample on a filter plate) and multiplied by the concentration of ATP in solution to give the concentration of phosphorylated product formed. Selectivity for BTK will be determined using commercially available kinase cross-screening services (DiscoveRx, San Diego, Calif.).

### Example 4

### BTK TR-FRET Assay

Inhibition of BTK enzymatic activity by compounds is measured using time-resolved fluorescence resonance energy transfer (TR-FRET) (Invitrogen pamphlet: Optimization of a LanthaScreen Kinase assay for BTK). Here, a signal is observed only when a Europium-coupled phophotyrosine antibody binds the phosphorylated peptide. Compounds are first prepared in 100% DMSO and serially diluted 10 times via 3-fold dilution. 2.5 ul of inhibitor at 4-fold the final assay concentration is next transferred to the 384-well assay plate (Corning Catalog #3676). A solution of 2-fold the final concentration of BTK enzyme (Invitrogen Catalog #PV3363) and Alexafluor 647-coupled peptide substrate (Invitrogen Catalog #5693) is next prepared in advance in a kinase buffer of 50 mM Hepes pH 7.5, 10 mM MgCl2, and 1 mM EGTA. For this solution, the final concentration of BTK and peptide is typically 1 nM and 100 nM, respectively. 5 uL of this 2-fold mix of BTK and peptide is added as the second step of the procedure to the 384-well assay plate. To initiate the enzymatic reaction, 2.5 ul of a 4-fold excess ATP solution in kinase buffer is added to the 384-well assay plate. Final ATP concentration is typically set to the Km for ATP of 100 uM. The reaction is allowed to proceed for 60 minutes. During the kinase reaction, a stop solution consisting of EDTA and a Europium-containing phosphotyrosine antibody (Invtrogen Catalog #5692) is prepared in TR-FRET dilution buffer (Invitrogen Catalog #3574). The stop solution contains an EDTA concentration of 20 mM and an antibody concentration of 4 nM. After the 60 minute reaction, 10 ul of the stop solution is added to all wells. Each well is mixed and incubated for 30 minutes at room temperature. Plates are read on a Perkin Elmer Envision TR-FRET plate reader under LanthaScreen settings. Excitation wavelength is 337 nm and Emission wavelengths are 620 nm and 665 nm. Data are acquired as the ratio of emission at 665 nm/emission at 620 nm and plotted as a function of compound concentration to ascertain compound potency. Here, a signal is observed only when a Europium-coupled phophotyrosine antibody binds the phosphorylated peptide.

### Example 5

### Cellular BTK Activity Measured by Reporter Assay in Ramos Cells

The beta lactamase-based select-screen reporter assay is used to measure BTK cell-based activity (Invitrogen Selectscreen Screening Protocol and Assay Conditions document. Revised 8 Feb. 2010). 32 μL of NFAT-bla RA1 (Invitrogen) cells diluted in Assay Media to appropriate cell density are added to the Poly-D-Lysine assay plate containing 4 μL of a 10× serial dilution of a BTK control compound or test compounds. Pre-incubation at 37° C./5% CO2 in a humidified incubator with compounds and control inhibitor titration is for 30 minutes. 4 μL of 10× control activator Goat anti-Human IgM at the pre-determined EC80 concentration is added to wells containing the control inhibitor or compounds. The plate is incubated for 5 hours at 37° C./5% CO₂ in a humidified incubator. 8 μL of 1 μM Substrate Loading Solution is added to each well and the plate is incubated for 2 hours at room temperature. The plate is read on a fluorescence plate reader and the data is analyzed. A response ratio is calculated from the emissions of cleaved and uncleaved substrate. The response ratio of wells with compound dilutions is compared with wells that contain only DMSO to calculate the percent inhibition at each compound concentration. A dose response curve is constructed and an IC₅₀ is calculated.

### Example 6

### Blockade of CD69 Expression in Whole Blood Samples

Activation of the B cell receptor leads to increased BTK activity, calcium mobilization and B cell activation (see Honigberg L. A., et. al. Proc Natl Acad Sci USA. 107:13075-80. 2010). BTK inhibitors have been shown to block B cell activation as measured by CD69 expression (see Karp, R., et. al. Inhibition of BTK with AVL-292 Translates to Protective Activity in Animal Models of Rheumatoid Arthritis. Inflammation Research Association Meeting, September, 2010). We used expression of CD69 following B cell activation as a measure of BTK activity in whole blood. Aliquots of whole blood are pre-incubated with serial dilutions of test compound for 30 minutes followed by activation with anti-IgM (goat Fab′2, 50 ug/mL). Samples are incubated overnight at 37 C and then stained with PE labeled anti-CD20 and APC labeled anti-CD69 (BD Pharmingen) for 30 minutes according to the manufacturer's directions. Whole blood is then lysed and cells gated on CD20 expression are quantified for CD 69 expression by FACS. The percent inhibition is calculated based on a DMSO control for no inhibition and plotted as a function of test compound concentration from which an IC50 value is calculated.

### Example 7

### Inhibition of Mouse Collagen-Induced Arthritis

Inhibition of murine collagen-induced arthritis (mCIA) is a standard animal disease model for rheumatoid arthritis. Previous studies have demonstrated that inhibition of BTK is efficacious in blocking mCIA (see Honigberg L. A., et. al. Proc Natl Acad Sci USA. 107:13075-80. 2010). Starting on day 0 DBA/1 mice are injected with an emulsion of Type II collagen in Complete Freund's Adjuvant. Mice are boosted 21 days later to synchronize development of disease. After development of mild disease, animals are enrolled in the study and randomized. Dosing is oral, Q.D. typically for 11 days with test compound or dexamethasone (0.2 mg/kg) as control. One group receives vehicle alone. Clinical scoring (0-4) is based on the extent of swelling and severity of arthritis. Scores for all four paws are added for maximum score of 16. Anti-collagen antibodies and total Ig are measured for each animal by Elisa at the end of the study (Bolder BioPath, Boulder, Colo.).

### Example 8

### Recovery of Kinase Activity Upon Dialysis

Standard experimental methods to establish reversibility are known in the art. Protein dialysis is one such method. A solution containing a protein kinase that is inhibited by a compound of Formula I may be subjected to extensive dialysis to establish if the kinase inhibitor is reversible. Partial or complete recovery of protein kinase activity over time during dialysis is indicative of reversibility.

### Method:

A compound of Formula I described herein (1 uM) is added to a solution of protein kinase (50 nM, pre-activated if necessary) in a buffer containing 20 mM Hepes [pH 8.0], 10 mM MgCl2, 2.5 mM tris(2-carboxyethyl)phosphine (TCEP), 0.25 mg/mL BSA, and 100 uM ATP. After 60 min at rt, the reactions is transferred to a dialysis cassette (0.1-0.5 mL Slide-A-Lyzer, MWCO 10 kDa, Pierce) and dialyzed against 2 L of buffer (20 mM Hepes [pH 8.0], 10 mM MgCl2, 1 mM DTT) at 4° C. The dialysis buffer is exchanged after 2 h, and then is exchanged every 24 h until the end of the experiment. Aliquots are removed from the dialysis cassettes every 24 h, flash frozen in liquid nitrogen, and subsequently analyzed for protein kinase activity in triplicate. Kinase activity for each sample is normalized to the DMSO control for that time point and expressed as the mean±SD.

Results: Kinase activity recovers from inhibition by reversible kinase inhibitors upon dialysis. Upon extensive dialysis at 4° C. or at room temperature, kinase activity partially or completely recovers in a time-dependent manner from inhibition by an excess (20 equiv, 1.0 uM) of reversible kinase inhibitor.

### Example 9

### Mass Spectral Analysis

A protein kinase that is inhibited by compound of Formula I may be subjected to mass spectral analysis to assess the formation of permanent, irreversible covalent adducts. Suitable analytical methods to examine intact full protein or peptide fragments generated upon tryptic cleavage of the protein kinase are generally known in the art. Such methods identify permanent, irreversible covalent protein adducts by observing a mass peak that corresponds to the mass of a control sample plus the mass of an irreversible adduct. Two such methods are described below.

### Mass Spectral Analysis of Intact Full Kinase

### Method:

A protein kinase (5 uM) is incubated with a compound of Formula I (25 uM, 5 equiv) for 1 h at room temperature in buffer (20 mM Hepes [pH 8.0], 100 mM NaCl, 10 mM MgCl2). A control sample is also prepared which does not have a compound of Formula I. The reaction is stopped by adding an equal volume of 0.4% formic acid, and the samples are analyzed by liquid chromatography (Microtrap C18 Protein column [Michrom Bioresources], 5% MeCN, 0.2% formic acid, 0.25 mL/min; eluted with 95% MeCN, 0.2% formic acid) and in-line ESI mass spectrometry (LCT Premier, Waters). Molecular masses of the protein kinase and any adducts may be determined with MassLynx deconvolution software.

Results: High-resolution intact mass spectrometry analysis of a kinase that is inhibited by a compound of Formula I will reveal a spectrum similar to the kinase in the absence of inhibitor (e.g. control sample). There will be no formation of a new peak in the mass spectrum corresponding to the molecular mass of the kinase plus the molecular mass of the compound of Formula I. On the basis of this experiment no permanent, irreversible protein adduct will be apparent to one skilled in the art.

### Mass Spectral Analysis of Kinase Tryptic Digest

### Method:

A protein (10-100 pmols) is incubated with a compound of Formula I (100-1000 pmols, 10 equiv) for 3 hrs prior to tryptic digestion. Iodoacetamide may be used as the alkylating agent after compound incubation. A control sample is also prepared which does not the compound of Formula I. For tryptic digests a 1 ul aliquot (3.3 pmols) is diluted with 10 ul of 0.1% TFA prior to micro C18 Zip Tipping directly onto the MALDI target using alpha cyano-4-hydroxy cinnamic acid as the desorption matrix (5 mg/mol in 0.1% TFA:Acetonitrile 50:50) or Sinapinic acid as the desorption matrix (10 mg/mol in 0.1% TFA:Acetonitrile 50:50).

Results: High-resolution mass spectrometry analysis of the tryptic fragments of a kinase that is inhibited by a compound of Formula I will reveal a spectrum similar to the kinase in the absence of inhibitor (e.g. control sample). There will be no evidence of any modified peptides that are not present in the control sample. On the basis of this experiment, no permanent, irreversible protein adducts will be apparent to one skilled in the art. Cellular assays are also optionally used to assess the inhibiting properties of a compound of Formula I provided herein or embodiments thereof. Cellular assays include cells from any appropriate source, including plant and animal cells (such as mammalian cells). The cellular assays are also optionally conducted in human cells. Cellular assays of BTK inhibition are well known in the art, and include methods in which an inhibitor is delivered into the cell (e.g. by electroporation, passive diffusion, microinjection and the like) and an activity endpoint is measured, such as the amount of phosphorylation of a cellular substrate, the amount of expression of a cellular protein, or some other change in the cellular phenotype known to be affected by the catalytic activity of BTK. For example, phosphorylation of a particular cellular substrate is optionally assessed using a detection antibody specific or the phosphorylated cellular substrate followed by western blotting techniques and visualization using any appropriate means (e.g. fluorescent detection of a fluorescently labeled antibody).

Measuring the reduction in the BTK catalytic activity in the presence of an inhibitor disclosed herein relative to the activity in the absence of the inhibitor is optionally performed using a variety of methods known in the art, such as the assays described in the Examples section below. Other methods for assaying BTK activity are known in the art.

### Example 10

### Determination of Drug-Kinase Residence Time . . . Drug Off-Rate Assay

The following is a protocol to distinguish whether a compound displays a slow or non-existent dissociation rate from BTK, such as typically would occur if a covalent bond is formed between the compound and the target. The read-out for slow dissociation is the ability of the compound of interest to block binding of a high affinity fluorescent tracer molecule to the kinase active site, as detected using time-resolved fluorescence resonance energy transfer (TR-FRET). The experiment was conducted in a buffer consisting of 50 mM Hepes pH 7.5, 10 mM MgCl2, 0.01% Triton X-100, and 1 mM EGTA.

The first step of the procedure was incubation of 500 nM BTK (Invitrogen Cat. #PV3587) with 1.5 uM of a compound of Formula (IA) for 30 minutes in a volume of 10 uL. The mixture was then diluted 5-fold by addition of 40 uL of buffer. A 10 uL volume of the diluted kinase/compound solution was then added to a well of a small volume 384 well plate (such as Greiner Cat. #784076). In order to probe for reversibility of the kinase-compound binding interaction, a competition solution containing both a high affinity fluorescent tracer and an antibody coupled to Europium was prepared. For BTK, the competition solution contained 1.5 uM Tracer 178 (Invitrogen Cat. #PV5593), which is a proprietary high affinity ligand for BTK coupled to the fluorophore AlexaFluor 647. The competition solution also contained 80 nM of an Anti-polyhistidine antibody coupled to Europium (Invitrogen Cat. #PV5596) which is designed to bind the polyhistidine purification tag in BTK.

After addition of 10 uL of the competition solution to the Greiner plate, the mixture was incubated for one hour or greater to allow time for dissociation of non-covalent inhibitors and binding of the high affinity tracer. It was expected that covalent and slow dissociating inhibitors will block binding of the tracer while rapidly dissociating non-covalent inhibitors will not. Binding of the tracer to BTK was detected using TR-FRET between the Europium moiety of the Anti-histidine antibody and the AlexaFluor 647 group of Tracer 178. Binding was evaluated using a Perkin Elmer Envision instrument (Model 2101) equipped with filters and mirrors compatible with LANCE-type TR-FRET experiments. Data were plotted at percentage of signal obtained in the absence of competitor compound. The background signal was obtained by omission of BTK from the reaction.

### Example 11

### Reversibility of Binding

The following approach was developed to differentiate compounds that form irreversible bonds with their targets, such as acrylamide compounds, from compound that bind reversibly. Reactions are prepared with the protein target at a higher concentration than the compounds of interest. Both irreversible and reversible compounds bind the target and become depleted from solution. The reactions are then treated with perturbations including both denaturation with 5 M guanidine hydrochloride and digestion with trypsin, disrupting proper folding of the target. It is found that the perturbation returns reversible compounds to solution due to dissociation from the target while irreversible compounds remain bound to the target. The concentration of compound in solution is assessed both preceding and following perturbation using high performance liquid chromatography (HPLC) coupled to tandem mass spectrometry.

### Formulation Examples

The following are representative pharmaceutical formulations containing a compound of Formula (I′) or (I).

### Tablet Formulation

The following ingredients are mixed intimately and pressed into single scored tablets.

| | Quantity per tablet |
|---|---|
| Ingredient | mg |
| compound of this disclosure | 400 |
| cornstarch | 50 |
| croscarmellose sodium | 25 |
| lactose | 120 |
| magnesium stearate | 5 |

### Capsule Formulation

The following ingredients are mixed intimately and loaded into a hard-shell gelatin capsule.

| | Quantity per capsule |
|---|---|
| Ingredient | mg |
| compound of this disclosure | 200 |
| lactose spray dried | 148 |
| magnesium stearate | 2 |

### Injectable Formulation

Compound of the disclosure (e.g., compound 1) in 2% HPMC, 1% Tween 80 in DI water, pH 2.2 with MSA, q.s. to at least 20 mg/mL

The foregoing disclosure has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the disclosure should, therefore, be determined not with reference to the above description, but should instead be determined with reference to the following appended claims, along with the full scope of equivalents to which such claims are entitled.

All patents, patent applications and publications cited in this application are hereby incorporated by reference in their entirety for all purposes to the same extent as if each individual patent, patent application or publication were so individually denoted.

What is claimed:

1. A compound of Formula (I′): 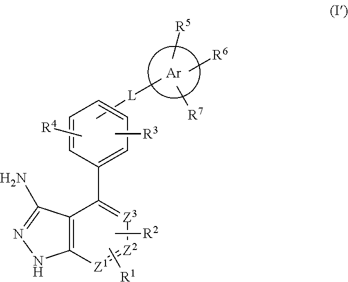
wherein:
Z¹, Z², and Z³ are —N— or CH, provided that not more than two of Z¹, Z², and Z³ are simultaneously N;
L is —O—, —C(O)—, —CH₂—, —S—, —S(O)—, —S(O₂)—, —N(R)—, —N(R)C(O)—, —C(O)N(R)—, —N(R′)S(O₂)—, —S(O₂)N(R′)—, or —N(R)C(O)N(R′)—, where each R and R′ is independently hydrogen, alkyl or cycloalkyl;
Ar is aryl, heteroaryl, cycloalkyl or heterocyclyl;
one of R¹ and R⁵ is hydrogen, alkyl, hydroxy, alkoxy, halo, haloalkyl, or haloalkoxy and the other of R¹ and R⁵ is —Z-(EWG′)-C(R^{b})═CHR^{c} where Z is bond, NR^{a} (where R^{a} is hydrogen or alkyl), —O—, —S—, —S(O)—, —S(O₂)— alkylene, cycloalkylene, heteroalkylene, -(Z^{a})ₙ₁-aryl, or -(Z^{a})ₙ₁-heteroaryl (wherein n1 is 0 or 1, Z^{a} is NR^{a} (where R^{a} is hydrogen or alkyl), —O—, S, SO, SO₂, alkylene, or heteroalkylene and aryl or heteroaryl is optionally substituted with one or two substituents independently selected from hydrogen, halo, alkyl, alkoxy, alkylthio, haloalkyl, or haloalkoxy), EWG′ is a bond, —CH(haloalkyl), —NR′—, —S(O₂)—, —S(O)—, —CO—, —NR′CO—, —NR′SO₂—, 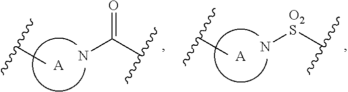
heteroaryl, or aryl; wherein each R′ is independently hydrogen, alkyl, substituted alkyl, or cycloalkyl; ring A is heterocycloamino where the carbonyl and sulfonyl groups are attached to —C(R^{b})═CHR^{c}; and unless defined otherwise, the heterocycloamino, aryl and heteroaryl are optionally substituted with one, two or three substituents independently selected from hydrogen, alkyl, alkoxy, hydroxyl, cyano, nitro, halo, haloalkyl, haloalkoxy, alkylthio, alkylsulfonyl, carboxy, alkoxycarbonyl, aminocarbonyl or aminosulfonyl, R^{b} is cyano, nitro, halo, haloalkyl, haloalkoxy, alkylthio, or alkylsulfonyl and R^{c} is alkyl, substituted alkyl, haloalkoxy, cycloalkyl, cycloalkyleneNR^{d}R^{e} or cycloalkylene(alkylene)NR^{d}R^{e} (where R^{d} and R^{e} are independently hydrogen, alkyl, or cycloalkyl) or 3 to 6 membered saturated monocyclic heterocyclyl containing one or two heteroatoms selected from N, O, or S and optionally substituted with one or two substituents selected from hydroxy, alkyl or fluoro;
R² is hydrogen, alkyl, hydroxy, alkoxy, cyano, halo or haloalkyl;
R³ is hydrogen, alkyl, cycloalkyl, hydroxy, alkoxy, cyano, halo, haloalkyl or haloalkoxy;
R⁴ is hydrogen, alkyl, alkynyl, cycloalkyl, alkylamino, dialkylamino, alkylthio, alkylsulfonyl, carboxy, alkoxycarbonyl, alkylaminosulfonyl, dialkylaminosulfonyl, —CONH₂, alkylaminocarbonyl, dialkylaminocarbonyl, 3, 4 or 5 membered monocyclic heterocyclyl, hydroxy, alkoxy, cyano, halo, haloalkyl or haloalkoxy; and
R⁶ and R⁷ are independently hydrogen, alkyl, hydroxy, alkoxy, halo, haloalkyl, haloalkoxy, carboxy, alkoxycarbonyl, cyano, —CONH₂, amino, or monosubstituted and disubstituted amino;
or a pharmaceutically acceptable salt thereof.

2. The compound or salt of claim 1 wherein: 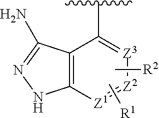
is

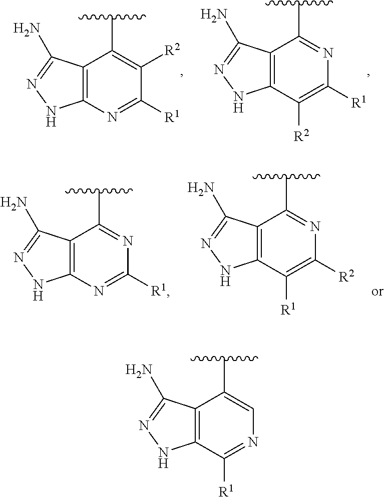

3. The compound or salt of claim 1 wherein: 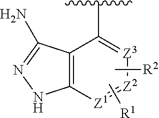
is

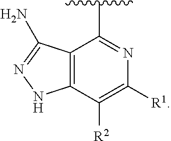

4. The compound or salt of claim 1 wherein: 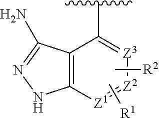
is

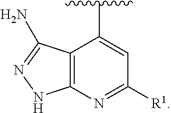

5. The compound or salt of any of the claims 1-4 wherein L is O, S, NH, or N(methyl), NHCO, CONH, or NHCONH.

6. The compound or salt of any of the claims 1-4 wherein L is O.

7. The compound or salt of any of the claims 1-4 wherein L is NHCONH.

8. The compound or salt of any of the claims 1-7 wherein R³ and R⁴ are independently hydrogen, alkyl, alkoxy, cyano, halo, haloalkyl or haloalkoxy.

9. The compound or salt of any of the claims 1-7 wherein R³ and R⁴ are independently hydrogen, methyl, fluoro, methoxy, chloro, trifluoromethyl, or trifluoromethoxy.

10. The compound or salt of any of the claims 1-7 wherein 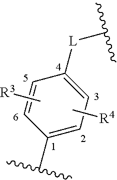
is a ring of formula: 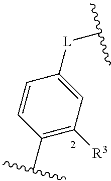
where R³ is hydrogen, methyl, ethyl, chloro, fluoro or trifluoromethyl.

11. The compound or salt of any of the claims 1-7 wherein 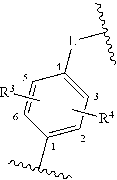
is a ring of formula: 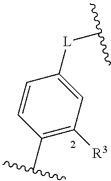
where R³ is hydrogen or fluoro.

12. The compound or salt of any of the claims 1-11 wherein:
R⁵ is hydrogen, alkyl, hydroxy, alkoxy, halo, haloalkyl, or haloalkoxy;
R¹ is —Z-(EWG′)-C(R^{b})═CHR^{c}; and
L is O.

13. The compound or salt of any of the claims 1-12 where Z is bond or alkylene, EWG′ is —NR′CO—, —NR′SO₂—, 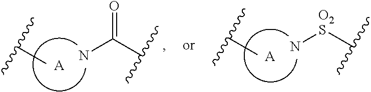
and R^{c} is alkyl, substituted alkyl, haloalkoxy, cycloalkyl, cycloalkyleneNR^{d}R^{e} or 3 to 6 membered saturated monocyclic heterocyclyl containing one or two heteroatoms selected from N, O, or S and optionally substituted with one or two substituents selected from hydroxy, alkyl or fluoro

14. The compound or salt of any of the claims 1-13 wherein R⁶ and R⁷ are independently hydrogen, alkyl, alkoxy, halo, haloalkyl, haloalkoxy, or cyano.

15. The compound or salt of any of the claims 1-13 wherein R⁶ and R⁷ are independently hydrogen, methyl, methoxy, fluoro, chloro, trifluoromethyl, trifluoromethoxy, or cyano.

16. The compound or salt of any of the claims 1-13 wherein R⁷ is hydrogen and 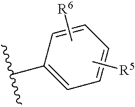
is a ring of formula:

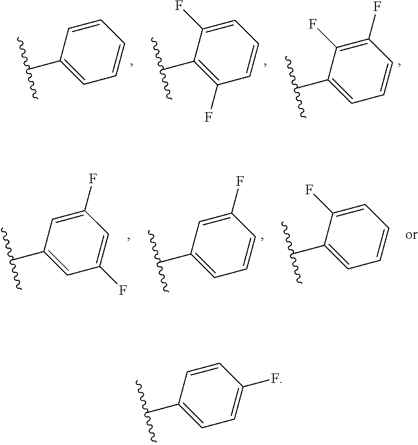

17. The compound or salt of any of the claims 1-13 wherein R⁷ is hydrogen and 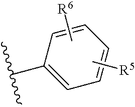
is a ring of formula:

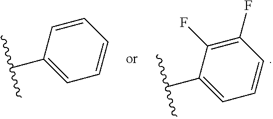 [SMILES: *c1ccccc1 ; *c1cccc(F)c1F]

18. The compound or salt of any of the claims 1-17 wherein:
Z is bond or alkylene; EWG′ is 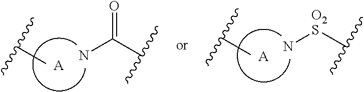
where ring A is heterocycloamino; R^{b} is cyano and R^{c} is isopropyl, tert-butyl, cyclopropyl, 1-methyl-1-methylaminoethyl, 1-methyl-1-dimethylaminoethyl, 1-methyl-1-aminoethyl, 1-methylaminocycloprop-1-ylene, 1-dimethylaminocycloprop-1-ylene, 1-ethoxy-1-methylethyl, —C(CH₃)₂-morpholine-4-yl, 2-pyrrolidinyl, 3- or 4-piperidinyl, 1-methylpiperidin-4-yl, 1-methylpiperidin-3-yl, or 4-tetrahydropyranyl.

19. The compound or salt of any of the claims 1-17 wherein:
—Z-EWG′- is: 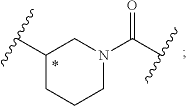 [SMILES: *C(=O)N1CCCC(*)C1]
R^{b} is cyano and R^{c} is isopropyl, tert-butyl, cyclopropyl, 1-methyl-1-methylaminoethyl, 1-methyl-1-dimethylaminoethyl, 1-methyl-1-aminoethyl, 1-methylaminocycloprop-1-ylene, 1-dimethylaminocycloprop-1-ylene, 1-ethoxy-1-methylethyl, —C(CH₃)₂morpholine-4-yl, 2-pyrrolidinyl, 3- or 4-piperidinyl, 1-methylpiperidin-4-yl, 1-methylpiperidin-3-yl, or 4-tetrahydropyranyl and the stereochemistry at *C is (R) or (S).

20. The compound or salt of any of the claims 1-17 wherein:
—Z-EWG′- is: 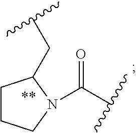 [SMILES: *CC1CCCN1C(*)=O]
R^{b} is cyano and R^{c} is isopropyl, tert-butyl, cyclopropyl, 1-methyl-1-methylaminoethyl, 1-methyl-1-dimethylaminoethyl, 1-methyl-1-aminoethyl, 1-methylaminocycloprop-1-ylene, 1-dimethylaminocycloprop-1-ylene, 1-ethoxy-1-methylethyl, —C(CH₃)₂morpholine-4-yl, 2-pyrrolidinyl, 3- or 4-piperidinyl, 1-methylpiperidin-4-yl, 1-methylpiperidin-3-yl, or 4-tetrahydropyranyl and the stereochemistry at **C is (R) or (S).

21. The compound or salt of any of the claims 1-20 wherein 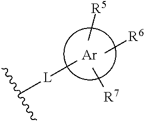
group is attached at the 4-position of the phenyl ring in

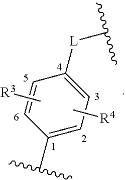

22. A pharmaceutical composition comprising a compound of any of the claims 1-21, or a pharmaceutically acceptable salt thereof; and a pharmaceutically acceptable excipient

23. A method of treating a disease treatable by inhibition of a kinase in a patient which method comprises administering to the patient in need thereof, a pharmaceutical composition comprising a compound of any of the claims 1-22 or a pharmaceutically acceptable salt thereof, and a pharmaceutically acceptable excipient.

24. The method of claim 23 wherein the kinase is BTK.

25. The method of any of the claims 23 or 24 wherein the disease is an inflammatory disease or cancer and the compound or salt of any of the claims 1-22 is administered optionally in combination with one or more anticancer or anti-inflammatory agent.
